(19) United States
(12) Patent Application Publication
(10) Pub. No.: US 2026/0144895 A1
(43) Pub. Date: May 28, 2026

(54) ADJUSTABLE GENE THERAPY

(71) Applicant: Remedium Bio, Inc., Needham, MA (US)

(72) Inventors: Alexei Goraltchouk, Cambridge, MA (US); Alexey Seregin, Westford, MA (US); Frank Luppino, Needham, MA (US)

(73) Assignee: Remedium Bio, Inc., Needham, MA (US)

(21) Appl. No.: 19/357,344

(22) Filed: Oct. 14, 2025

Related U.S. Application Data
(63) Continuation of application No. PCT/US2024/024375, filed on Apr. 12, 2024.
(60) Provisional application No. 63/459,417, filed on Apr. 14, 2023.

(51) Int. Cl. A61K 48/00; A61K 38/18; A61K 38/20; A61K 38/26; A61K 38/28

(52) U.S. Cl. CPC A61K 48/005; A61K 38/185; A61K 38/2006; A61K 38/26; A61K 38/28; A61K 48/0033

(57) ABSTRACT

The present invention applies, at least in part, to therapeutic agents, and more specifically to gene therapy for use in human or veterinary medicine.

### CROSS-REFERENCE TO RELATED APPLICATIONS

This application is a Continuation of International Patent Application Serial No. PCT/US2024/024375, filed Apr. 12, 2024, which claims the benefit under 35 U.S.C. § 119 (e) of U.S. Provisional Application No. 63/459,417, filed Apr. 14, 2023, both entitled “ADJUSTABLE GENE THERAPY,” the disclosure of each of which is hereby incorporated by reference in its entirety.

### REFERENCE TO AN ELECTRONIC SEQUENCE LISTING

The contents of the electronic sequence listing (R087270003US01-SEQ-JAV.xml; Size: 132,722 bytes; and Date of Creation: Oct. 8, 2025) are herein incorporated by reference in its entirety.

### FIELD

The present invention applies, at least in part, to therapeutic agents, and more specifically to gene therapy for use in human or veterinary medicine.

### BACKGROUND

Recent advances in gene therapy have demonstrated that this treatment modality is not only capable of curing, reducing pathology, or preventing progression of diseases, such as monogenic diseases, by replacing a defective or malfunctioning gene, but can also serve as a means of gene augmentation when natural expression or protein function is insufficient. This allows for the use of gene therapy in a number of applications previously considered sub-optimal for treatment by gene delivery. However, improvements are needed.

### SUMMARY

Aspects of the present disclosure relate to an adjustable therapeutic gene therapy system for the delivery to, for example, any one of the tissues or cells provided herein, such as to the subcutaneous space (e.g., to one or more cell types in the subcutaneous space), which system comprises: a) a genetic construct encoding at least one therapeutic gene, under the regulation of at least one promoter element and, optionally, one or more regulatory elements, b) a delivery carrier or vector encapsulating at least part of said genetic construct, optionally, which allows for the re-administration of a subsequent dose of the genetic construct (e.g., at an efficiency of at least 10% as compared to an initial dose of the genetic construct), and c) a means of increasing or decreasing the level of therapeutic transgene expression from the genetic construct.

In some embodiments, the means of c) is a means for permanently reducing the level of therapeutic transgene expression (e.g., by at least 2%).

In some embodiments, the means for reducing the level of therapeutic transgene expression is via application of an external stimulus.

In some embodiments, the genetic construct encodes at least one functional part of a human peptide or protein (e.g., a full-length human protein). In some embodiments, the genetic construct encodes at least one functional part of a human protein analog or antagonist. In some embodiments, the genetic construct encodes at least one functional part of a non-human peptide or protein (e.g., a full-length non-human protein). In some embodiments, the genetic construct encodes at least one functional part of a non-human protein analog or antagonist. In some embodiments, the genetic construct encodes insulin or an insulin analog. In some embodiments, the insulin or insulin analog is a modified, furin-cleavable insulin. In some embodiments, the genetic construct encodes a GLP-1, GLP-1 agonist, or GLP-1 agonist analog. In some embodiments, the genetic construct encodes a growth factor. In some embodiments, the genetic construct encodes a cytokine. In some embodiments, the genetic construct encodes an anti-inflammatory protein. In some embodiments, the genetic construct encodes a complement protein. In some embodiments, the genetic construct encodes a receptor agonist. In some embodiments, the genetic construct encodes a receptor antagonist. In some embodiments, the genetic construct encodes a fusion protein (e.g. comprised of one or more functional elements of different proteins). In some embodiments, the subcutaneous space is the hypodermal layer.

In some embodiments, the subcutaneous space is the layer between the superficial and deep fascia. In some embodiments, the subcutaneous space is the layer between the dermis and the superficial fascia. In some embodiments, the genetic construct is comprised of one or more circular single-stranded DNA constructs. In some embodiments, the genetic construct is comprised of one or more circular double-stranded DNA constructs. In some embodiments, the genetic construct is comprised of one or more linear single-stranded DNA constructs. In some embodiments, the genetic construct is comprised of one or more linear double-stranded DNA constructs. In some embodiments, the genetic construct is at least in part comprised of DNA or RNA. In some embodiments, the genetic construct is comprised of DNA and/or RNA. In some embodiments, the external stimulus is comprised of chemical DNA-, RNA-, or DNA- and RNA-derivatives.

In some embodiments, the promoter element comprises a constitutive promoter. In some embodiments, the promoter element comprises an inducible promoter. In some embodiments, the promoter element comprises a tissue specific promoter. In some embodiments, the promoter element comprises a promoter containing a sequence from one or more of the CAG, EF1a, UBC, CBh, MSCV, hPGK, SFFV, and SV40 promoters. In some embodiments, the promoter element comprises a tetOn inducible promoter construct. In some embodiments, the promoter element comprises at least a promoter sequence with one or more of an enhancer, a regulator, an operator, and/or a repressor. In some embodiments, the promoter element comprises an inducible promoter that can up- or down-regulate expression in response to external or internal stimuli, such as inflammation, heat, light, stress, administration of steroids, tetracycline, antibiotics, rapamycin, ganciclovir, acyclovir), or is inducible by up- or down-regulated molecules (e.g., ROS, NOS, or cytokine release). In some embodiments, the promoter element comprises a circadian rhythm or cycling promoter (e.g., that changes its level of activity by at least 5% with some periodicity ranging from hours to months).

In some embodiments, the regulatory element comprises a post translational regulatory element. In some embodiments, the regulatory element comprises at least a part of or all of the Woodchuck Hepatitis Virus Posttranscriptional Regulatory Element or WPRE. In some embodiments, the regulatory element comprises at least a part of or all of an optimized Woodchuck Hepatitis Virus Posttranscriptional Regulatory Element or WPRE. In some embodiments, the regulatory element comprises a cis-acting element that can increase accumulation of cytoplasmic mRNA. In some embodiments, the regulatory element comprises at least a part of or all of the HIV type 1 Rev-Rev-responsive element. In some embodiments, the regulatory element comprises at least a part of or all of the human hepatitis virus post-translational regulatory element. In some embodiments, the regulatory element comprises at least a part of or all of one or more viral post-translational regulatory elements that can improve expression of the gene of interest or any other element encoded by the genetic construct.

In some embodiments, the genetic construct comprises one or more functional genetic sequences. In some embodiments, the one or more functional genetic sequences promote translocation of the genetic construct (e.g., of at least 4% of the genetic construct) to the nucleus of target cells. In some embodiments, the one or more functional genetic sequences comprises a sequence encoding a sequence-specific DNA binding protein coupled to a nuclear localization signal peptide. In some embodiments, the one or more functional genetic sequences comprises a DNA nuclear target sequence recognized by one or more specific transcription factors. In some embodiments, the one or more functional genetic sequences comprises a DNA nuclear target sequence (DTS) active in the importin-mediated transport system. In some embodiments, the one or more functional genetic sequences comprises at least a part of or all of the SV40 DTS. In some embodiments, the one or more functional genetic sequences comprises at least a part of or all of the glucocorticoid response element (GRE) DTS. In some embodiments, the one or more functional genetic sequences comprises a Sox2 regulatory region 2 DTS sequence. In some embodiments, the one or more functional genetic sequences comprises an importin beta (1), importin 7, NF-kappaBeta, or a small guanosine triphosphatase Ran interacting sequence. In some embodiments, the one or more functional genetic sequences (e.g., reduces expression from the genetic construct) comprises an inducible suicide gene. In some embodiments, the one or more functional genetic sequences (e.g., reduces expression from the genetic construct) comprises an inducible expression system encoding an RNA molecule that decreases expression of at least a portion of the therapeutic genetic construct. In some embodiments, the one or more functional genetic sequences (e.g., reduces expression from the genetic construct) encodes a protein capable of targeted epigenetic silencing. In some embodiments, the one or more functional genetic sequences (e.g., reduces expression from the genetic construct) encodes an HSV-TK suicide gene or system. In some embodiments, the one or more functional genetic sequences (e.g., reduces expression from the genetic construct) encodes a RapaCas9 suicide gene or system. In some embodiments, the one or more functional genetic sequences (e.g., reduces expression from the genetic construct) encodes one or more of miRNA, siRNA, shRNA, dsRNA, ncRNA, lncRNA, piwi-interacting RNA, PAT, eRNA, and/or circRNA. In some embodiments, the one or more functional genetic sequences (e.g., reduces expression from the genetic construct) encodes at least a guide RNA and CRISPR-dCas system (e.g., coupled to a protein that induces epigenetic silencing). In some embodiments, the one or more functional genetic sequences (e.g., reduces expression from the genetic construct) encodes at least a guide RNA and CRISPR-dCas9-KRAB. In some embodiments, the one or more functional genetic sequences (e.g., reduces expression from the genetic construct) encodes at least a guide RNA and CRISPR-dCas12b-KRAB. In some embodiments, the one or more functional genetic sequences (e.g., reduces expression from the genetic construct) encodes at least a guide RNA and CRISPR-dCas8c-KRAB. In some embodiments, the one or more functional genetic sequences (e.g., reduces expression from the genetic construct) encodes at least a guide RNA and CRISPR-dCas8a-KRAB. In some embodiments, the one or more functional genetic sequences (e.g., reduces expression from the genetic construct) encodes at least a guide RNA and CRISPR-dCas8b-KRAB. In some embodiments, the one or more functional genetic sequences (e.g., reduces expression from the genetic construct) encodes at least a TALEN, meganuclease, endonuclease, restriction enzyme, Zinc-Finger protein, or other DNA- or RNA-binding protein.

In some embodiments, the regulatory element changes the expression from the genetic construct in response to temperature. In some embodiments, the regulatory element changes the expression from the genetic construct in response to heat. In some embodiments, the regulatory element changes the expression from the genetic construct in response to cold. In some embodiments, the regulatory element changes the expression from the genetic construct in response to ultrasound. In some embodiments, the regulatory element changes the expression from the genetic construct in response to electrical stimuli. In some embodiments, the regulatory element changes the expression from the genetic construct in response to chemical stimuli. In some embodiments, the regulatory element changes the expression from the genetic construct in response to changes in the physiological environment. In some embodiments, the regulatory element changes the expression from the genetic construct in response to paracrine, endocrine, or autocrine factors. In some embodiments, the regulatory element changes the expression from the genetic construct in response to the presence of inflammation. In some embodiments, the regulatory element comprises a proximal promoter. In some embodiments, the regulatory element comprises a distal promoter. In some embodiments, the regulatory element comprises an insulator. In some embodiments, the regulatory element forms secondary structures with the genetic construct or other genetic sequences.

In some embodiments, the delivery carrier or vector comprises lipid nanoparticles (e.g., functionalized lipid nanoparticles, solid lipid nanoparticles, lipid-polymer hybrid nanoparticles). In some embodiments, the delivery carrier or vector comprises liposomes (e.g., functionalized liposomes, stealth liposomes) or micelles. In some embodiments, the delivery carrier or vector comprises one or more cells (e.g., functional cells), cellular components, or cell membranes. In some embodiments, the delivery carrier or vector comprises cubosomes, transfectosomes, endosomes, exosomes, or vesicular systems. In some embodiments, the delivery carrier or vector comprises polymeric nanoparticles. In some embodiments, the delivery carrier or vector comprises non-immunogenic or hypoimmunogenic viral vectors. In some embodiments, the delivery carrier or vector comprises proteins or polypeptides. In some embodiments, the delivery carrier or vector is functionalized to reduce immunogenicity. In some embodiments, the delivery carrier or vector is functionalized to promote uptake (e.g., by one or more specific cell types). In some embodiments, the delivery carrier or vector is functionalized to promote endocytosis, pinocytosis, or enhance transport to the nucleus. In some embodiments, the delivery carrier or vector comprises an ionizable lipid. In some embodiments, the delivery carrier or vector comprises an ionizable element. In some embodiments, the delivery carrier or vector comprises an ionizable polymer. In some embodiments, the delivery carrier or vector comprises cholesterol. In some embodiments, the delivery carrier or vector comprises a cationic component. In some embodiments, the delivery carrier or vector comprises an amphiphilic polymer. In some embodiments, the delivery carrier or vector comprises polyethylene glycol. In some embodiments, the delivery carrier or vector comprises an antibody, nanobody, or antibody fragment. In some embodiments, the delivery carrier or vector comprises a peptide. In some embodiments, the delivery carrier or vector comprises a nanoparticle chemically or physically conjugated with peptides, proteins, or functional sequences derived from peptides or proteins. In some embodiments, the delivery carrier or vector comprises a nanoemulsion, nanostructured lipid, or comprised at least in part of an amphiphilic polymer or oligomer. In some embodiments, the delivery carrier or vector comprises a phospholipid, sphingolipid, polyelectrolytic polymer, or polyelectrolytic complex. In some embodiments, the delivery carrier or vector comprises metallic or ceramic nanoparticles. In some embodiments, the delivery carrier or vector elicits no humoral response or no cell mediated immune response. In some embodiments, the delivery carrier or vector elicits no memory immune response. In some embodiments, the delivery carrier or vector is minimally immunogenic (e.g., such that the memory immune response generated by the carrier or vector is incapable of neutralizing at least 10% of a subsequent administration of the carrier or vector via the same route of administration, such as in a test subject). In some embodiments, the delivery carrier or vector is minimally immunogenic (e.g., such that the memory immune response generated by the carrier or vector is incapable of neutralizing at least 50% of a subsequent administration of the carrier or vector via the same route of administration, such as in a test subject). In some embodiments, the delivery carrier or vector is minimally immunogenic (e.g., such that the memory immune response generated by the carrier or vector is incapable of neutralizing at least 75% of a subsequent administration of the carrier or vector via the same route of administration, such as in a test subject). In some embodiments, the delivery carrier or vector is minimally immunogenic (e.g., such that the memory immune response generated by the carrier or vector is incapable of neutralizing at least 90% of a subsequent administration of the carrier or vector via the same route of administration, such as in a test subject). In some embodiments, the delivery carrier or vector is minimally immunogenic (e.g., such that the memory immune response generated by the carrier or vector is incapable of neutralizing at least 99% of a subsequent administration of the carrier or vector via the same route of administration, such as in a test subject).

In some embodiments, the external stimulus elicits cooling of cutaneous and sub-cutaneous tissues (e.g., to cause adipocyte cell death). In some embodiments, the external stimulus elicits cooling of cutaneous and sub-cutaneous tissues to cause adipocyte senescence. In some embodiments, the external stimulus causes a change in temperature of the sub-cutaneous tissues sufficient to change expression from the genetic construct. In some embodiments, the external stimulus comprises high-frequency ultrasound. In some embodiments, the external stimulus comprises medium-frequency ultrasound. In some embodiments, the external stimulus comprises low-frequency ultrasound. In some embodiments, the external stimulus comprises high-frequency ultrasound mediated cavitation. In some embodiments, the external stimulus comprises medium-frequency ultrasound mediated cavitation. In some embodiments, the external stimulus comprises low-frequency ultrasound mediated cavitation. In some embodiments, the external stimulus comprises ultrasound at a frequency in the range of 1-10 KHz. In some embodiments, the external stimulus comprises ultrasound at a frequency in the range of 10-28 KHz. In some embodiments, the external stimulus comprises ultrasound at a frequency in the range of 28-40 KHz. In some embodiments, the external stimulus comprises ultrasound at a frequency in the range of 40-60 KHz. In some embodiments, the external stimulus comprises ultrasound at a frequency range of 1-320 KHz. In some embodiments, the external stimulus comprises sound waves (e.g., capable of suppressing expression in adipocytes, inducing sentence in adipocytes, or inducing adipocyte cell death, such as in at least a part of the sub-cutaneous space). In some embodiments, the external stimulus comprises electromagnetic waves (e.g., capable of suppressing expression in adipocytes, inducing sentence in adipocytes, or inducing adipocyte cell death, such as in at least a part of the sub-cutaneous space). In some embodiments, the external stimulus comprises electrical signals (e.g., capable of suppressing expression in adipocytes, inducing sentence in adipocytes, or inducing adipocyte cell death, such as in at least a part of the sub-cutaneous space). In some embodiments, the external stimulus comprises administration of a formulation containing Rapamycin or a rapalog or a derivative of Rapamycin. In some embodiments, the external stimulus comprises administration of a formulation containing Tetracycline or its derivative. In some embodiments, the external stimulus comprises administration of Rapamycin or a rapalog or a derivative of Rapamycin in combination with Tetracycline or a derivative of Tetracycline. In some embodiments, the external stimulus comprises administration of Gancyclovir or a derivative of Gancyclovir. In some embodiments, the external stimulus comprises administration of Doxycycline or a derivative of Doxycycline. In some embodiments, the external stimulus comprises administration of one or more pharmacologically active small molecules. In some embodiments, the external stimulus comprises administration of one or more peptides with or without conjugation. In some embodiments, the external stimulus comprises administration of one or more hormones or their analogs. In some embodiments, the external stimulus comprises administration of tamoxifen or a derivative of tamoxifen. In some embodiments, the external stimulus comprises administration of one or more agents that elicit an inflammatory response. In some embodiments, the external stimulus comprises administration of one or more steroids. In some embodiments, the external stimulus comprises administration of one or more antibodies with or without functionalization. In some embodiments, the external stimulus comprises administration of one or more receptor ligands. In some embodiments, the external stimulus comprises administration of one or more antibody fragments. In some embodiments, the external stimulus comprises administration of one or more proteins. In some embodiments, the external stimulus comprises administration of one or more fusion proteins. In some embodiments, the external stimulus comprises administration of one or more RNA molecules. In some embodiments, the external stimulus comprises administration of one or more lipids. In some embodiments, the external stimulus comprises administration of one or more metabolites. In some embodiments, the external stimulus comprises administration of an agents that elicits an anti-inflammatory or immunomodulatory response. In some embodiments, the external stimulus comprises administration of one or more neurotransmitters. In some embodiments, the external stimulus comprises administration of one or more proteins capable of directly or indirectly interacting with a promoter, RNA or DNA sequence. In some embodiments, the external stimulus comprises administration of one or more RNA or DNA molecules capable of directly or indirectly interacting with a promoter, RNA or DNA sequence.

Aspects of the present disclosure relate to a method of therapeutic transgene expression comprising, administering the adjustable therapeutic gene therapy system described herein at least once.

Aspects of the present disclosure relate to a method of therapeutic transgene expression comprising, administering a genetic construct encoding at least one therapeutic gene, under the regulation of at least one promoter element and, optionally, one or more regulatory elements, and, optionally, a delivery carrier or vector encapsulating at least part of said genetic construct, to any one of the tissues or cells provided herein, such as to the subcutaneous space (e.g., to one or more cell types in the subcutaneous space).

In some embodiments, the genetic construct is any one of the genetic constructs as described herein.

In some embodiments, the one or more regulatory elements is any one of the regulatory elements as described herein.

In some embodiments, the delivery carrier or vector is any one of the carriers or vectors as described herein.

In some embodiments, the method further comprises killing, reducing, removing or causing senescence of cells in the subcutaneous space to which the genetic construct has been administered. In some embodiments, the method further comprises decreasing transgene expression from the genetic construct. In some embodiments, the method further comprises increasing transgene expression from the genetic construct.

In some embodiments, an external stimulus is applied to cells in the subcutaneous space to which the genetic construct has been administered. In some embodiments, the external stimulus is any one of the external stimuli as described herein. In some embodiments, the external stimulus is heat, cold, electromagnetic radiation, ultrasound, sound waves, pressure, electrical stimulation, or other chemical or physical means to increase or decrease expression of the therapeutic transgene or an element that can increase or decrease expression of the therapeutic transgene.

In some embodiments, the adjustable gene therapy system and/or genetic construct comprises a means for up- or down-regulation of transgene expression, optionally, wherein the means is any one of the means for such purpose provided herein.

In some embodiments, the method further comprises administering a second dose of the i) adjustable gene therapy system or ii) the genetic construct encoding at least one therapeutic gene, under the regulation of at least one promoter element and, optionally, one or more regulatory elements, and, optionally, a delivery carrier or vector encapsulating at least part of said genetic construct, to the subcutaneous space (e.g., to one or more cell types in the subcutaneous space).

In some embodiments, transgene expression is permanently reduced (e.g., relative to the baseline level of expression of the administered transgene).

In some embodiments, the administration is for treating disease in a subject (e.g., human subject). In some embodiments, the administration is for treating disease in a non-human subject. In some embodiments, the administration is for augmenting or enhancing human or animal health, physical condition, mental state, or physical or mental abilities. In some embodiments, the administration is for increasing human or animal longevity and/or increasing human or animal healthspan.

In some embodiments, the target cell is an adipocyte. In some embodiments, the target cell is a pre-adipocyte. In some embodiments, the target cell is a progenitor capable of differentiating into an adipocyte. In some embodiments, the target cell is a stem cell capable of differentiating into an adipocyte. In some embodiments, the target cell is a resident cell of the subcutaneous tissue. In some embodiments, the target cell is a transient cell or stem cell, but can become a resident cell of the subcutaneous tissue.

In some embodiments, the adjustable therapeutic system or method is for the treatment of monogenic disorders. In some embodiments, the adjustable therapeutic system or method is for the treatment of enzyme insufficiency disorders. In some embodiments, the adjustable therapeutic system or method is for protein replacement therapy. In some embodiments, the adjustable therapeutic system or method is for the treatment of metabolic disorders. In some embodiments, the adjustable therapeutic system or method is for the treatment of autoimmune disorders. In some embodiments, the adjustable therapeutic system or method is for the treatment of oncology. In some embodiments, the adjustable therapeutic system or method is for the treatment of neurologic disorders. In some embodiments, the adjustable therapeutic system or method is for the treatment of cardiovascular pathologies. In some embodiments, the adjustable therapeutic system or method is for the treatment of musculoskeletal disorders. In some embodiments, the adjustable therapeutic system or method is for the treatment of hematologic disorders. In some embodiments, the adjustable therapeutic system or method is for the treatment of dermatologic disorders. In some embodiments, the adjustable therapeutic system or method is for the treatment of immune system disorders. In some embodiments, the adjustable therapeutic system or method is for the treatment of pulmonary system disorders. In some embodiments, the adjustable therapeutic system or method is for the treatment of kidney or bladder disorders. In some embodiments, the adjustable therapeutic system or method is for the delivery of therapeutic antibodies via expression from the genetic construct. In some embodiments, the adjustable therapeutic system or method is for the delivery of therapeutic proteins via expression from the genetic construct. In some embodiments, the adjustable therapeutic system or method is for the delivery of fusion proteins via expression from the genetic construct. In some embodiments, the adjustable therapeutic system or method is for the delivery of peptides via expression from the genetic construct. In some embodiments, the adjustable therapeutic system or method is for the delivery of immunogens via expression from the genetic construct. In some embodiments, the adjustable therapeutic system or method is for the delivery of vaccines via expression from the genetic construct.

In some embodiments, the adjustable therapeutic system or method further comprises, or comprises the administration thereof, one or more markers for subsequent localization, visualization, analysis, enablement of up-titration, enablement of down-titration, selection of subsequent dosing, or general visualization, or the means for the foregoing. In some embodiments, the adjustable therapeutic system or method further comprises, or comprises the administration thereof, superficial or internal markers for subsequent localization, visualization, analysis, enablement of up-titration, enablement of down-titration, selection of subsequent dosing, or general visualization, or the means for the foregoing, at the delivery site.

In some embodiments, the one or more markers comprise any one of the markers as described herein (e.g., one or more of a metal, ceramic, polymer, or composite, or genetic code encoding a reporter protein, optionally, that can be visualized directly or following application of a stimulus to allow or enhance visualization).

In some embodiments, the stimulus is any one of the stimuli as described herein (e.g., comprises one or more of energy, cold, heat, administration of one or more chemical, therapeutic, molecule or atom to change the physical, chemical, or physiological state of the system to enable marker reporting, visualization, readout, or interaction thereby providing information about the treatment).

In some embodiments, the administration occurs more than once, such as multiple times, during a treatment.

### BRIEF DESCRIPTION OF THE DRAWINGS

FIGS. 1A-1B are plasmid maps for npRB2.6v3 (CBh-FC-hINS-CMV-rapaCasp9-K19) (FIG. 1A) and npRB2.6v7 (CBh-FC-FLuc-CMV-rapaCasp9-K19) (FIG. 1B).

FIG. 2 shows results of a cellular apoptosis assay, quantified by FLuc expression at 76 hpi.

FIG. 3 shows RapaCasp9-induced apoptosis in HdAD at 24 hpi. Results are shown for 0 mM, 0.05 mM and 0.1 mM Tet.

FIG. 4 is a plasmid map for gcCBh-fLuc-SV40IpA.

FIGS. 5A-5C show results of animal monitoring post subcutaneous injection of an initial construct dose. FIGS. 5A-5B show IVIS confirmed the durability of the initial signal (transgene expression) through Day 22. FIGS. 5A and 5C demonstrate the ability to up-titrate the initial dose in a predictable dose-dependent manner.

FIG. 6 shows the total flux from a region of interest as a function of Time (days).

FIG. 7 shows the total flux from ROI up titrations after one week (left) and one month (right.)

FIG. 8 shows the average flux in albino mice pre- and post-down-titration.

FIG. 9 is a plasmid map for npRB2.10v1 (EFS-NLuc+EFS-Cre-ERT2).

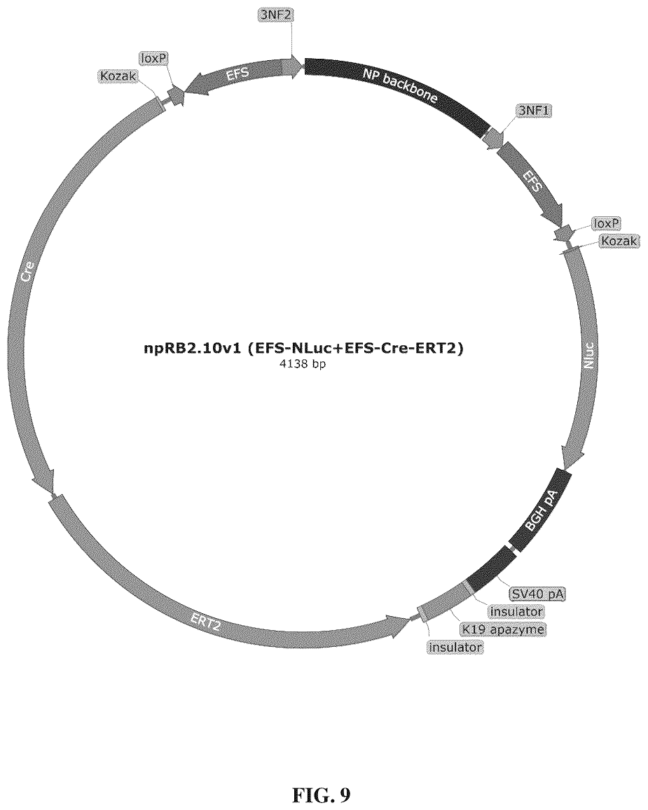

FIG. 10 is a plasmid map for npRB2.6v8 (CBh-NLuc).

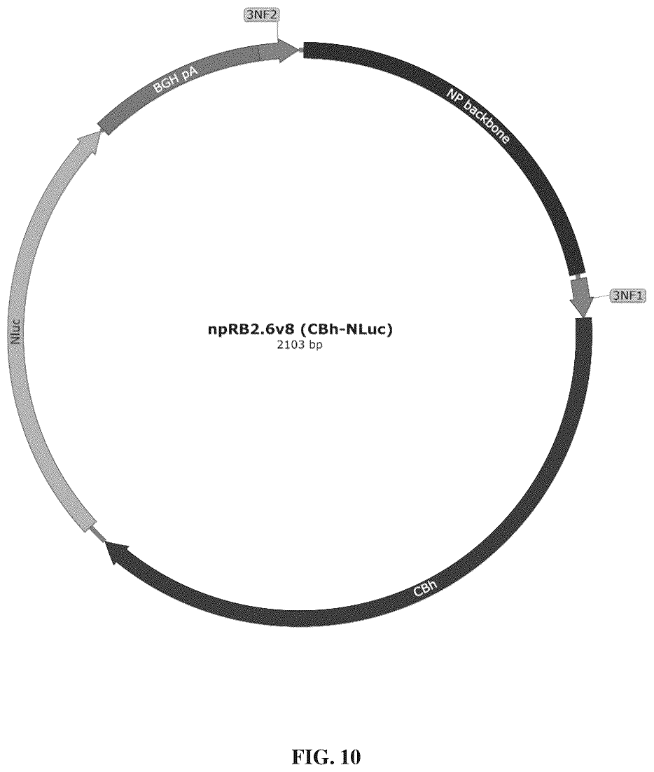

### DETAILED DESCRIPTION

In its current form, gene therapy is a single dose treatment, delivering an initial dose without the ability to up- or down-titrate that dose following initial administration. Additionally, gene therapy treatments delivered by viral vectors cannot be re-dosed as the viral vectors elicit a memory immune response that neutralizes the subsequent dose, often in a manner that is variable patient to patient and organism to organism, and can depend on a number of factors such as the initial route of administration, delivered dose, immune state of the patient, pre-existing immunity to the vector, and immune system state at the time of potential re-dosing. Moreover, several in vivo studies have demonstrated variable responses to re-dosing of viral gene therapies within the same organism and using the same vector, with some subsequent doses demonstrating similar or equivalent level of expression to the originally delivered dose, and other delivered doses demonstrating no expression from the therapeutic construct. This variability is hypothesized to be caused by the aforementioned immune and vector-related factors, which create a lack of predictability for re-treatment, up-titration, or dose augmentation. Targeted particles have been used in drug delivery and virus-like particles have been attempted as a means of narrowing the tropism of gene therapy treatments, the targeting moieties in such particles also prevent the re-dosing of gene therapies and therefore up-titration following initial administration. In addition, while attempting to target tissues, the majority of gene therapies are administered intravenously or directly to the organ requiring the gene replacement. Intravenously administered gene therapies are predominantly taken up by the liver, regardless of whether viral or non-viral vectors are used and regardless of whether targeting or untargeted particles are applied. Gene therapies delivered to the target organ, can be effective at transducing target tissues, but are also at times associated with increased complications due to the immunogenic reaction within that organ, difficulty accessing the target tissues (such as the brain or retina), and overall complexity of the procedure. Conversely, the majority of protein replacement therapies or protein-based therapeutic interventions are administered subcutaneously, which is a route of administration that provides simple access, excellent bioavailability, and is generally to a tissue that can withstand insult without a major impact to the overall functionality of the organism.

Moreover, while gene therapies using non-immunogenic carriers have been attempted, these treatments lack the ability to down-titrate the initially administered dose in a controllable and predictable manner. An inability to titrate gene therapy treatments post initial delivery prevents the use of gene therapy in a number of applications that require up- and down-titration of the therapeutic following an initial assessment of its safety and efficacy in a given patient. At the same time, many therapeutics require up-titration and down-titration for effective function, as an example GLP-1 receptor agonists (or GLP-1 analogs) and basal insulin are administered initially at low doses to assess the patient specific tolerability of the treatment, and following initial assessment, which can last weeks to months, the treatment dose is reviewed for adjustment to optimize its safety, tolerability, or efficacy. Other treatments require a down-titration of the initial dose due to attainment of efficacy and the need to reduce safety related risks. As an example, anti-VEGF therapy used for Wet Age-Related Macular Degeneration is initially administered at high frequencies, or a high average in situ concentration, which is eventually reduced (in dose or in frequency) to minimize potential treatment related side-effects. As such, in order to expand the applicability of gene therapy to treatment that require or could benefit from dose adjustment following initial administration, a dose-adjustable or titratable gene therapy has been developed, allowing for at least one, but in some embodiments multiple optional dose up- and/or down-titrations following initial delivery. Finally, the route of administration of said gene therapy, is preferably, in some embodiments, optimal to allow for effective up- and down-titration, safety profile of the treatment, and/or appropriate pharmacokinetics for a broad range of therapeutic applications. Accordingly, provided herein is a means of enabling and using an adjustable gene therapy treatment, which can be delivered at an initial dose and subsequently up- or down-titrated post initial administration, and related compositions and methods.

The therapeutic gene therapy formulations provided herein can be used for the treatment of diseases or management of disease symptoms. More specifically, the therapeutic formulations disclosed herewithin can be used for human or veterinary medicine and comprises at least one genetic construct or gene construct that encodes at least one therapeutic gene or genetic sequence capable of a beneficial effect in human or veterinary medicine. Provided herein are means of delivering a number of functional elements in cis or in trans with the therapeutic gene or genetic construct that enables or optimizes up- and/or down-titration of said therapeutic formulation or specifically the therapeutic gene or genetic construct following initial administration. In embodiments, element(s) for an adjustable dose gene therapy that can be up- or down-titrated at least once following initial administration is/are provided. The formulations provided herein can be used to optimize safety, efficacy, and/or tolerability, of the originally delivered dose. In embodiments, provided are means of delivering the gene therapy treatment to the sub-cutaneous space, which generally is sufficiently vascularized and can provide optimal pharmacokinetics for subcutaneously delivered biologics as well as a means of down-titration such as by application of an agent or means to reduce the level of expression of the gene therapy in said target cells, or optionally reduce the viability of the target cells, or optionally induce senescence in or of the target cells. Such agent or means may be one or more of ultrasound, focused ultrasound, high frequency ultrasound, intermediate or medium frequency ultrasound, low frequency ultrasound, ultrasound mediated cavitation, ultracavitation, radiofrequency, cold, temperature flux, cooling effect, reduction in temperature, electromagnetic radiation, and focused energy.

In general, provided herein is a therapeutic gene therapy formulation for the treatment of human or veterinary diseases or pathologies, which can also allow for up- and/or down-titration of the delivered dose post initial administration. The formulations may comprise at least a therapeutic gene construct of interest encoding at least one peptide, protein, or non-coding RNA and a promoter sequence that regulates expression of the therapeutic gene of interest. Any one of the formulations may be delivered in cis with a post translational regulatory element. Any one of the formulations may be delivered in cis or in trans with a genetic sequence that can promote translocation of at least a fraction of the therapeutic gene construct to the nucleus of target cells. Any one of the formulations may comprise at least one genetic sequence that can reduce expression from one or more elements of the gene construct by acting on the cell containing the gene construct, the gene construct itself, or one or more elements within the cell containing the gene construct. Any one of the genetic constructs may be at least partially encapsulated by a carrier or vector that does not elicit a memory immune response and/or is capable of neutralizing at least a fraction of subsequently re-administered therapeutic dose of the same or substantially similar composition via generally the same route of administration. For the therapeutic gene therapy systems described herein that do not contain regulatory sequences that enable down-titration of the therapeutic gene expression through silencing, gene construct elimination, or induction of apoptosis or general reduction of gene expression in the cell containing the construct, reduction in therapeutic transgene expression can be achieved via the application of an external stimulus that can eliminate the cell carrying the therapeutic construct, reduce the cells viability, general gene expression, general transcription, translation, or secretion of proteins, or induce apoptosis or senescence in the cell carrying the construct. Cells can be removed, such as by surgical means or other means, such as liposuction, in some embodiments.

Promotion of translocation to the nucleus may include translocation to the nucleus of a sufficient amount of the therapeutic construct to enable efficacy of the construct without compromising the safety or tolerability of the treatment and is ideally at least 4% of the construct and most ideally at least 20% of the construct on a molar basis. Similarly, in some embodiments, the development of an adaptive immune response includes the production of neutralizing antibodies or reactive T cells capable of eliminating or neutralizing the subsequent dose in a manner that limits its efficacy, with ideally less than 50% of the dose neutralized, most ideally less than 10% of the dose neutralized, and preferably no appreciable construct or carrier specific neutralization.

In some embodiments, the down-titration is complete, while in other embodiments the down-titration is incremental and can comprise as little as 2% of the overall expression to 15%, 90% or any amount between 2% and 100%. The incremental down-titration may be performed once, or in a series of stimulations to reduce the expression by multiples of the initial down-titration or as required to obtain optimal transgene expression.

Routes of administration for delivery of said formulations can be generally systemic or local and can be comprised of one or more of any clinically established routes of administration of a therapeutic agent. More specifically, the routes of administration may be local to one or more local tissues or organs, or systemic comprising enteral or parenteral routes of administration. In some embodiments, the routes of administration may include one or more of intravenous, sub-cutaneous, sub-dermal, intraarticular, intraventricular, intravenous, intramuscular, sub-arachnoid, vaginal, rectal, inhaled, intravitreal, oral, buccal, sublingual, ocular, transdermal, pulmonary, or intraperitoneal. In embodiments, one of the preferred methods of administration of the gene therapy is subcutaneous, or to adipose tissues of the subcutaneous space. This can allow for several rounds of up-titration if required, administration of a number of gene therapy constructs, and can be the safest means of down-titrating the gene therapy, while still affording the treatment optimal pharmacokinetics.

Provided herein are therapeutic formulations that contain genetically encoded therapeutic genes with or without functional elements or genetic elements involved in regulation. More specifically, the therapeutic formulations can encode at least one functional part of a human peptide or protein, at least one functional part of a human peptide or protein analog or antagonist, at least one functional part of a non-human peptide or protein, or at least one functional part of a non-human peptide or protein analog or antagonist. In some embodiments, the therapeutic formulations can include at least a part of a gene, or more specifically at least the coding segment of a gene. In yet more embodiments, the therapeutic gene of interest can encode a modified furin-cleavable insulin, insulin or insulin analog, GLP-1 peptide, GLP-1 agonist, or GLP-1 agonist analog. In yet other embodiments the therapeutic formulation can encode for at least a part of at least one growth factor, cytokine, anti-inflammatory protein, complement protein or generally a protein of the immune system or immune system regulator, as well as at least a part of a receptor agonist or antagonist, a secreted protein or a portion thereof, or a receptor or functional element thereof. In other embodiments the therapeutic formulations can encode at least a part of a fusion protein comprised of one or more functional elements of at least two different proteins or peptides. Alternatively, the therapeutic formulation can encode for non-protein coding elements of human or animal genomes that include generally structural or regulatory elements such as transfer RNA (tRNA), ribosomal RNA (rRNA), long non-coding RNA (lncRNA), micro RNA (miRNA), silencing RNA (siRNA), inducing RNA (iRNA), endogenous silencing-inducing RNA, piwi-interacting RNA (piRNA), or other types of RNA or DNA capable of performing intra- or extra-cellular function that may or may not involve coding for proteins.

The therapeutic gene constructs may contain at least one promoter driving or regulating the therapeutic gene or sequence of interest and optionally one or more additional promoters that can drive or regulate expression of the dose adjustment element, nuclear localization signal, or auxiliary elements that are necessary for the functionality of the therapeutic gene therapy or provide supplementary functionality.

In some embodiments the promoter sequence intended to drive expression of the therapeutic sequence can be one or more of a mammalian promoter, a constitutive promoter, an inducible promoter, or a tissue specific promoter. In other embodiments the promoter sequence can be derived at least in part from one or more of a viral promoter, bacterial promoter, an archaeal promoter, a promoter or regulatory sequence of a human or animal genome and may optionally contain introns, untranslated regions, enhancers, or other regulatory elements required for gene expression or regulation. In some embodiments the promoter sequence may contain one or more of at least elements of a chicken beta-Actin promoter and rabbit beta-Globin splice acceptor site (CAG) promoter, EF1alpha (EF1a) promoter, promoter of the polyubiquitin C gene (UBC) promoter, the CBh promoter comprised of an altered version of the CAG (Promoter consisting of cytomegalovirus (CMV) early enhancer element, the promoter region, the first exon, and the first intron of chicken beta-Actin gene, and the splice acceptor of the rabbit beta-Globin gene), murine stem cell virus (MSCV) promoter, phosphoglycerate kinase PGK promoter, spleen focus forming virus (SFFV) promoter, or simian virus 40 (SV40) promoter. The promoter sequence may be a tetracycline-On (tetOn) inducible promoter construct or a tetracycline-Off (tetOff) inducible promoter construct. Regulation may be additionally afforded by one or more enhancer, regulator, operator, or repressor sequences that may be delivered in cis or in trans with the construct. In some embodiments the promoter sequence may be comprised at least in part from an inducible promoter that can up- or down-regulate expression in response to external or internal stimuli such as inflammation, heat, light, stress, administration of steroids, tetracycline, antibiotics, rapamycin, ganciclovir, acyclovir, or is generally inducible by up- or down-regulated reactive oxygen species (ROS), reactive nitrogen species (NOS), or cytokine release. Moreover, in yet other embodiments the promoter sequence may vary its expression levels in response to internal or external stimulation by varying with natural organismal cycles. In some embodiments the promoter sequence may be comprised at least in part of a circadian rhythm or cycling promoter that changes its level of activity by at least 5%, or preferably by at least 50% with some periodicity ranging from hours to months, and preferably from days to weeks.

In other embodiments the promoter sequence intended to drive expression of auxiliary elements such as the dose adjustment sequence or peptides that promote nuclear localization, transport, stability, or regulate expression can be one or more of a mammalian promoter, a constitutive promoter, an inducible promoter, or a tissue specific promoter. In other embodiments the promoter sequence can be derived at least in part from one or more of a viral promoter, bacterial promoter, a promoter or regulatory sequence of a human or animal genome and may optionally contain introns, untranslated regions, enhancers, or other regulatory elements required for expression or regulation. In some embodiments the promoter sequence may contain one or more of at least elements of a CAG promoter, EF1alpha (EF1a) promoter, UBC promoter, CBh promoter, MSCV promoter, hPGK promoter, SFFV promoter, or SV40 promoter. The promoter sequence may be a tetOn inducible promoter construct or a tetOff inducible promoter construct. Regulation may be additionally afforded by one or more enhancer, regulator, operator, or repressor sequences that may be delivered in cis or in trans with the construct. In some embodiments the promoter sequence may be comprised at least in part from an inducible promoter that can up- or down-regulate expression in response to external or internal stimuli such as inflammation, heat, light, stress, administration of steroids, tetracycline, antibiotics, rapamycin, ganciclovir, acyclovir, or is generally inducible by up- or down-regulated ROS, NOS, or cytokine release. Moreover, in yet other embodiments the promoter sequence may vary its expression levels in response to internal or external stimulation by varying with natural organismal cycles. In some embodiments the promoter sequence may be comprised at least in part of a circadian rhythm or cycling promoter that changes its level of activity by at least 5%, or preferably by at least 50% with some periodicity ranging from hours to months, and preferably from days to weeks.

In embodiments, to allow for additional functionality and/or optimal expression of the therapeutic gene as well as any associated regulatory elements or sequences, the construct may contain one or more post translational regulatory elements that may include elements that regulate expression, conditionally regulate expression, stabilize the transcribed RNA, or form secondary or tertiary structures with one or more elements of the therapeutic construct, genomic DNA, or elements of other constructs delivered in cis or in trans. Some embodiments may contain one or more polyA signals or signals encoding polyA or regulating the length of the polyA tail, promote or enhance capping, promote or enhance ribozyme assembly or generally assembly or transcriptional or translational machinery (once said regulatory elements are transcribed). In some embodiments, the post translational regulatory elements included in the therapeutic construct may include at least a part of the Woodchuck Hepatitis Virus Posttranscriptional Regulatory Element or WPRE, at least a part of an optimized Woodchuck Hepatitis Virus Posttranscriptional Regulatory Element, a genetic sequence capable of increasing accumulation or stability of nuclear or cytoplasmic mRNA, at least a part of the HIV type 1 Rev-Rev-responsive element, at least a part of the human hepatitis virus post-translational regulatory element, at least a part of one or more viral post-translational regulatory elements capable of improving expression of the gene of interest or any other construct-encoded elements. In some embodiments, one or more post transcriptional regulatory elements can be included in series, in tandem, or delivered in trans with the construct. In yet other embodiments the regulatory elements may be upstream or downstream of the therapeutic gene or RNA-encoding sequence and may optionally regulate transcription, translation, or expression of auxiliary elements of the therapeutic construct.

In embodiments, to allow for additional or optional expression, construct localization, durability of the construct within the cell, and/or prevent an innate or adaptive immune response, the genetic sequence, elements of its carrier, or general formulation may contain one or more elements that promote translocation of said sequence to the nucleus or other cellular compartments or organelles. Said elements may be comprised at least in part of DNA, RNA, proteins, peptides, lipids, polymers, cholesterol, or other molecules capable of inducing the aforementioned effects of expression optimization, construct localization, stability, durability, and immune response optimization. In some embodiments the therapeutic construct contains a genetic sequence delivered in cis or in trans that promotes translocation of at least a fraction of the therapeutic construct, ideally at least 4% on a molar basis and ideally at least 20% of the gene construct on a molar basis to the nucleus of target cells. In some embodiments the genetic sequence is at least in part comprised of a protein coding sequence with a dedicated promoter, which encodes a protein to transport the therapeutic genetic construct to the nucleus. In other embodiments the genetic sequence is comprised at least in part of a sequence encoding a sequence-specific DNA binding protein coupled to a nuclear localization signal peptide, or at least in part of a DNA nuclear target sequence recognized by one or more specific transcription factors, or at least in part of a DNA nuclear target sequence (DTS) active in the importin-mediated transport system. In yet other embodiments the sequence promoting translocation of the therapeutic construct to the nuclease is comprised at least in part of the SV40 DTS, 3NF DTS, Sox2 regulatory region 2 DTS sequence, or an importin beta (1), importin 7, NF-kappaBeta, or the small guanosine triphosphatase Ran interacting sequence. In yet other embodiments the genetic sequence capable of inducing translocation of the gene construct or other elements delivered in trans to the nucleus encodes DNA capable of binding to intracellular transport proteins, or RNA capable of serving as an intermediary between the intracellular transport proteins and the therapeutic construct or elements delivered in trans.

In embodiments, to facilitate down-titration following initial administration of the gene therapy the therapeutic construct may contain one or more elements capable of reducing the expression level of the therapeutic gene of interest, protein, or RNA. The therapeutic construct may contain a genetic sequence capable of reducing expression from the therapeutic transgene upon activation by an external stimulus by modifying the levels of expression, altering the tertiary structure of the therapeutic construct transiently or permanently, eliminating the therapeutic construct from the cell, eliminating the cell carrying the therapeutic construct, inducing cellular senescence, generally reducing transcription within the cell containing the therapeutic construct, inducing apoptosis of the cell containing the therapeutic construct, or inducing epigenetic changes to the therapeutic construct, elements of the therapeutic construct or genomic DNA to alter transcription, translation, or generally expression of the therapeutic gene or RNA-encoding element contained within the therapeutic construct. In some embodiments the sequence capable of reducing expression from the therapeutic construct is a suicide gene, or an inducible suicide gene, or a suicide gene under an inducible promoter. In other embodiments the genetic sequence capable or reducing expression from the therapeutic construct is an inducible system encoding an RNA molecule capable of decreasing expression of at least a portion of the therapeutic genetic construct or a protein coding gene under the regulation of an inducible promoter, encoding a protein capable of targeted epigenetic silencing of at least a portion of the therapeutic genetic construct. In some embodiments the genetic sequence capable of reducing expression of the therapeutic construct is delivered in cis or in trans with the therapeutic construct and encodes one or more of elements of an Herpes Simplex Virus Thymidine Kinase or HSV-TK suicide gene system, rapamycin caspase 9 or RapaCas9 suicide gene system, or other suicide gene system capable of inducing programmed cell death or apoptosis of the cell containing the said construct. In yet other embodiments the genetic sequence capable of reducing expression of the therapeutic construct encodes one or more of miRNA, siRNA, shRNA, dsRNA, ncRNA, lncRNA, piwi-interacting RNA, PAT, eRNA, circRNA. In yet other embodiments the sequence encodes for a DNA binding protein with targeting capabilities that is capable through its domain or a coupled domain of inducing epigenetic changes in a sequence specific manner to reduce expression from the therapeutic construct. In other embodiments said sequence encodes at least a guide RNA and CRISPR-dCas system coupled to a protein capable of inducing epigenetic silencing, or at least a guide RNA and CRISPR-dCas9-KRAB, or at least a guide RNA and CRISPR-dCas12b-KRAB (KRAB=Krüppel-associated box protein functional fragment of the protein analog or homolog), or at least a guide RNA and CRISPR-dCas8c-KRAB, or at least a guide RNA and CRISPR-dCas8a-KRAB, or at least a guide RNA and CRISPR-dCas8b-KRAB. In other embodiments the genetic sequence capable of reducing expression of the therapeutic construct to enable dose down-titration encodes at least a Transcription activator-like effector nucleases or TALEN, meganuclease, endonuclease, restriction enzyme, Zinc-Finger protein, or other DNA- or RNA-binding protein.

In other embodiments, the means of down-titrating the therapeutic construct are contained within the genome, nucleus, mitochondria, or cytoplasm of the cell to which the therapeutic construct is delivered, and yet in another embodiment the means of down-titration are applied via an external stimulus such as ultrasound, heat, cold, thermal flux, electromagnetic radiation, non-electromagnetic radiation, impulse, pressure, or suction to the cells directly or indirectly, or to the tissues containing the cells or neighboring the tissues that contain at least some of the cells that carry the therapeutic construct. Said stimuli can induce cell death, necrosis, apoptosis, inflammation, reduction in the level of transcription or translation within the cell, senescence within the cell, or combinations thereof.

The therapeutic constructs described herein may be delivered without a carrier, encased in a carrier, partially encapsulated in a carrier, or interacting with a carrier in some physical or chemical way. Multiple carriers or carrier systems may be used to deliver the therapeutic construct and may include one or more of a liposome, a lipid nanoparticle, a polymeric nanoparticle, an exosome, microsome, nanosome, or exosome-like structure, an ionizable lipid containing particle, a nanoparticle containing lipids and polymers, a nanoparticle containing targeting ligands, a nanoparticle chemically or physically conjugated with peptides, proteins, or functional sequences derived from peptides or proteins, a lipid nanoemulsion, a solid lipid nanoparticle, a nanostructured lipid carrier. In some embodiments the carrier may contain one or more of amphiphilic oligomers, phospholipids, sphingolipids, ionizable lipids, or cholesterol. In yet other embodiments the carrier can be comprised at least in part from a polyelectrolytic complex, a cationic liposome, a stealth liposome, a cubosome, a lipoplex, a nanocell, or a lipid bilayer sheet. The carrier system or an element of the carrier can be bound with one or more targeting ligands, structures or molecules that enhance stability of the carrier, binding ligands, structures or molecules that promoter cellular uptake, increase the half-life of the carrier or therapeutic construct, promote internalization, facilitate endosomal escape, control, regulate, or promote intracellular trafficking, enhance transport to the nucleus or other cellular organelles.

Some specific examples of formulations and reagent concentration ranges that can be used to deliver the genetic cargo are listed below:

TABLE 1

| | | | N/P Ratio |
|---|---|---|---|
| Polymer ID | Formal Name | CAS # | Range |
| Chitosan* | beta-(1,4)-2-Amino-2- | 9012-76-4 | 1-15 |
| | deoxy-D-glucose | | |

*Chitosan degree of deacetylation can range from approximately 0.5 to approximately 99.9%, or a mixture of multiple degrees of acetylation. Molecular weight of chitosan can range from 5,000 Da to 2.5M Da.

TABLE 2

| | | | N/P Ratio |
|---|---|---|---|
| Polymer ID | Formal Name | CAS # | Range |
| Poly epsilon | Poly[imino[(2S)-2-amino- | 28211-04-3 | 10-70 |
| L-lysine* | 1-oxo-1,6-hexanediyl | | |

*Molecular weight of poly epsilon L-lysine can range from 1,500 Da to 250,000 Da.

TABLE 3

| | | | Molar | Range, |
|---|---|---|---|---|
| Lipid ID | Formal Name | CAS # | ratio | mol % |
| Coatsome | | | 48.5 | 15-65 |
| SS-OP | | | | |
| DOPC | 1,2-dioleoyl-sn-glycero-3- | 4235- | 7.5 | 0-35 |
| | phosphatidylcholine | 95-4 | | |
| Cholesterol | (3β)-cholest-5-en-3-ol | 57-88-5 | 42.5 | 0-55 |
| DMG- | α-[(2R)-2,3-bis[(1- | 160743- | 1.5 | 0-20 |
| PEG(2,000) | oxotetradecyl)oxy]propyl]- | 62-4 | | |
| | ω-methoxy-poly(oxy-1,2- | | | |
| | ethanediyl) | | | |

*Coatsome may be substitute for alternative similarly structure polymers in a comparable range of molar ratios.

TABLE 4

| | | | Molar | Range, |
|---|---|---|---|---|
| Lipid ID | Formal Name | CAS # | ratio, % | mol % |
| SM-102* | 8-[(2- | 2089251- | 50 | 15-65 |
| | hydroxyethyl)[6-oxo-6- | 47-6 | | |
| | (undecyloxy)hexyl]amino]- | | | |
| | octanoic acid, | | | |
| | 1-octylnonyl ester | | | |
| DSPC | 1,2-distearoyl-sn-glycero- | 816-94-4 | 10 | 0-35 |
| | 3-phosphatidylcholine | | | |
| Cholesterol | (3β)-cholest-5-en-3-ol | 57-88-5 | 38.5 | 0-55 |
| DMG- | α-[(2R)-2,3-bis[(1- | 160743- | 1.5 | 0-20 |
| PEG(2,000) | oxotetradecyl)oxy]propyl]- | 62-4 | | |
| | ω-methoxy-poly(oxy-1,2- | | | |
| | ethanediyl) | | | |

*SM-102 may be substituted for other fatty acids or fatty esters with similar hydrophobicity and molecular weight, but varying degree of saturation.

Possible variants of helper lipids include:

TABLE 5

| Lipid | | |
|---|---|---|
| ID | Formal Name | CAS # |
| DSPC | 1,2-distearoyl-sn-glycero-3-phosphatidylcholine | 816-94-4 |
| DOPC | 1,2-dioleoyl-sn-glycero-3-phosphatidylcholine | 4235-95-4 |
| SOPC | 1-stearoyl-2-oleoyl-sn-glycero-3-phosphocholine | 56421- |
| | | 10-4 |
| DOPE | 1,2-dioleoyl-sn-glycero-3-phosphatidylethanolamine | 4004-05-1 |
| DMPE | 1,2-Dimyristoyl-sn-glycero-3-Phosphoethanolamine | 998-07-2 |
| DPPE | 1,3-Dipalmitoyl glycero-2-Phosphoethanolamine | 67303- |
| | | 93-9 |
| DMPC | 1,2-Dimyristoyl-sn-glycero-3-phosphatidylcholine | 18194- |
| | | 24-6 |
| DPPC | 1,2-Dipalmitoyl-sn-glycero-3-phosphocholine | 63-89-8 |

Possible variants of PEGylated lipid include:

TABLE 6

| PEG lipid ID | Name | Details |
|---|---|---|
| DMG- | Dimyristoyl | 2,000 Da PEG chain with 2 |
| PEG(2,000) | PEG 2,000 | myristoyl (C14) anchors |
| DMG- | Dimyristoyl | 5,000 Da PEG chain with 2 |
| PEG(5,000) | PEG 5,000 | myristoyl (C14) anchors |
| DPPE- | Dipalmitoyl | 2,000 Da PEG chain with 2 |
| PEG(2,000) | PEG 2,000 | palmitoyl (C16) anchors |
| DPPE- | Dipalmitoyl | 5,000 Da PEG chain with 2 |
| PEG(5,000) | PEG 5,000 | palmitoyl (C16) anchors |
| DSG- | Dimyristoyl | 2,000 Da PEG chain with 2 |
| PEG(2,000) | PEG 2,000 | stearoyl (C18) anchors |
| DPPE- | Dimyristoyl | 5,000 Da PEG chain with 2 |
| PEG(5,000) | PEG 5,000 | stearoyl (C18) anchors |
| DOPE- | Dipalmitoyl | 2,000 Da PEG chain with 2 |
| PEG(2,000) | PEG 2,000 | oleoyl (C18) anchors |
| DPPC- | Dipalmitoyl | 5,000 Da PEG chain with 2 |
| PEG(5,000) | PEG 5,000 | oleoyl (C18) anchors |

Injection volume can range between 10 μL and 50 mL depending on disease, animal or human being treated, weight, or other physiological factors. For a reference formulation containing 5 ug of DNA, 141.6 ug of lipid is used as carrier in a 100 uL formulation.

Reference genetic constructs that may be used in the formulation or as reporter genes to evaluate gene expression, or as regulatory sequences in combination with reporter genes or therapeutic genes are listed in the table below:

TABLE 7

| | | | | | SEQ ID |
|---|---|---|---|---|---|
| # | Category | Example | Paste sequence | Use | NO: |
| 1 | Promoter | CMV | tagttattaatagtaatcaattacggggtcat | Drives | 1 |
| | | | tagttcatagcccatatatggagttccgcgtt | transgene or | |
| | | | acataacttacggtaaatggcccgcctggctg | suicide gene | |
| | | | accgcccaacgacccccgcccattgacgtcaa | | |
| | | | taatgacgtatgttcccatagtaacgccaata | | |
| | | | gggactttccattgacgtcaatgggtggagta | | |
| | | | tttacggtaaactgcccacttggcagtacatc | | |
| | | | aagtgtatcatatgccaagtacgccccctatt | | |
| | | | gacgtcaatgacggtaaatggcccgcctggca | | |
| | | | ttatgcccagtacatgaccttatgggactttc | | |
| | | | ctacttggcagtacatctacgtattagtcatc | | |
| | | | gctattaccatggtgatgcggttttggcagta | | |
| | | | catcaatgggcgtggatagcggtttgactcac | | |
| | | | ggggatttccaagtctccaccccattgacgtc | | |
| | | | aatgggagtttgttttggcaccaaaatcaacg | | |
| | | | ggactttccaaaatgtcgtaacaactccgccc | | |
| | | | cattgacgcaaatgggcggtaggcgtgtacgg | | |
| | | | tgggaggtctatataagcagagctggtttagt | | |
| | | | gaaccgtcagatc | | |
| 2 | | CBh | cgttacataacttacggtaaatggcccgcctg | Drives | 2 |
| | | | gctgaccgcccaacgacccccgcccattgacg | transgene or | |
| | | | tcaatagtaacgccaatagggactttccattg | suicide gene | |
| | | | acgtcaatgggtggagtatttacggtaaactg | | |
| | | | cccacttggcagtacatcaagtgtatcatatg | | |
| | | | ccaagtacgccccctattgacgtcaatgacgg | | |
| | | | taaatggcccgcctggcattgtgcccagtaca | | |
| | | | tgaccttatgggactttcctacttggcagtac | | |
| | | | atctacgtattagtcatcgctattaccatggt | | |
| | | | cgaggtgagccccacgttctgcttcactctcc | | |
| | | | ccatctcccccccctccccacccccaattttg | | |
| | | | tatttatttattttttaattattttgtgcagc | | |
| | | | gatgggggcggggggggggggggggcgcgcgc | | |
| | | | caggcggggcggggcggggcgaggggcggggc | | |
| | | | ggggcgaggcggagaggtgcggcggcagccaa | | |
| | | | tcagagcggcgcgctccgaaagtttcctttta | | |
| | | | tggcgaggcggcggcggcggcggccctataaa | | |
| | | | aagcgaagcgcgcggcgggcgggagtcgctgc | | |
| | | | gcgctgccttcgccccgtgccccgctccgccg | | |
| | | | ccgcctcgcgccgcccgccccggctctgactg | | |
| | | | accgcgttactcccacaggtgagcgggcggga | | |
| | | | cggcccttctcctccgggctgtaattagctga | | |
| | | | gcaagaggtaagggtttaagggatggttggtt | | |
| | | | ggtggggtattaatgtttaattacctggagca | | |
| | | | cctgcctgaaatcactttttttcaggttgg | | |
| 3 | | EFS | GGCTCCGGTGCCCGTCAGTGGGCAGAGCGCAC | Drives | 3 |
| | | | ATCGCCCACAGTCCCCGAGAAGITGGGGGGAG | transgene or | |
| | | | GGGTCGGCAATTGATCCGGTGCCTAGAGAAGG | suicide gene | |
| | | | TGGCGCGGGGTAAACTGGGAAAGTGATGTCGT | | |
| | | | GTACTGGCTCCGCCTTTTTCCCGAGGGTGGGG | | |
| | | | GAGAACCGTATATAAGTGCAGTAGTCGCCGTG | | |
| | | | AACGTTCTTTTTCGCAACGGGTTTGCCGCCAG | | |
| | | | AACACAGG | | |
| 4 | PRE | oPRE | gagcatcttaccgccatttatacccatatttg | Increses | 4 |
| | | | ttctgtttttcttgatttgggtatacatttaa | expression | |
| | | | atgttaataaaacaaaatggtggggcaatcat | level | |
| | | | ttacatttttagggatatgtaattactagttc | | |
| | | | aggtgtattgccacaagacaaacatgttaaga | | |
| | | | aactttcccgttatttacgctctgttcctgtt | | |
| | | | aatcaacctctggattacaaaatttgtgaaag | | |
| | | | attgactgatattcttaactatgttgctcctt | | |
| | | | ttacgctgtgtggatatgctgctttatagcct | | |
| | | | ctgtatctagctattgcttcccgtacggcttt | | |
| | | | cgttttctcctccttgtataaatcctggttgc | | |
| | | | tgtctcttttagaggagttgtggcccgttgtc | | |
| | | | cgtcaacgtggcgtggtgtgctctgtgtttgc | | |
| | | | tgacgcaacccccactggctggggcattgcca | | |
| | | | ccacctgtcaactcctttctgggactttcgct | | |
| | | | ttccccctcccgatcgccacggcagaactcat | | |
| | | | cgccgcctgccttgcccgctgctggacagggg | | |
| | | | ctaggttgctgggcactgataattccgtggtg | | |
| | | | ttgtc | | |
| 5 | | WPRE | cgataatcaacctctggattacaaaatttgtg | Increses | 5 |
| | | | aaagattgactggtattcttaactatgttgct | expression level | |
| | | | ccttttacgctatgtggatacgctgctttaat | | |
| | | | gcctttgtatcatgctattgcttcccgtatgg | | |
| | | | ctttcattttctcctccttgtataaatcctgg | | |
| | | | ttgctgtctctttatgaggagttgtggcccgt | | |
| | | | tgtcaggcaacgtggcgtggtgtgcactgtgt | | |
| | | | ttgctgacgcaacccccactggttggggcatt | | |
| | | | gccaccacctgtcagctcctttccgggacttt | | |
| | | | cgctttccccctccctattgccacggcggaac | | |
| | | | tcatcgccgcctgccttgcccgctgctggaca | | |
| | | | ggggctcggctgttgggcactgacaattccgt | | |
| | | | ggtgttgtcggggaagctgacgtcctttccat | | |
| | | | ggctgctcgcctgtgttgccacctggattctg | | |
| | | | cgcgggacgtccttctgctacgtcccttcggc | | |
| | | | cctcaatccagcggaccttccttcccgcggcc | | |
| | | | tgctgccggctctgcggcctcttccgcgtctt | | |
| | | | cgccttcgccctcagacgagtcggatctccct | | |
| | | | ttgggccgcctccccgcatcgg | | |
| 6 | DTS | SV40 DTS | GGTGTGGAAAGTCCCCAGGCTCCCCAGCAGGC | Promotes | 6 |
| | | | AGAAGTATGCAAAGCATGCATCTCAATTAGTC | nuclear | |
| | | | AGCAACCA | localization | |
| 7 | | 3NF1 | ctggggactttccagcctggggactttccagc | | 7 |
| | | | tgggactttccagg | | |
| 8 | | 3NF2 | ctggggactttccagctggggactttccagct | Promotes | 8 |
| | | | gggactttccaggag | nuclear | |
| | | | | localization | |
| 9 | Aptazyme | K19 | caaacaaacaaaggcgcgtcctggattcgtgg | Helps | 9 |
| | | | taaaacataccagatttcgatctggagaggtg | regulate | |
| | | | aagaatacgaccacctactacatccagctgat | expression of | |
| | | | gagtcccaaataggacgaaacgcgctcaaaca | down- | |
| | | | aacaaa | titration | |
| | | | | element | |
| 10 | CRISPRd | dSpCas9- | YMDKKYSIGLAIGTNSVGWAVITDEYKVPSKK | Promotes | 10 |
| | Cas Krab | ZN627 | FKVLGNTDRHSIKKNLIGALLFDSGETAEATR | down- | |
| | (amino | | LKRTARRRYTRRKNRICYLQEIFSNEMAKVDD | titration via | |
| | acid | | SFFHRLEESFLVEEDKKHERHPIFGNIVDEVA | epigenetic | |
| | sequence) | | YHEKYPTIYHLRKKLVDSTDKADLRLIYLALA | silencing | |
| | | | HMIKFRGHFLIEGDLNPDNSDVDKLFIQLVQT | | |
| | | | YNQLFEENPINASGVDAKAILSARLSKSRRLE | | |
| | | | NLIAQLPGEKKNGLFGNLIALSLGLTPNFKSN | | |
| | | | FDLAEDAKLQLSKDTYDDDLDNLLAQIGDQYA | | |
| | | | DLFLAAKNLSDAILLSDILRVNTEITKAPLSA | | |
| | | | SMIKRYDEHHQDLTLLKALVRQQLPEKYKEIF | | |
| | | | FDQSKNGYAGYIDGGASQEEFYKFIKPILEKM | | |
| | | | DGTEELLVKLNREDLLRKQRTFDNGSIPHQIH | | |
| | | | LGELHAILRRQEDFYPFLKDNREKIEKILTER | | |
| | | | IPYYVGPLARGNSRFAWMTRKSEETITPWNFE | | |
| | | | EVVDKGASAQSFIERMTNFDKNLPNEKVLPKH | | |
| | | | SLLYEYFTVYNELTKVKYVTEGMRKPAFLSGE | | |
| | | | QKKAIVDLLFKTNRKVTVKQLKEDYFKKIECF | | |
| | | | DSVEISGVEDRFNASLGTYHDLLKIIKDKDFL | | |
| | | | DNEENEDILEDIVLTLTLFEDREMIEERLKTY | | |
| | | | AHLFDDKVMKQLKRRRYTGWGRLSRKLINGIR | | |
| | | | DKQSGKTILDFLKSDGFANRNFMQLIHDDSLT | | |
| | | | FKEDIQKAQVSGQGDSLHEHIANLAGSPAIKK | | |
| | | | GILQTVKVVDELVKVMGRHKPENIVIEMAREN | | |
| | | | QTTQKGQKNSRERMKRIEEGIKELGSQILKEH | | |
| | | | PVENTQLQNEKLYLYYLQNGRDMYVDQELDIN | | |
| | | | RLSDYDVDAIVPQSFLKDDSIDNKVLIRSDKN | | |
| | | | RGKSDNVPSEEVVKKMKNYWRQLLNAKLITQR | | |
| | | | KFDNLTKAERGGLSELDKAGFIKRQLVETRQI | | |
| | | | TKHVAQILDSRMNTKYDENDKLIREVKVITLK | | |
| | | | SKLVSDFRKDFQFYKVREINNYHHAHDAYLNA | | |
| | | | VVGTALIKKYPKLESEFVYGDYKVYDVRKMIA | | |
| | | | KSEQEIGKATAKYFFYSNIMNFFKTEITLANG | | |
| | | | EIRKRPLIEINGETGEIVWDKGRDFATVRKVL | | |
| | | | SMPQVNIVKKTEVQTGGFSKESILPKRNSDKL | | |
| | | | IARKKDWDPKKYGGFDSPTVAYSVLVVAKVEK | | |
| | | | GKSKKLKSVKELLGITIMERSSFEKNPIDFLE | | |
| | | | AKGYKEVKKDLIIKLPKYSLFELENGRKRMLA | | |
| | | | SAGELQKGNELALPSKYVNFLYLASHYEKLKG | | |
| | | | SPEDNEQKQLFVEQHKHYLDEIIEQISEFSKR | | |
| | | | VILADANLDKVLSAYNKHRDKPIREQAENIIH | | |
| | | | LFTLTNLGAPAAFKYFDTTIDRKRYTSTKEVL | | |
| | | | DATLIHQSITGLYETRIDLSQLGGDPKKKRKV | | |
| | | | SGSETPGTSESATPESTGDSVAFEDVAVNFTL | | |
| | | | EEWALLDPSQKNLYRDVMRETFRNLASVGKQW | | |
| | | | EDQNIEDPFKIPRRNISHIPERLCESKEGGQG | | |
| | | | EE | | |
| 11 | Cre-Lox | Cre-ERT2 | MSNLLTVHQNLPALPVDATSDEVRKNLMDMFR | Enables down | 11 |
| | (amino | | DRQAFSEHTWKMLLSVCRSWAAWCKLNNRKWF | titration | |
| | acid | | PAEPEDVRDYLLYLQARGLAVKTIQQHLGQLN | | |
| | sequence) | | MLHRRSGLPRPSDSNAVSLVMRRIRKENVDAG | | |
| | | | ERAKQALAFERTDFDQVRSLMENSDRCQDIRN | | |
| | | | LAFLGIAYNTLLRIAEIARIRVKDISRTDGGR | | |
| | | | MLIHIGRTKTLVSTAGVEKALSLGVTKLVERW | | |
| | | | ISVSGVADDPNNYLFCRVRKNGVAAPSATSQL | | |
| | | | STRALEGIFEATHRLIYGAKDDSGQRYLAWSG | | |
| | | | HSARVGAARDMARAGVSIPEIMQAGGWINVNI | | |
| | | | VMNYIRNLDSETGAMVRLLEDGDLEPSAGDMR | | |
| | | | AANLWPSPLMIKRSKKNSLALSLTADQMVSAL | | |
| | | | LDAEPPILYSEYDPTRPFSEASMMGLLINLAD | | |
| | | | RELVHMINWAKRVPGFVDLTLHDQVHLLECAW | | |
| | | | LEILMIGLVWRSMEHPVKLLFAPNLLLDRNQG | | |
| | | | KCVEGMVEIFDMLLATSSRFRMMNLQGEEFVC | | |
| | | | LKSIILLNSGVYTFLSSTLKSLEEKDHIHRVL | | |
| | | | DKITDTLIHLMAKAGLTLQQQHQRLAQLLLIL | | |
| | | | SHIRHMSNKGMEHLYSMKCKNVVPLYDLLLEA | | |
| | | | ADAHRLHAPTSRGGASVEETDQSHLATAGSTS | | |
| | | | SHSLQKYYITGEAEGFPATA | | |
| 12 | Zn-Finger | hD3A- | MAPKKKRKVNHDQEFDPPKVYPPVPAEKRKPI | Promotes | 12 |
| | mediated | mD3L-ZF- | RVLSLFDGIATGLLVLKDLGIQVDRYIASEVC | down- | |
| | Epigenetic | ZN627 | EDSITVGMVRHQGKIMYVGDVRSVTQKHIQEW | titration via | |
| | silencer | | GPFDLVIGGSPCNDLSIVNPARKGLYEGTGRL | epigenetic | |
| | (amino | | FFEFYRLLHDARPKEGDDRPFFWLFENVVAMG | silencing | |
| | acid | | VSDKRDISRFLESNPVMIDAKEVSAAHRARYF | | |
| | sequence) | | WGNLPGMNRPLASTVNDKLELQECLEHGRIAK | | |
| | | | FSKVRTITTRSNSIKQGKDQHFPVFMNEKEDI | | |
| | | | LWCTEMERVFGFPVHYTDVSNMSRLARQRLLG | | |
| | | | RSWSVPVIRHLFAPLKEYFACVSSGNSNANSR | | |
| | | | GPSFSSGLVPLSLRGSHMGPMEIYKTVSAWKR | | |
| | | | QPVRVLSLFRNIDKVLKSLGFLESGSGSGGGT | | |
| | | | LKYVEDVTNVVRRDVEKWGPFDLVYGSTQPLG | | |
| | | | SSCDRCPGWYMFQFHRILQYALPRQESQRPFF | | |
| | | | WIFMDNLLLTEDDQETTTRFLQTEAVTLQDVR | | |
| | | | GRDYQNAMRVWSNIPGLKSKHAPLTPKEEEYL | | |
| | | | QAQVRSRSKLDAPKVDLLVKNCLLPLREYFKY | | |
| | | | FSQNSLPLSGGGGSGGGGSLEPGEKPYKCPEC | | |
| | | | GKSFSDPGHLVRHQRTHTGEKPYKCPECGKSF | | |
| | | | SDCRDLARHQRTHTGEKPYKCPECGKSFSSPA | | |
| | | | DLTRHQRTHTGEKPYKCPECGKSFSRADNLTE | | |
| | | | HQRTHTGEKPYKCPECGKSFSQSGHLTEHQRT | | |
| | | | HTGEKPYKCPECGKSFSRSDHLTTHQRTHTGK | | |
| | | | KISSGGGGSGGGGSDSVAFEDVAVNFTLEEWA | | |
| | | | LLDPSQKNLYRDVMRETFRNLASVGKQWEDQN | | |
| | | | IEDPFKIPRRNISHIPERLCESKEGGQGEE* | | |
| 13 | RapaCasp | RapaCasp | MASRILWHEMWHEGLEEASRLYFGERNVKGMF | Enables down | 13 |
| | (amino | 9 | EVLEPLHAMMERGPQTLKETSENQAYGRDLME | titration | |
| | acid | | AQEWCRKYMKSGNVKDLLQAWDLYYHVERRIS | | |
| | sequence) | | KLEYSGGGSLEGVQVETISPGDGRTFPKRGQT | | |
| | | | CVVHYTGMLEDGKKFDSSRDRNKPFKFMLGKQ | | |
| | | | EVIRGWEEGVAQMSVGQRAKLTISPDYAYGAT | | |
| | | | GHPGIIPPHATLVFDVELLKLESGGGGSGGGG | | |
| | | | SGGGGSGVDGFGDVGALESLRGNADLAYILSM | | |
| | | | EPCGHCLIINNVNFCRESGLRTRTGSNIDCEK | | |
| | | | LRRRFSSLHFMVEVKGDLTAKKMVLALLELAQ | | |
| | | | QDHGALDCCVVVILSHGCQASHLQFPGAVYGT | | |
| | | | DGCPVSVEKIVNIFNGTSCPSLGGKPKLFFIQ | | |
| | | | ACGGEQKDHGFEVASTSPEDESPGSNPEPDAT | | |
| | | | PFQEGLRTFDQLDAISSLPTPSDIFVSYSTFP | | |
| | | | GFVSWRDPKSGSWYVETLDDIFEQWAHSEDLQ | | |
| | | | SLLLRVANAVSVKGIYKQMPGCFNFLRKKLFF | | |
| | | | KTSAS | | |

One embodiment includes a formulation containing an adjustable therapeutic gene therapy system intended for the delivery to the subcutaneous space, and specifically one or more cell types in the subcutaneous space, which is comprised of a genetic construct encoding at least one therapeutic gene, under the regulation of at least one promoter element and optionally one or more regulatory elements, and a delivery carrier or vector encapsulating at least part of the said genetic construct, and a means of increasing the level of therapeutic transgene expression and/or a means for reducing (e.g, permanently) the level of therapeutic transgene expression (e.g., via application of an external stimulus). The delivery carrier or vector encapsulating at least a part of the said genetic construct may be of low immunogenicity, minimally immunogenic, or non-immunogenic, such that the same formulation, or elements of the same formulation can be repeatedly administered to the same organism without eliciting a neutralizing immune response that will decrease efficiency of the readministered dose by more than approximately 10%. In another embodiment the delivery carrier or vector is non-immunogenic allowing for the efficiency of the readministered dose to remain essentially or substantially the same as the originally delivered dose. In another embodiment this is regardless of the timeframe of the subsequent dose administration. In some embodiments the construct may carry at least one coding or non-coding therapeutic gene of interest encoding at least one functional part of a human peptide or protein or at least one functional part of a human peptide or protein analog or antagonist or at least one functional part of a non-human peptide or protein or at least one functional part of a non-human peptide or protein analog or antagonist. In some embodiments, the therapeutic gene encoded by the said gene therapy system is insulin, or a furin-cleavable insulin, or a modified insulin analog, or an insulin receptor agonist, or a Glucagon-Like Peptide-1 (GLP-1) peptide, GLP-1 receptor agonist, or a GLP-1 agonist analog. In another embodiment the therapeutic transgene encodes a gastric inhibitory peptide (GIP) or gastric inhibitory peptide analog, a GLP-1/GIP dual receptor agonist, co-agonist, or analog thereof. In another embodiment the therapeutic gene of interest is one or more of a growth factor, a cytokine, an anti-inflammatory protein, a pro-inflammatory protein, a complement protein, a receptor agonist or antagonist, a hormone, a peptide, a fusion protein comprised of one or more functional elements of different proteins, an immunogen, a vaccine, or a sub-unit or combination thereof.

In some embodiments the therapeutic gene therapy is for the delivery to the hypodermal layer, or generally the subcutaneous space, or the layer of tissue between the superficial and deep fascia, or any tissue containing adipocytes. Preferably, the gene therapy can all for up- or down-titration by means, such as other than via the systemic or local administration of peptides, hormones, small molecules, or proteins or other chemical entities. In some embodiments, the gene therapy is delivered to the subcutaneous fat pad.

In some embodiments the therapeutic gene therapy is formulated to deliver one or more of circular single-stranded DNA constructs, or circular double-stranded DNA constructs, or linear single-stranded DNA constructs, or linear double-stranded DNA constructs. In other embodiments the genetic cargo may be generally comprised of one or more of DNA, RNA, or a DNA-RNA hybrid, or a chemical derivative thereof. The genetic sequence of said construct may contain at least one promoter, which may be comprised of one or more of a constitutive promoter, an inducible promoter, a tissue specific promoter, or more specifically, CAG, EF1a, UBC, CBh, MSCV, hPGK, SFFV, or the SV40 promoter or combination thereof. In other embodiments the promoter may be a tetOn inducible promoter construct, or a tetOff promoter construct, and may optionally contain one or more of an enhancer, a regulator, an operator, or a repressor. In other embodiments the promoter element may an inducible promoter that can up- or down-regulate expression in response to external or internal stimuli such as inflammation, heat, light, stress, administration of steroids, tetracycline, antibiotics, rapamycin, ganciclovir, acyclovir, or is generally inducible by up- or down-regulated ROS, NOS, or cytokine release. In other embodiments, the promoter may respond to a circadian rhythm or be a cycling promoter that changes its level of activity by at least 5% with some periodicity ranging from hours to months. The gene therapy construct may contain one or more post translational regulatory elements including the Woodchuck Hepatitis Virus Posttranscriptional Regulatory Element or WPRE, or an optimized Woodchuck Hepatitis Virus Posttranscriptional Regulatory Element or WPRE, or a cis-acting element that can increase accumulation of cytoplasmic mRNA, or at least a part of the HIV type 1 Rev-Rev-responsive element, or at least a part of the human hepatitis virus post-translational regulatory element, or at least a part of one or more viral post-translational regulatory elements capable of improving expression of the gene of interest or any other construct-encoded elements. The gene therapy construct may contain one or more functional genetic sequences, which may promote translocation of at least 4% of the gene construct to the nucleus of target cells containing the therapeutic construct. In some embodiments the therapeutic construct can contain a protein coding sequence with a dedicated promoter, which encodes a protein to transport the therapeutic genetic construct to the nucleus or encode functional genetic sequences that promote translocation of at least 4% of the gene construct to the nucleus optionally encoding a sequence-specific DNA binding protein coupled to a nuclear localization signal peptide. Other means of translocating the gene therapy construct to the nucleus may include the encoding of a DNA nuclear target sequence recognized by one or more specific transcription factors, or a DNA nuclear target sequence (DTS) active in the importin-mediated transport system, or at least a part of the SV40 DTS, or at least a part of the glucocorticoid response element (GRE) DTS, or at least a part of the Sox2 regulatory region 2 DTS sequence, or at least a part of the importin beta (1), importin 7, NF-kappaBeta, or the small guanosine triphosphatase Ran interacting sequence.

The gene therapy as provided herein may be down-titrated by a physical, chemical, or physiological stimulus. In some embodiments the down-titration is facilitated by a functional genetic sequence or genetic element within the gene therapy construct, such elements may be inducible by one or more chemical, physical, or physiological stimuli and may be one or more of an inducible suicide gene, an inducible expression system encoding an RNA molecule capable of decreasing expression of at least a portion of the therapeutic genetic construct, a protein coding gene under the regulation of an inducible promoter, encoding a protein capable of targeted epigenetic silencing of at least a portion of the therapeutic genetic construct, or more specifically one or more of an HSV-TK suicide gene system, a RapaCas9 suicide gene system, or one or more of an miRNA, siRNA, shRNA, dsRNA, ncRNA, lncRNA, piwi-interacting RNA, PAT, eRNA, circRNA. In other embodiments the therapeutic gene construct may encode at least a guide RNA and CRISPR-dCas system coupled to a protein capable of inducing epigenetic silencing, or a guide RNA and CRISPR-dCas9-KRAB, or a guide RNA and CRISPR-dCas12b-KRAB, or a guide RNA and CRISPR-dCas8c-KRAB, or a guide RNA and CRISPR-dCas8a-KRAB, or a guide RNA and CRISPR-dCas8b-KRAB, or a TALEN, meganuclease, endonuclease, restriction enzyme, Zinc-Finger protein, or other DNA- or RNA-binding protein, or a combination thereof. The elements regulating expression, whether for up or down titration, or for permanent or transient regulation may include elements capable of changing the expression of one or more therapeutic transgenes in response to temperature, or generally heat, cold, thermal flux, ultrasound, focused energy, electromagnetic radiation, impulse, mechanical pressure, decrease in pressure, vacuum, suction, vibration, agitation, electrical stimuli, chemical stimuli, changes in physiological environment, changes in the level, concentration, gradient, or activity of paracrine, endocrine, or autocrine factors, changes in the level of local inflammation, or presence or absence of inflammatory cells or molecules, or a combination thereof. In some embodiments the gene therapy can be down-titrated by removing the cells via liposuction, microliposuction, or a microsurgical procedure, or through administration of ionizing or non-ionizing radiation. Regulatory elements may include genetic sequences that changes structure in response to internal or external stimulation, aptamers, aptazymes (such as for example the K19 aptazyme), protein binding sequences (such as histone binding sequences), sequences expressing ribosomes, RNA, protein, or RNA-protein complexes, or a combination thereof. Aforementioned regulatory elements may include at least a segment of a proximal promoter, distal promoter, insulator, or a combination thereof and positioned to regulate the therapeutic gene, dose adjustment mechanism, both, or other segments of the expression system. The aforementioned regulatory elements may be capable of forming secondary structures with themselves, other genomic sequences, or other sequences within the therapeutic construct. These secondary structures can serve as means to regulate expression, stability, transport of the construct to the nucleus or a combination thereof.

In some embodiments the genetic construct may be encased in a delivery carrier or vector, which may be a vehicle, capsule, envelope, capsid, a structural element, or a combination of one or more structure or structural elements. Said delivery carrier or vector may partially or completely envelope one or more genetic constructs and may be functionalized on the exterior, interior, or multiple surfaces. Said delivery carrier or vector may be comprised at least in part of lipid nanoparticles, liposomes, micelles, cubosomes, functionalized lipid nanoparticles, functionalized liposomes, stealth liposomes, solid lipid nanoparticles, lipid-polymer hybrid nanoparticle, transfectosomes, endosomes, exosomes, cell membrane, vesicular system, polymer, polymeric nanoparticles, non-immunogenic or hypoimmunogenic viral vectors, proteins or polypeptides, or combination thereof. One or more elements of the delivery carrier or vector may be functionalized with one or more functional elements to reduce immunogenicity, promote uptake by one or more specific cell types, promote uptake, endocytosis, pinocytosis, or enhance transport to the nucleus, or a combination thereof, or generally to optimize physical, physiological, or chemical activity or stability. In some embodiments, the gene therapy can be delivered inside cell membranes, or inside cellular organelles, or inside at least a portion of a cell. In other embodiments the genetic cargo can be delivered in a cell or as part of a cell therapy that is administered sub-cutaneously and has the potential for re-dosing and/or up- or down-titration. The delivery carrier or vector may contain one or more of an ionizable lipid, an ionizable element, an ionizable polymer, or an ionizable oligomer, or monomer, and may also optionally contain cationic or anionic elements, cholesterol, one or more amphiphilic polymer, polyethylene glycol or derivative, one or more antibody, nanobody, or antibody fragment, peptide or derivative, DNA or derivative, RNA or derivative, or other chemical or physical element or combination thereof. Some embodiments include a delivery carrier or vector with at least a part of the carrier or vector comprising a nanoparticle or a nanostructure being chemically or physically conjugated with peptides, proteins, or functional sequences derived from peptides or proteins. In other embodiments the delivery vector may be a nanoemulsion, nanostructured lipid, or comprised at least in part of an amphiphilic polymer or oligomer. In other embodiments the delivery carrier or vector may be comprised at least in part of a phospholipid, sphingolipid, ionizable lipid, polyelectrolytic polymer, polyelectrolytic complex, or cholesterol. And in other embodiments the delivery carrier or vector may be comprised at least in part of a metallic or ceramic nanoparticle. Embodiments of any one of the compositions or methods provided herein may entail the use of one or more delivery carriers or vectors, to deliver one or more genetic constructs with the goal of eliciting no humoral response or no cell mediated immune response, no memory immune response, or a minimal immune response that allows for re-dosing of the therapeutic formulation. The redosing efficiency following initial administration may be no less than 10% at the initial dose level, and elicit an immune response that does not result in severe adverse reactions. The delivery carrier or vector intended for the proposed gene therapy may be designed as optimally or minimally immunogenic such that any memory immune response generated by the carrier or vector or gene therapy is incapable of neutralizing the formulation to reduce delivery efficiency by more than 90%, or ideally 50%, and most ideally 25% or 1%, such that the efficiency of the subsequent dose is at least 10%, or ideally 50%, or most ideally 75% or 99% or some value within the aforementioned efficiencies. As such, some embodiments provide a delivery carrier or vector that is minimally immunogenic such that the memory immune response generated by the carrier or vector is incapable of neutralizing at least 50%, at least 75%, at least 90%, or at least 99% of a subsequently re-administered therapeutic formulation or a substantially similar therapeutic formulation and/or via substantially the same route of administration. In some embodiments, the therapeutic gene therapy construct can be down-titrated using an external stimulus, which optionally elicits one or more of a cooling of cutaneous and sub-cutaneous tissues to cause adipocyte cell death, cooling of cutaneous and sub-cutaneous tissues to cause adipocyte senescence, a change in temperature of the sub-cutaneous tissues sufficient to change expression of the therapeutic transgene, or a change in the physical environment of the adipocytes such that this change induces a reduction in gene expression from the therapeutic transgene. Said external stimulus or stimuli may be comprised at least in part of high-frequency ultrasound, medium-frequency ultrasound, low-frequency ultrasound, or sound waves capable of suppressing expression in adipocytes, inducing sentence in adipocytes, or inducing adipocyte cell death in at least a part of the sub-cutaneous space, or electromagnetic waves capable of suppressing expression in adipocytes, inducing sentence in adipocytes, or inducing adipocyte cell death in at least a part of the sub-cutaneous space, or of electrical signals capable of suppressing expression in adipocytes, inducing sentence in adipocytes, or inducing adipocyte cell death in at least a part of the sub-cutaneous space. Said stimulus may be combined with or solely be of a chemical nature including for example the administration of a formulation containing one or more of a Rapamycin or its derivative, a Tetracycline or its derivative, a combination of Rapamycin and Tetracycline or their derivatives, Gancyclovir or its derivative, Doxycycline or its derivative, or generally one or more pharmacologically active small molecules. Alternatively, down-titration may be applied indirectly via the delivery of a formulation containing one or more peptides with or without conjugation, or one or more hormones or their analogs, or more specifically a formulation containing tamoxifen or its derivative, which may then act directly on the DNA or proteins expressed from the therapeutic formulation, or on receptors on the cells containing the genetic constructs, or via other secondary means. Alternative or in combination with other external stimuli, the down-titration may be achieved via administration of one or more agents that elicit an inflammatory response, a formulation containing one or more steroids, a formulation containing one or more antibodies with or without functionalization, a formulation containing one or more receptor ligands, a formulation containing one or more antibody fragments, a formulation containing one or more proteins, a formulation containing one or more fusion proteins, a formulation containing one or more RNA molecules, a formulation containing one or more lipids, a formulation containing one or more metabolites, administration of agents that elicit an anti-inflammatory or immunomodulatory response, administration of a formulation containing one or more neurotransmitters, or combinations thereof. In some embodiments the external stimulus can be enacted or comprised of the administration of a formulation containing one or more proteins capable of directly or indirectly interacting with a promoter, RNA or DNA sequence. In yet other embodiments the external stimulus can be enacted or comprised of the administration of a formulation containing one or more RNA or DNA molecules capable of directly or indirectly interacting with a promoter, RNA or DNA sequence.

The gene therapy constructs disclosed herein can have the ability to increase the dose or up-titrate the treatment following initial administration. This up-titration can be permanent or transient and can be derived from an increase in transgene expression from the original dose or from additional doses of the gene therapy providing an absolute or relative increase over the original dose. In some embodiments the dose increase or up-titration is obtained via the administration of a second dose of the adjustable gene therapy, which is at least 1% of the original dose, or the administration of a second dose of the adjustable gene therapy, which is at least 2% of the original dose, or the administration of a second dose which is at least 5%, 10%, 15%, or 25% of the original dose. In other, embodiments, the increase in therapeutic transgene expression is achieved via the administration of an additional dose or doses of the gene therapy system. In other embodiments the means of increasing the therapeutic transgene expression or up-titration can be achieved by administration a formulation containing at least DNA, RNA, protein or combination thereof capable of transiently or permanently increasing therapeutic transgene expression. The subsequent administration of the therapeutic gene therapy can be performed in part or in whole to achieve up-titration, as such, the means of increasing the transgene expression can be comprised of administering at least 10% of at least one of the elements of any one of the gene therapy systems provided herein. In some embodiments the up-titration or increase of therapeutic transgene expression can be achieved via the administration of a small molecule capable of permanently or transiently increasing gene expression, or via the administration of an external stimulus such as heat, cold, electromagnetic radiation, ultrasound, sound waves, pressure, electrical stimulation, or other chemical or physical means to increase expression of the therapeutic transgene or reduce expression of an element that is capable of suppressing expression of the therapeutic transgene.

Increases or decreases in gene expression from the therapeutic construct may be transient or permanent and may comprised in the case of decreases: less than 25%, 40%, 60%, 80% or 90% of the total expression of the administered therapeutic, and in the case of increases more than 10%, 20%, 50%, or 100% of the total expression. In some embodiments permanent reduction can be complete or nearly complete, which is greater than 90% of the total expression of the administered therapeutic gene therapy and in other cases it can be regional, local, or impact only a part of the delivered genes separated by construct type, response element, anatomical area, expressed transgene or any other element differentiating the said parts.

An additional optional element is the use of markers to identify the position of the treatment. Said markers can generally by divided into generally superficial (defined as observable from the surface with minimal or without intervention) and/or internal (defined as observable from the surface following intervention). The markers may be used for identifying one or more of the type, position, dose, formulation, date of administration, or any other relevant information about the treatment or aspects relating to the treatment. Said markers may have many functions but may be generally used to locate the treatment for subsequent examination, up-titration site selection, down-titration of initial or other treatment, injection site selection for medication, administration of an inducer agent or administration of a stimulus to affect some change in the chemical, physical, or physiological environment of the therapy or prevent affecting a change in the chemical, physical, or physiological environment of the therapy. The markers may also be used to store information, save information, transmit information, or interact with information that is in some way relevant to the treatment.

Said markers (e.g., superficial markers) may be comprised of one or more of skin tattoos, skin marks, skin tags or means of marking the skin with information and be as simple as one round dot to as complex as a barcode, 2D data matrix, or QR-code. Said marks may be permanent or semi-permanent lasting days, weeks, months, or years. The marks may be visualized directly in daylight or visualizable under ultraviolet light or light in any other spectra range, may be fluorescent and require initial excitation prior to visualization, or may be visualizable by initial superficial administration of energy in the electromagnetic spectrum, ultrasound, or mechanical energy such as for example palpation.

Said markers (e.g., internal markers) may be delivered alongside the formulation, may be encoded into the genetic constructs in the formulation or as part of the formulation, or maybe administered following injection of the formulation. The aforementioned markers may be comprised of one or more of tantalum, gold, platinum, barium sulphate, or other metal or metal salt, with or without a carrier comprised at least in part of silicone, polyvinyl alcohol, or other biostable or biodegradable polymer. In yet other embodiments the markers may be at least partially comprised of a ceramic or composite material. Said markers may be passive such as a radiopaque material or actively emitting a signal, or present in an off or on state, which can be switched to the opposite state or another state following the application of a stimulus. The markers may be radio-opaque, opaque or semi-opaque to ultrasound, or other detection or measurement technique typically used for visualization of body or body structures. The markers may be atomic, molecular, or nanostructured, and may comprise a part of the formulation. In another embodiment, the markers may be encoded into the genetic construct which is delivered as part of the formulation. As an example, the marker may encode a fluorescent protein, that is expressed by the cell alongside the therapeutic transgene and said expression may be permanent, cyclical, or activated by an external stimulus such as heat, cold, radiation, light, administration of energy, or administration of one or more molecular compounds systemically or to the general area of the original treatment such as for example one or more of a tetracycline or derivative, a rapamycin or derivative, a ganciclovir or derivative, a steroid or derivative, a tamoxifen or derivative, or other therapeutic or molecular compound or formulation or combination thereof. In yet another embodiment, the marker may be a bioluminescent protein such as for example nano-luciferase or firefly luciferase, or any other bioluminescent protein, which can be constitutively luminescent in the body or luminescent following administration of a substrate, such as for example furimazine or a furimazine derivative or any other small molecule, metal, polymer, or ceramic substrate for the encoded marker enzyme. Said reporter genes may be expressed constitutively or following administration of a physical or chemical stimulus such as for example heat or tetracycline, or generally energy in the electromagnetic spectrum, ultrasound, heat, cold, or any pharmacologically or chemically active substance.

The said gene therapy is intended to treat human or veterinary diseases, which in some embodiments can be categorized as treatments and in other embodiments as augmentation or enhancements of human or animal health, physical condition, mental state, or physical or mental abilities. In yet other embodiments the gene therapy can be used to increase or modify the human or animal longevity, healthspan, or the longevity or healthspan of a specific tissue, organ, cell or combination thereof.

In some embodiments the target cell type is one or more of an adipocyte, pre-adipocyte, progenitor capable of differentiating into an adipocyte, stem cell capable of differentiating into an adipocyte, a resident cell type of the subcutaneous tissues, or a transient cell or stem cell, but one that can become a resident cell of the subcutaneous tissues. The gene therapy is intended to treat or prevent any human or veterinary disorder, but may be used in some specific embodiments to treat monogenic disorders, enzyme insufficiency disorders, protein insufficiency disorders (as protein replacement therapy), metabolic disorders, autoimmune disorders, oncologies, neurologic disorders, cardiovascular pathologies, musculoskeletal disorders, hematologic disorders, infectious disorders, dermatologic disorders, immune system disorders, pulmonary system disorders, kidney or bladder disorders, or disorders of other organs, tissues, systems, or combinations thereof. In embodiments, the therapeutic gene therapy system is intended for the delivery of therapeutic or prophylactic antibodies via expression from the genetic construct, therapeutic, preventative, augmentative, or prophylacitic proteins, fusion proteins, peptides, immunogens, antigens, enzymes, ribozymes, mRNA, non-coding RNA, miRNA, shRNA, or other RNA, or molecules that can act as vaccines or adjuvants via expression from the genetic construct.

The disclosed gene therapy treatment may be delivered in a vial for subsequent administration, or a prefilled syringe or cartridge. Alternatively, the treatment can be administered via an autoinjector or a patch injector, via ultrasonic guidance, or via other guidance techniques to ensure delivery to the correct location. Alternatively, and optionally the treatment can be delivered by a set of needles or via a set or series of injections to more evenly distribute the constructs in the treatment site. The gene therapy treatment can be delivered via a patch, microneedles, a set of microneedles, jet injection, on a carrier, or using other means to alter the integrity of the tissues through which or into which it is being delivered.

It should be understood that within the scope of the compositions or methods provided herein the formulation, materials, genetic constructs, sequences, biological and chemical compositions, markers or methods of use may be varied by one skilled in the art, to the extent that the structures described herewithin perform the desired function and remain within the scope of the compositions or methods provided herein. Various parts, components or characteristics may be used in combination, with or without modification by someone skilled in the art to achieve the desired functionality of the aforedescribed formulations.

Moreover, all individual features and methods of use described herein, and each and every combination of two or more of such features and methods of use, are included within the scope of the compositions or methods provided herein provided that these features and methods of use in such a combination are not mutually inconsistent. It is understood that certain portions or combinations of such portions can be varied by someone trained in the art while still achieving the goal(s) of the compositions or methods provided herein.

Finally, it is understood that the specific ranges provided herein are not restrictive and are for example purposes only.

### EXAMPLES

### Example 1

This example outlines a minimal plasmid construct, delivered within a Lipid Nanoparticle formulation that contains an inducible promoter, post-translational regulatory element, gene of interest (a GLP-1 receptor agonist), nuclear targeting element, and a genetic regulatory element that enables the reduction of gene of interest expression following administration of ganciclovir.

### Genetic Construct Map (Excluding Plasmid Backbone):

Full-length Construct Sequence (excluding plasmid backbone):
(SEQ ID NO: 14)
ctgcgcgctcgctcgctcactgaggccgcccgggcaaagcccgggcgtcgggcgacctttggtcgccc
ggcctcagtgagcgagcgagcgcgcagagagggagtggccaactccatcactaggggttcctatcgat
caactttgtatagaaaagttgctggggactttccagcctggggactttccagctgggactttccaggc
aactttgtatagaaaagttgggtaccgagctcgactttcacttttctctatcactgatagggagtggt
aaactcgactttcacttttctctatcactgatagggagtggtaaactcgactttcacttttctctatc
actgatagggagtggtaaactcgactttcacttttctctatcactgatagggagtggtaaactcgact
ttcacttttctctatcactgatagggagtggtaaactcgactttcacttttctctatcactgataggg
agtggtaaactcgactttcacttttctctatcactgatagggagtggtaaactcgacctatataagca
gagctcgtttagtgaaccgtcagatcgcctggagacgccatccacgctgttttgacctccatagaaga
caccgggaccgatccagcctccgcggccccgaattgcaagtttgtacaaaaaagcaggctgccaccat
gcacggcgagggcacattcaccagcgacctgagcaagcagatggaagaggaagccgtgagactgttca
tcgagtggctgaagaacggcggaccctccagcggcgcccctccttctaagaaaaagaaaaagaagtaa
acccagctttcttgtacaaagtggtgatggccggccgcttcgagcagacatgataagatacattgatg
agtttggacaaaccacaactagaatgcagtgaaaaaaatgctttatttgtgaaatttgtgatgctatt
gctttatttgtaaccattataagctgcaataaacaagttaacaacaacaattgcattcattttatgtt
tcaggttcagggggaggtgtgggaggttttttaaagcaagtaaaacctctacaaatgtggtaatcgat
ccatagagcccaccgcatccccagcatgcctgctattgtcttcccaatcctcccccttgctgtcctgc
cccaccccaccccccagaatagaatgacacctactcagacaatgcgatgcaatttcctcattttatta
ggaaaggacagtgggagtggcaccttccagggtcaaggaaggcacgggggaggggcaaacaacagatg
gctggcaactagaaggcacagttacccggggagcatgtcaaggtcaaaatcgtcaagagcgtcagcag
gcagcatatcaaggtcaaagtcgtcaagggcatcggctgggagcatgtctaagtcaaaatcgtcaagg
gcgtcggccggcccgccgctttcgcactttagctgtttctccaggccacatatgattagttccaggcc
gaaaaggaaggcaggttcggctccctgccggtcgaacagctcaattgcttgtctcagaagtgggggca
tagaatcggtggtaggtgtctctctttcctcttttgctacttgatgctcctgttcctccaatacgcag
cccagtgtaaagtggcccacggcggacagagcgtacagtgcgttctccagggagaagccttgctgaca
caggaacgcgagctgattttccagggtttcgtactgtttctctgttgggcgggtgccgagatgcactt
tagccccgtcgcgatgtgagaggagagcacagcggaatgacttggcgttgttccgcagaaagtcttgc
catgactcgccttccagggggcagaagtgggtatgatgcctgtccagcatctcgattggcagggcatc
gagcagggcccgcttgttcttcacgtgccagtacagggtaggctgctcaactcccagcttttgagcga
gtttccttgtcgtcaggccttcgataccgactccattgagtaattccagagcgccgtttatgactttg
ctcttgtccagtctagacatggggccgggattttcctccacgtccccgcatgttagaagacttcccct
gccctctccggagccccagggatcctctccttgctgcaacagggagatcacctttggtttggtaaaca
ggaatcctgccatggaggccaggttgctgtaattctccagcatcacctcacggtacaggcttctctga
gacagatccagcttcttccactcgtcccgagtaaagagcacagccacatcttcaaatgtcactgacac
tgctagctttctcttcttttttggtttattaccatcctcaatgggtgtatgctttttttcatctaact
gtgcagaaaatccaaatattaatgatttcagcccgaaatggaaggccatttcagcagaggttctcgcc
tgaatattaaatgcctcttgtaataatggtggtgcagcgtctatcacatgattattttctatctggtt
tgttgcttgctcctctaatactgcacccaacgtaaaatggctgatagattgcagaatgaaaagagcct
cctcgaccgaaaaccctgcatcgcatagacagcgtagttgcgcctctgcttgttcaaactgggggggc
gtaggagaggtccctatatgcaatcgggctccatcacgatggaccagtaatgctttacggaaactcag
agcattttcctggagaaactgctgccaactctcagtcggtaacggtgctgaacgggtgtgatgcttcg
ccagtattgcctctgaaagcatgttcataagagtctgcttgttgcgcacgtgccaatacaatgtaggc
tgctctacacctagcttctgggcgagtttacgggttgttaaaccttcgattccgacctcattaagcag
ctctaatgcgctgttaatcactttacttttatctaatctagacatgaattcccaacctgaaaaaaagt
gatttcaggcaggtgctccaggtaattaaacattaataccccaccaaccaaccatcccttaaaccctt
acctcttgctcagctaattacagcccggaggagaagggccgtcccgcccgctcacctgtgggagtaac
gcggtcagtcagagccggggcgggcggcgcgaggcggcggcggagcggggcacggggcgaaggcagcg
cgcagcgactcccgcccgccgcgcgcttcgctttttatagggccgccgccgccgccgcctcgccataa
aaggaaactttcggagcgcgccgctctgattggctgccgccgcacctctccgcctcgccccgccccgc
ccctcgccccgccccgccccgcctggcgcgcgccccccccccccccccgcccccatcgctgcacaaaa
taattaaaaaataaataaatacaaaattgggggtggggaggggggggagatggggagagtgaagcaga
acgtggggctcacctcgaccatggtaatagcgatgactaatacgtagatgtactgccaagtaggaaag
tcccataaggtcatgtactgggcacaatgccaggcgggccatttaccgtcattgacgtcaataggggg
cgtacttggcatatgatacacttgatgtactgccaagtgggcagtttaccgtaaatactccacccatt
gacgtcaatggaaagtccctattggcgttactattgacgtcaatgggcgggggtcgttgggcggtcag
ccaggcgggccatttaccgtaagttatgtaacggggccgcgggccgccgatcgaacttgtttattgca
gcttataatggttacaaataaagcaatagcatcacaaatttcacaaataaagcatttttttcactgca
ttctagttgtggtttgtccaaactcatcaatgtatcttatttgtttgtttgagcgcgtttcgtcctat
ttgggactcatcagctggatgtagtaggtggtcgtattcttcacctctccagatcgaaatctggtatg
ttttaccacgaatccaggacgcgcctttgtttgtttggactagaagcggccgcctattagttggcctc
tcccatttcccgggcgaatgttctggccaggtcgcagatggtagggatagatccaggtgtggtcacgt
gggtctggatcatgccgcttgtcagttgcagcagtgcatctctacatccggcaggagactggtcgtag
tccaggatgaacacgtgcataggcctcagtcttttagccagcacgtcgagggcccaggcaaacacgtt
gtacaggtcgccgttaggggccagcagctcaggggctctgaacagggtaaacagcgtatcgccgatgt
ggggtctagggccggcgttgctttgaggctcggcgccctgggggggcacggcggtcccgctcagctgg
ccccagtcctctctccagcttccgccgccctgcaggtatctcacggtattggccagcaggccatacac
tctgcggatagcagccagcatggccagatccagtctctcgcctggccgctgccgcttggccagtctat
cgatgtgtctatcttctggcagggcgcccagcacgatgtttgtgccaggcagggtgggggggatcagg
gccacgaaggccagcacggcctgaggtgtcatggagcccatcaggtatctagcagcggggtagcacag
cagagcggcgatggggtgccggtcgaagataagggtcagagctggaggaggggcgtggctgctaccgg
cttcgccgccaatatgaggagccagcacggcgtcggtgacggcgtagggcatgcccatggtgatctgg
gcgctggtcatcaccacggcggcgtcgccagcgctgatctcgccctgatccagtctgtgctgagtggt
gtagatattggcgatggtttcagaggctcccaggacccgccagtaggtcatgggctcaggcacgtaca
caatgtcgtctctggagcccagggccacgagcagctgtgttgtggtggtcttgcccatgccgtgaggg
ccgtcgatgtaaacccgcagcagtgtaggcatggtggcagcctgcttttttgtacaaacttgtcacga
cacctgaaatggaagaaaaaaactttgaaccactgtctgaggcttgagaatgaaccaagatccaaact
caaaaagggcaaattccaaggagaattacatcaagtgccaagctggcctaacttcagtctccacccac
tcagtgtggggaaactccatcgcataaaacccctccccccaacctaaagacgacgtactccaaaagct
cgagaactaatcgaggtgcctggacggcgcccggtactccgtggagtcacatgaagcgacggctgagg
acggaaaggcccttttcctttgtgtgggtgactcacccgcccgctctcccgagcgccgcgtcctccat
tttgagctccctgcagcagggccgggaagcggccatctttccgctcacgcaactggtgccgaccgggc
cagccttgccgcccagggcggggcgatacacggcggcgcgaggccaggcaccagagcaggccggccag
cttgagactacccccgtccgattctcggtggccgcgctcgcaggccccgcctcgccgaacatgtgcgc
tgggacgcacgggccccgtcgccgcccgcggccccaaaaaccgaaataccagtgtgcagatcttggcc
cgcatttacaagactatcttgccagaaaaaaagcgtcgcagcaggtcatcaaaaattttaaatggcta
gagacttatcgaaagcagcgagacaggcgcgaaggtgccaccagattcgcacgcggcggccccagcgc
ccaggccaggcctcaactcaagcacgaggcgaaggggctccttaagcgcaaggcctcgaactctccca
cccacttccaacccgaagctcgggatcaagaatcacgtactgcagccaggtggaagtaattcaaggca
cgcaagggccataacccgtaaagaggccaggcccgcgggaaccacacacggcacttacctgtgttctg
gcggcaaacccgttgcgaaaaagaacgttcacggcgactactgcacttatatacggttctcccccacc
ctcgggaaaaaggcggagccagtacacgacatcactttcccagtttaccccgcgccaccttctctagg
caccggttcaattgccgacccctccccccaacttctcggggactgtgggcgatgtgcgctctgcccac
tgacgggcaccggagcccaacttttctatacaaagttgtctagactggggactttccagctggggact
ttccagctgggactttccaggagagatctaggaacccctagtgatggagttggccactccctctctgc
gcgctcgctcgctcactgaggccgggcgaccaaaggtcgcccgacgcccgggctttgcccgggcggcc
tcagtgagcgagcgagcgcgcag
AAV2 ITR:
(SEQ ID NO: 15)
ctgcgcgctcgctcgctcactgaggccgcccgggcaaagcccgggcgtcgggcgacctttggtcgccc
ggcctcagtgagcgagcgagcgcgcagagagggagtggccaactccatcactaggggttcct
3NF1:
(SEQ ID NO: 7)
ctggggactttccagcctggggactttccagctgggactttccagg
3NF2:
(SEQ ID NO: 8)
Ctggggactttccagctggggactttccagctgggactttccaggag
TRE promoter:
(SEQ ID NO: 16)
Ggtaccgagctcgactttcacttttctctatcactgatagggagtggtaaactcgactttcacttttc
tctatcactgatagggagtggtaaactcgactttcacttttctctatcactgatagggagtggtaaac
tcgactttcacttttctctatcactgatagggagtggtaaactcgactttcacttttctctatcactg
atagggagtggtaaactcgactttcacttttctctatcactgatagggagtggtaaactcgactttca
cttttctctatcactgatagggagtggtaaactcgacctatataagcagagctcgtttagtgaaccgt
cagatcgcctggagacgccatccacgctgttttgacctccatagaagacaccgggaccgatccagcct
ccgcggccccgaattg
Kozak sequence:
(SEQ ID NO: 17)
gccaccatgg
GLP-1 receptor agonist (RA) Nucleotide Sequence:
(SEQ ID NO: 18)
atgcacggcgagggcacattcaccagcgacctgagcaagcagatggaagaggaagccgtgagactgtt
catcgagtggctgaagaacggcggaccctccagcggcgcccctccttctaagaaaaagaaaaagaagt
aa
GLP-1 RA Amino acid sequence:
(SEQ ID NO: 19)
MHGEGTFTSDLSKQMEEEAVRLFIEWLKNGGPSSGAPPSKKKKKK
SV40 late pA:
(SEQ ID NO: 20)
Cagacatgataagatacattgatgagtttggacaaaccacaactagaatgcagtgaaaaaaatgcttt
atttgtgaaatttgtgatgctattgctttatttgtaaccattataagctgcaataaacaagttaacaa
caacaattgcattcattttatgtttcaggttcagggggaggtgtgggaggttttttaaagcaagtaaa
acctctacaaatgtggta
CBh promoter:
(SEQ ID NO: 2)
cgttacataacttacggtaaatggcccgcctggctgaccgcccaacgacccccgcccattgacgtcaa
tagtaacgccaatagggactttccattgacgtcaatgggtggagtatttacggtaaactgcccacttg
gcagtacatcaagtgtatcatatgccaagtacgccccctattgacgtcaatgacggtaaatggcccgc
ctggcattgtgcccagtacatgaccttatgggactttcctacttggcagtacatctacgtattagtca
tcgctattaccatggtcgaggtgagccccacgttctgcttcactctccccatctcccccccctcccca
cccccaattttgtatttatttattttttaattattttgtgcagcgatgggggcggggggggggggggg
gcgcgcgccaggcggggcggggcggggcgaggggcgggcgggggcgaggcggagaggtgcggcggcag
ccaatcagagcggcgcgctccgaaagtttccttttatggcgaggcggcggcggcggcggccctataaa
aagcgaagcgcgcggcgggcgggagtcgctgcgcgctgccttcgccccgtgccccgctccgccgccgc
ctcgcgccgcccgccccggctctgactgaccgcgttactcccacaggtgagcgggcgggacggccctt
ctcctccgggctgtaattagctgagcaagaggtaagggtttaagggatggttggttggtggggtatta
atgtttaattacctggagcacctgcctgaaatcactttttttcaggttgg
tTS Nucleotide Sequence:
(SEQ ID NO: 21)
atgtctagattagataaaagtaaagtgattaacagcgcattagagctgcttaatgaggtcggaatcga
aggtttaacaacccgtaaactcgcccagaagctaggtgtagagcagcctacattgtattggcacgtgc
gcaacaagcagactcttatgaacatgctttcagaggcaatactggcgaagcatcacacccgttcagca
ccgttaccgactgagagttggcagcagtttctccaggaaaatgctctgagtttccgtaaagcattact
ggtccatcgtgatggagcccgattgcatatagggacctctcctacgcccccccagtttgaacaagcag
aggcgcaactacgctgtctatgcgatgcagggttttcggtcgaggaggctcttttcattctgcaatct
atcagccattttacgttgggtgcagtattagaggagcaagcaacaaaccagatagaaaataatcatgt
gatagacgctgcaccaccattattacaagaggcatttaatattcaggcgagaacctctgctgaaatgg
ccttccatttcgggctgaaatcattaatatttggattttctgcacagttagatgaaaaaaagcataca
cccattgaggatggtaataaaccaaaaaagaagagaaagctagcagtgtcagtgacatttgaagatgt
ggctgtgctctttactcgggacgagtggaagaagctggatctgtctcagagaagcctgtaccgtgagg
tgatgctggagaattacagcaacctggcctccatggcaggattcctgtttaccaaaccaaaggtgatc
tccctgttgcagcaaggagaggatccctgg
tTS Amino Acid Sequence:
(SEQ ID NO: 22)
MSRLDKSKVINSALELLNEVGIEGLTTRKLAQKLGVEQPTLYWHVRNKQTLMNMLSEAILAKHHTRSA
PLPTESWQQFLQENALSFRKALLVHRDGARLHIGTSPTPPQFEQAEAQLRCLCDAGFSVEEALFILQS
ISHFTLGAVLEEQATNQIENNHVIDAAPPLLQEAFNIQARTSAEMAFHFGLKSLIFGFSAQLDEKKHT
PIEDGNKPKKKRKLAVSVTFEDVAVLFTRDEWKKLDLSQRSLYREVMLENYSNLASMAGFLFTKPKVI
SLLQQGEDPW
T2A Linker Nucleotide Sequence:
ggctccgga
T2A Linker Amino Acid Sequence:
GSG
T2A Nucleotide Sequence:
(SEQ ID NO: 23)
gagggcaggggaagtcttctaacatgcggggacgtggaggaaaatcccggcccc
T2A Amino Acid Sequence:
(SEQ ID NO: 24)
EGRGSLLTCGDVEENPGP
rtTA Nucleotide Sequence:
(SEQ ID NO: 25)
atgtctagactggacaagagcaaagtcataaacggcgctctggaattactcaatggagtcggtatcga
aggcctgacgacaaggaaactcgctcaaaagctgggagttgagcagcctaccctgtactggcacgtga
agaacaagcgggccctgctcgatgccctgccaatcgagatgctggacaggcatcatacccacttctgc
cccctggaaggcgagtcatggcaagactttctgcggaacaacgccaagtcattccgctgtgctctcct
ctcacatcgcgacggggctaaagtgcatctcggcacccgcccaacagagaaacagtacgaaaccctgg
aaaatcagctcgcgttcctgtgtcagcaaggcttctccctggagaacgcactgtacgctctgtccgcc
gtgggccactttacactgggctgcgtattggaggaacaggagcatcaagtagcaaaagaggaaagaga
gacacctaccaccgattctatgcccccacttctgagacaagcaattgagctgttcgaccggcagggag
ccgaacctgccttccttttcggcctggaactaatcatatgtggcctggagaaacagctaaagtgcgaa
agcggcgggccggccgacgcccttgacgattttgacttagacatgctcccagccgatgcccttgacga
ctttgaccttgatatgctgcctgctgacgctcttgacgattttgaccttgacatgctccccgggtaa
rtTA Amino Acid Sequence:
(SEQ ID NO: 26)
MSRLDKSKVINGALELLNGVGIEGLTTRKLAQKLGVEQPTLYWHVKNKRALLDALPIEMLDRHHTHFC
PLEGESWQDFLRNNAKSFRCALLSHRDGAKVHLGTRPTEKQYETLENQLAFLCQQGFSLENALYALSA
VGIFTLGCVLEEQEHQVAKEERETPTTDSMPPLLRQAIELFDRQGAEPAFLFGLELIICGLEKQLKCE
SGGPADALDDFDLDMLPADALDDFDLDMLPADALDDEDLDMLPG
BGH pA:
(SEQ ID NO: 27)
ctgtgccttctagttgccagccatctgttgtttgcccctcccccgtgccttccttgaccctggaaggt
gccactcccactgtcctttcctaataaaatgaggaaattgcatcgcattgtctgagtaggtgtcattc
tattctggggggtggggtggggcaggacagcaagggggaggattgggaagacaatagcaggcatgctg
gggatgcggtgggctctatgg
EF1a promoter:
(SEQ ID NO: 28)
ggctccggtgcccgtcagtgggcagagcgcacatcgcccacagtccccgagaagttggggggaggggt
cggcaattgaaccggtgcctagagaaggtggcgcggggtaaactgggaaagtgatgtcgtgtactggc
tccgcctttttcccgagggtgggggagaaccgtatataagtgcagtagtcgccgtgaacgttcttttt
cgcaacgggtttgccgccagaacacaggtaagtgccgtgtgtggttcccgcgggcctggcctctttac
gggttatggcccttgcgtgccttgaattacttccacctggctgcagtacgtgattcttgatcccgagc
ttcgggttggaagtgggtgggagagttcgaggccttgcgcttaaggagccccttcgcctcgtgcttga
gttgaggcctggcctgggcgctggggccgccgcgtgcgaatctggtggcaccttcgcgcctgtctcgc
tgctttcgataagtctctagccatttaaaatttttgatgacctgctgcgacgctttttttctggcaag
atagtcttgtaaatgcgggccaagatctgcacactggtatttcggtttttggggccgcgggcggcgac
ggggcccgtgcgtcccagcgcacatgttcggcgaggcggggcctgcgagcgcggccaccgagaatcgg
acgggggtagtctcaagctggccggcctgctctggtgcctggcctcgcgccgccgtgtatcgccccgc
cctgggcggcaaggctggcccggtcggcaccagttgcgtgagcggaaagatggccgcttcccggccct
gctgcagggagctcaaaatggaggacgcggcgctcgggagagcgggcgggtgagtcacccacacaaag
gaaaagggcctttccgtcctcagccgtcgcttcatgtgactccacggagtaccgggcgccgtccaggc
acctcgattagttctcgagcttttggagtacgtcgtctttaggttggggggaggggttttatgcgatg
gagtttccccacactgagtgggtggagactgaagttaggccagcttggcacttgatgtaattctcctt
ggaatttgccctttttgagtttggatcttggttcattctcaagcctcagacagtggttcaaagttttt
ttcttccatttcaggtgtcgtga
HSV-TK sequence:
(SEQ ID NO: 29)
atgcctacactgctgcgggtttacatcgacggccctcacggcatgggcaagaccaccacaacacagct
gctcgtggccctgggctccagagacgacattgtgtacgtgcctgagcccatgacctactggcgggtcc
tgggagcctctgaaaccatcgccaatatctacaccactcagcacagactggatcagggcgagatcagc
gctggcgacgccgccgtggtgatgaccagcgcccagatcaccatgggcatgccctacgccgtcaccga
cgccgtgctggctcctcatattggcggcgaagccggtagcagccacgcccctcctccagctctgaccc
ttatcttcgaccggcaccccatcgccgctctgctgtgctaccccgctgctagatacctgatgggctcc
atgacacctcaggccgtgctggccttcgtggccctgatcccccccaccctgcctggcacaaacatcgt
gctgggcgccctgccagaagatagacacatcgatagactggccaagcggcagcggccaggcgagagac
tggatctggccatgctggctgctatccgcagagtgtatggcctgctggccaataccgtgagatacctg
cagggcggcggaagctggagagaggactggggccagctgagcgggaccgccgtgcccccccagggcgc
cgagcctcaaagcaacgccggccctagaccccacatcggcgatacgctgtttaccctgttcagagccc
ctgagctgctggcccctaacggcgacctgtacaacgtgtttgcctgggccctcgacgtgctggctaaa
agactgaggcctatgcacgtgttcatcctggactacgaccagtctcctgccggatgtagagatgcact
gctgcaactgacaagcggcatgatccagacccacgtgaccacacctggatctatccctaccatctgcg
acctggccagaacattcgcccgggaaatgggagaggccaactaa
HSV-TK amino acid sequence:
(SEQ ID NO: 30)
MPTLLRVYIDGPHGMGKTTTTQLLVALGSRDDIVYVPEPMTYWRVLGASETIANIYTTQHRLDQGEIS
AGDAAVVMTSAQITMGMPYAVTDAVLAPHIGGEAGSSHAPPPALTLIFDRHPIAALLCYPAARYLMGS
MTPQAVLAFVALIPPTLPGINIVLGALPEDRHIDRLAKRQRPGERLDLAMLAAIRRVYGLLANTVRYL
QGGGSWREDWGQLSGTAVPPQGAEPQSNAGPRPHIGDTLFTLFRAPELLAPNGDLYNVFAWALDVLAK
RLRPMHVFILDYDQSPAGCRDALLQLTSGMIQTHVTTPGSIPTICDLARTFAREMGEAN
Insulator:
(SEQ ID NO: 31)
caaacaaacaaa
K19 aptazyme:
(SEQ ID NO: 32)
ggcgcgtcctggattcgtggtaaaacataccagatttcgatctggagaggtgaagaatacgaccacct
actacatccagctgatgagtcccaaataggacgaaacgcgct
SV40 pA:
(SEQ ID NO: 33)
Taagatacattgatgagtttggacaaaccacaactagaatgcagtgaaaaaaatgctttatttgtgaa
atttgtgatgctattgctttatttgtaaccattataagctgcaataaacaagtt

### Example 2

This example outlines a hairpin DNA construct, delivered within a polymeric lipoplex that contains a constitutive promoter, post-translational regulatory element, gene of interest (furin-cleavable insulin), nuclear targeting element, and a genetic regulatory element that enables the reduction of gene of interest expression following administration of a steroid and a second induction agent.

### Genetic Construct Map:

Full-length Construct Sequence (excluding plasmid backbone):
(SEQ ID NO: 34)
ctgcgcgctcgctcgctcactgaggccgcccgggcaaagcccgggcgtcgggcgacctttggtcgccc
ggcctcagtgagcgagcgagcgcgcagagagggagtggccaactccatcactaggggttccttctaga
ggtgtggaaagtccccaggctccccagcaggcagaagtatgcaaagcatgcatctcaattagtcagca
accacaactttgtatagaaaagttgctagttattaatagtaatcaattacggggtcattagttcatag
cccatatatggagttccgcgttacataacttacggtaaatggcccgcctggctgaccgcccaacgacc
cccgcccattgacgtcaataatgacgtatgttcccatagtaacgccaatagggactttccattgacgt
caatgggtggagtatttacggtaaactgcccacttggcagtacatcaagtgtatcatatgccaagtac
gccccctattgacgtcaatgacggtaaatggcccgcctggcattatgcccagtacatgaccttatggg
actttcctacttggcagtacatctacgtattagtcatcgctattaccatggtcgaggtgagccccacg
ttctgcttcactctccccatctcccccccctccccacccccaattttgtatttatttattttttaatt
attttgtgcagcgatgggggcggggggggggggggcgcgcgccaggcggggcggggcggggcgagggg
cggggcggggcgaggcggagaggtgcggcggcagccaatcagagcggcgcgctccgaaagtttccttt
tatggcgaggcggcggcggcggcggccctataaaaagcgaagcgcgcggcgggcgggagtcgctgcgc
gctgccttcgccccgtgccccgctccgccgccgcctcgcgccgcccgccccggctctgactgaccgcg
ttactcccacaggtgagcgggcgggacggcccttctcctccgggctgtaattagcgcttggtttaatg
acggcttgtttcttttctgtggctgcgtgaaagccttgaggggctccgggagggccctttgtgcgggg
ggagcggctcggggggtgcgtgcgtgtgtgtgtgcgtggggagcgccgcgtgcggctccgcgctgccc
ggcggctgtgagcgctgcgggcgcggcgcggggctttgtgcgctccgcagtgtgcgcgaggggagcgc
ggccgggggcggtgccccgcggtgcggggggggctgcgaggggaacaaaggctgcgtgcggggtgtgt
gcgtgggggggtgagcagggggtgtgggcgcgtcggtcgggctgcaaccccccctgcacccccctccc
cgagttgctgagcacggcccggcttcgggtgcggggctccgtacggggcgtggcgcggggctcgccgt
gccgggcggggggtggcggcaggtgggggtgccgggcggggcggggccgcctcgggccggggagggct
cgggggaggggcgcggcggcccccggagcgccggcggctgtcgaggcgcggcgagccgcagccattgc
cttttatggtaatcgtgcgagagggcgcagggacttcctttgtcccaaatctgtgcggagccgaaatc
tgggaggcgccgccgcaccccctctagcgggcgcggggcgaagcggtgcggcgccggcaggaaggaaa
tgggcggggagggccttcgtgcgtcgccgcgccgccgtccccttctccctctccagcctcggggctgt
ccgcggggggacggctgccttcgggggggacggggcagggcggggttcggcttctggcgtgtgaccgg
cggctctagagcctctgctaaccatgttcatgccttcttctttttcctacagctcctgggcaacgtgc
tggttattgtgctgtctcatcattttggcaaagaacgcgttggacgcgtgtcgacgccaccgccacca
tggccctgtggatgagactgctgcctctgctggccctcctggccctgtggggccccgaccccgccgcc
gcttttgtgaaccagcacctgtgcggaagcgacctggtcgaggccctgtacctggtgtgcggcgagcg
gggcttcttctacacccctagaaccaagcgggaagccgaggatctgcaggtgggccaggtggagctgg
gcggcggaccaggcgccggctccctgcaacctctggctctggaaggatctagacagaagagaggcatc
gtggagcagtgttgtacaagcatctgcagcctgtatcagctggaaaactactgcaattaaacccagct
ttcttgtacaaagtgggaattcgagcatcttaccgccatttatacccatatttgttctgtttttcttg
atttgggtatacatttaaatgttaataaaacaaaatggtggggcaatcatttacatttttagggatat
gtaattactagttcaggtgtattgccacaagacaaacatgttaagaaactttcccgttatttacgctc
tgttcctgttaatcaacctctggattacaaaatttgtgaaagattgactgatattcttaactatgttg
ctccttttacgctgtgtggatatgctgctttatagcctctgtatctagctattgcttcccgtacggct
ttcgttttctcctccttgtataaatcctggttgctgtctcttttagaggagttgtggcccgttgtccg
tcaacgtggcgtggtgtgctctgtgtttgctgacgcaacccccactggctggggcattgccaccacct
gtcaactcctttctgggactttcgctttccccctcccgatcgccacggcagaactcatcgccgcctgc
cttgcccgctgctggacaggggctaggttgctgggcactgataattccgtggtgttgtcgaattccta
gagctcgctgatcagcctcgactgtgccttctagttgccagccatctgttgtttgcccctcccccgtg
ccttccttgaccctggaaggtgccactcccactgtcctttcctaataaaatgaggaaattgcatcgca
ttgtctgagtaggtgtcattctattctggggggtggggtggggcaggacagcaagggggaggattggg
aagagaatagcaggcatgctggggagggccgccgatcgtagttattaatagtaatcaattacggggtc
attagttcatagcccatatatggagttccgcgttacataacttacggtaaatggcccgcctggctgac
cgcccaacgacccccgcccattgacgtcaataatgacgtatgttcccatagtaacgccaatagggact
ttccattgacgtcaatgggtggagtatttacggtaaactgcccacttggcagtacatcaagtgtatca
tatgccaagtacgccccctattgacgtcaatgacggtaaatggcccgcctggcattatgcccagtaca
tgaccttatgggactttcctacttggcagtacatctacgtattagtcatcgctattaccatggtgatg
cggttttggcagtacatcaatgggcgtggatagcggtttgactcacggggatttccaagtctccaccc
cattgacgtcaatgggagtttgttttggcaccaaaatcaacgggactttccaaaatgtcgtaacaact
ccgccccattgacgcaaatgggcggtaggcgtgtacggtgggaggtctatataagcagagctggttta
gtgaaccgtcagatctcgcctggagacgccatccacgctgttttgacctccatagaagacaccgggac
cgatccagcctccgcggccgggaacggtgcattggaacgcggattccccgtgttaattaacaggtaag
tgtcttcctcctgtttccttcccctgctattctgctcaaccttcctatcagaaactgcagtatctgta
tttttgctagcagtaatactaacggttctttttttctcttcacaggccaccaagctaccggtccacca
tggactcccagcagccagatctgaagctactgtcttctatcgaacaagcatgcgatatttgccgactt
aaaaagctcaagtgctccaaagaaaaaccgaagtgcgccaagtgtctgaagaacaactgggagtgtcg
ctactctcccaaaaccaaaaggtctccgctgactagggcacatctgacagaagtggaatcaaggctag
aaagactggaacagctatttctactgatttttcctcgagaagaccttgacatgattttgaaaatggat
tctttacaggatataaaagcattgttagaattcccgggtgtcgaccagaaaaagttcaataaagtcag
agttgtgagagcactggatgctgttgctctcccacagccagtgggcgttccaaatgaaagccaagccc
taagccagagattcactttttcaccaggtcaagacatacagttgattccaccactgatcaacctgtta
atgagcattgaaccagatgtgatctatgcaggacatgacaacacaaaacctgacacctccagttcttt
gctgacaagtcttaatcaactaggcgagaggcaacttctttcagtagtcaagtggtctaaatcattgc
caggttttcgaaacttacatattgatgaccagataactctcattcagtattcttggatgagcttaatg
gtgtttggtctaggatggagatcctacaaacacgtcagtgggcagatgctgtattttgcacctgatct
aatactaaatgaacagcggatgaaagaatcatcattctattcattatgccttaccatgtggcagatcc
cacaggagtttgtcaagcttcaagttagccaagaagagttcctctgtatgaaagtattgttacttctt
aatacaattcctttggaagggctacgaagtcaaacccagtttgaggagatgaggtcaagctacattag
agagctcatcaaggcaattggtttgaggcaaaaaggagttgtgtcgagctcacagcgtttctatcaac
ttacaaaacttcttgataacttgcatgatcttgtcaaacaacttcatctgtactgcttgaatacattt
atccagtcccgggcactgagtgttgaatttccagaaatgatgtctgaagttattgctgggtcgacgcc
catggaattccagtacctgccagatacagacgatcgtcaccggattgaggagaaacgtaaaaggacat
atgagaccttcaagagcatcatgaagaagagtcctttcagcggacccaccgacccccggcctccacct
cgacgcattgctgtgccttcccgcagctcagcttctgtccccaagccagcaccccagccctatccctt
tacgtcatccctgagcaccatcaactatgatgagtttcccaccatggtgtttccttctgggcagatca
gccaggcctcggccttggccccggcccctccccaagtcctgccccaggctccagcccctgcccctgct
ccagccatggtatcagctctggcccaggccccagcccctgtcccagtcctagccccaggccctcctca
ggctgtggccccacctgcccccaagcccacccaggctggggaaggaacgctgtcagaggccctgctgc
agctgcagtttgatgatgaagacctgggggccttgcttggcaacagcacagacccagctgtgttcaca
gacctggcatccgtcgacaactccgagtttcagcagctgctgaaccagggcatacctgtggcccccca
cacaactgagcccatgctgatggagtaccctgaggctataactcgcctagtgacaggggcccagaggc
cccccgacccagctcctgctccactgggggccccggggctccccaatggcctcctttcaggagatgaa
gacttctcctccattgcggacatggacttctcagccctgctgagtcagatcagctcctaaggatcctc
cggactagaaaagccgaattctggggtggcatccctgtgacccctccccagtgcctctcctggccctg
gaagttgccactccagtgcccaccagccttgtcctaataaaattaagttgcatcattttgtctgacta
ggtgtccttctataatattatggggtggaggggggtggtatggagcaaggggcaagttgggaagacaa
cctgtagggcctgcggggtctattgggaaccaagctggagtgcagtggcacaatcttggctcactgca
atctccgcctcctgggttcaagcgattctcctgcctcagcctcccgagttgttgggattccaggcatg
catgaccaggctcagctaatttttgtttttttggtagagacggggtttcaccatattggccaggctgg
tctccaactcctaatctcaggtgatctacccaccttggcctcccaaattgctgggattacaggcgtga
accactgctcccttccctgtccttcaagtttgtacaaaaaagcaggctaacttgtttattgcagctta
taatggttacaaataaagcaatagcatcacaaatttcacaaataaagcatttttttcactgcattcta
gttgtggtttgtccaaactcatcaatgtatcttatttgtttgtttgagcgcgtttcgtcctatttggg
actcatcagctggatgtagtaggtggtcgtattcttcacctctccagatcgaaatctggtatgtttta
ccacgaatccaggacgcgcctttgtttgtttggactagaagcggccgcctattagttggcctctccca
tttcccgggcgaatgttctggccaggtcgcagatggtagggatagatccaggtgtggtcacgtgggtc
tggatcatgccgcttgtcagttgcagcagtgcatctctacatccggcaggagactggtcgtagtccag
gatgaacacgtgcataggcctcagtcttttagccagcacgtcgagggcccaggcaaacacgttgtaca
ggtcgccgttaggggccagcagctcaggggctctgaacagggtaaacagcgtatcgccgatgtggggt
ctagggccggcgttgctttgaggctcggcgccctgggggggcacggcggtcccgctcagctggcccca
gtcctctctccagcttccgccgccctgcaggtatctcacggtattggccagcaggccatacactctgc
ggatagcagccagcatggccagatccagtctctcgcctggccgctgccgcttggccagtctatcgatg
tgtctatcttctggcagggcgcccagcacgatgtttgtgccaggcagggtgggggggatcagggccac
gaaggccagcacggcctgaggtgtcatggagcccatcaggtatctagcagcggggtagcacagcagag
cggcgatggggtgccggtcgaagataagggtcagagctggaggaggggcgtggctgctaccggcttcg
ccgccaatatgaggagccagcacggcgtcggtgacggcgtagggcatgcccatggtgatctgggcgct
ggtcatcaccacggcggcgtcgccagcgctgatctcgccctgatccagtctgtgctgagtggtgtaga
tattggcgatggtttcagaggctcccaggacccgccagtaggtcatgggctcaggcacgtacacaatg
tcgtctctggagcccagggccacgagcagctgtgttgtggtggtcttgcccatgccgtgagggccgtc
gatgtaaacccgcagcagtgtaggcatggtggcagcctgcttttttgtacaaacttgagagtgaggac
gaacgcccccacccccttttatagccccccgctagcgtcttcggaggacagtactccggaattcgcgg
aggacagtactccgatcggaggacagtactccgctcgagtcggaggacagtactccgctcggaggaca
gtactccgcaacttttctatacaaagttgtctagaggtgtggaaagtccccaggctccccagcaggca
gaagtatgcaaagcatgcatctcaattagtcagcaaccaggtaccaggaacccctagtgatggagttg
gccactccctctctgcgcgctcgctcgctcactgaggccgggcgaccaaaggtcgcccgacgcccggg
ctttgcccgggcggcctcagtgagcgagcgagcgcgcag
SV40 DTS:
(SEQ ID NO: 6)
ggtgtggaaagtccccaggctccccagcaggcagaagtatgcaaagcatgcatctcaattagtcagca
acca
CAG promoter (CAGp):
(SEQ ID NO: 35)
ctagttattaatagtaatcaattacggggtcattagttcatagcccatatatggagttccgcgttaca
taacttacggtaaatggcccgcctggctgaccgcccaacgacccccgcccattgacgtcaataatgac
gtatgttcccatagtaacgccaatagggactttccattgacgtcaatgggtggagtatttacggtaaa
ctgcccacttggcagtacatcaagtgtatcatatgccaagtacgccccctattgacgtcaatgacggt
aaatggcccgcctggcattatgcccagtacatgaccttatgggactttcctacttggcagtacatcta
cgtattagtcatcgctattaccatggtcgaggtgagccccacgttctgcttcactctccccatctccc
ccccctccccacccccaattttgtatttatttattttttaattattttgtgcagcgatgggggcgggg
ggggggggggcgcgcgccaggcggggcggggcggggcgaggggcggggcggggcgaggcggagaggtg
cggcggcagccaatcagagcggcgcgctccgaaagtttccttttatggcgaggcggcggcggcggcgg
ccctataaaaagcgaagcgcgcggcgggcgggagtcgctgcgcgctgccttcgccccgtgccccgctc
cgccgccgcctcgcgccgcccgccccggctctgactgaccgcgttactcccacaggtgagcgggcggg
acggcccttctcctccgggctgtaattagcgcttggtttaatgacggcttgtttcttttctgtggctg
cgtgaaagccttgaggggctccgggagggccctttgtgcggggggagcggctcggggggtgcgtgcgt
gtgtgtgtgcgtggggagcgccgcgtgcggctccgcgctgcccggcggctgtgagcgctgcgggcgcg
gcgcggggctttgtgcgctccgcagtgtgcgcgaggggagcgcggccgggggcggtgccccgcggtgc
ggggggggctgcgaggggaacaaaggctgcgtgcggggtgtgtgcgtgggggggtgagcagggggtgt
gggcgcgtcggtcgggctgcaaccccccctgcacccccctccccgagttgctgagcacggcccggctt
cgggtgcggggctccgtacggggcgtggcgcggggctcgccgtgccgggcggggggtggcggcaggtg
ggggtgccgggcggggcggggccgcctcgggccggggagggctcgggggaggggcgcggcggcccccg
gagcgccggcggctgtcgaggcgcggcgagccgcagccattgccttttatggtaatcgtgcgagaggg
cgcagggacttcctttgtcccaaatctgtgcggagccgaaatctgggaggcgccgccgcaccccctct
agcgggcgcggggcgaagcggtgcggcgccggcaggaaggaaatgggcggggagggccttcgtgcgtc
gccgcgccgccgtccccttctccctctccagcctcggggctgtccgcggggggacggctgccttcggg
ggggacggggcagggcggggttcggcttctggcgtgtgaccggcggctctagagcctctgctaaccat
gttcatgccttcttctttttcctacagctcctgggcaacgtgctggttattgtgctgtctcatcattt
tggcaaagaacgc
Furin-cleavable human insulin (FC-hINS):
(SEQ ID NO: 36)
atggccctgtggatgagactgctgcctctgctggccctcctggccctgtggggccccgaccccgccgc
cgcttttgtgaaccagcacctgtgcggaagcgacctggtcgaggccctgtacctggtgtgcggcgagc
ggggcttcttctacacccctagaaccaagcgggaagccgaggatctgcaggtgggccaggtggagctg
ggcggcggaccaggcgccggctccctgcaacctctggctctggaaggatctagacagaagagaggcat
cgtggagcagtgttgtacaagcatctgcagcctgtatcagctggaaaactactgcaattaa
FC-hINS amino acid sequence:
(SEQ ID NO: 37)
MALWMRLLPLLALLALWGPDPAAAFVNQHLCGSDLVEALYLVCGERGFFYTPRTKREAEDLQVGQVEL
GGGPGAGSLQPLALEGSRQKRGIVEQCCTSICSLYQLENYCN
Optimized WPRE (oPRE):
(SEQ ID NO: 4)
gagcatcttaccgccatttatacccatatttgttctgtttttcttgatttgggtatacatttaaatgt
taataaaacaaaatggtggggcaatcatttacatttttagggatatgtaattactagttcaggtgtat
tgccacaagacaaacatgttaagaaactttcccgttatttacgctctgttcctgttaatcaacctctg
gattacaaaatttgtgaaagattgactgatattcttaactatgttgctccttttacgctgtgtggata
tgctgctttatagcctctgtatctagctattgcttcccgtacggctttcgttttctcctccttgtata
aatcctggttgctgtctcttttagaggagttgtggcccgttgtccgtcaacgtggcgtggtgtgctct
gtgtttgctgacgcaacccccactggctggggcattgccaccacctgtcaactcctttctgggacttt
cgctttccccctcccgatcgccacggcagaactcatcgccgcctgccttgcccgctgctggacagggg
ctaggttgctgggcactgataattccgtggtgttgtc
CMV promoter (CMVp):
(SEQ ID NO: 1)
tagttattaatagtaatcaattacggggtcattagttcatagcccatatatggagttccgcgttacat
aacttacggtaaatggcccgcctggctgaccgcccaacgacccccgcccattgacgtcaataatgacg
tatgttcccatagtaacgccaatagggactttccattgacgtcaatgggtggagtatttacggtaaac
tgcccacttggcagtacatcaagtgtatcatatgccaagtacgccccctattgacgtcaatgacggta
aatggcccgcctggcattatgcccagtacatgaccttatgggactttcctacttggcagtacatctac
gtattagtcatcgctattaccatggtgatgcggttttggcagtacatcaatgggcgtggatagcggtt
tgactcacggggatttccaagtctccaccccattgacgtcaatgggagtttgttttggcaccaaaatc
aacgggactttccaaaatgtcgtaacaactccgccccattgacgcaaatgggcggtaggcgtgtacgg
tgggaggtctatataagcagagctggtttagtgaaccgtcagatc
Synthetic intron IVS8:
(SEQ ID NO: 38)
gtaagtgtcttcctcctgtttccttcccctgctattctgctcaaccttcctatcagaaactgcagtat
ctgtatttttgctagcagtaatactaacggttctttttttctcttcacag
SWITCH Nucleic Acid Sequence:
(SEQ ID NO: 39)
atggactcccagcagccagatctgaagctactgtcttctatcgaacaagcatgcgatatttgccgact
taaaaagctcaagtgctccaaagaaaaaccgaagtgcgccaagtgtctgaagaacaactgggagtgtc
gctactctcccaaaaccaaaaggtctccgctgactagggcacatctgacagaagtggaatcaaggcta
gaaagactggaacagctatttctactgatttttcctcgagaagaccttgacatgattttgaaaatgga
ttctttacaggatataaaagcattgttagaattcccgggtgtcgaccagaaaaagttcaataaagtca
gagttgtgagagcactggatgctgttgctctcccacagccagtgggcgttccaaatgaaagccaagcc
ctaagccagagattcactttttcaccaggtcaagacatacagttgattccaccactgatcaacctgtt
aatgagcattgaaccagatgtgatctatgcaggacatgacaacacaaaacctgacacctccagttctt
tgctgacaagtcttaatcaactaggcgagaggcaacttctttcagtagtcaagtggtctaaatcattg
ccaggttttcgaaacttacatattgatgaccagataactctcattcagtattcttggatgagcttaat
ggtgtttggtctaggatggagatcctacaaacacgtcagtgggcagatgctgtattttgcacctgatc
taatactaaatgaacagcggatgaaagaatcatcattctattcattatgccttaccatgtggcagatc
ccacaggagtttgtcaagcttcaagttagccaagaagagttcctctgtatgaaagtattgttacttct
taatacaattcctttggaagggctacgaagtcaaacccagtttgaggagatgaggtcaagctacatta
gagagctcatcaaggcaattggtttgaggcaaaaaggagttgtgtcgagctcacagcgtttctatcaa
cttacaaaacttcttgataacttgcatgatcttgtcaaacaacttcatctgtactgcttgaatacatt
tatccagtcccgggcactgagtgttgaatttccagaaatgatgtctgaagttattgctgggtcgacgc
ccatggaattccagtacctgccagatacagacgatcgtcaccggattgaggagaaacgtaaaaggaca
tatgagaccttcaagagcatcatgaagaagagtcctttcagcggacccaccgacccccggcctccacc
tcgacgcattgctgtgccttcccgcagctcagcttctgtccccaagccagcaccccagccctatccct
ttacgtcatccctgagcaccatcaactatgatgagtttcccaccatggtgtttccttctgggcagatc
agccaggcctcggccttggccccggcccctccccaagtcctgccccaggctccagcccctgcccctgc
tccagccatggtatcagctctggcccaggccccagcccctgtcccagtcctagccccaggccctcctc
aggctgtggccccacctgcccccaagcccacccaggctggggaaggaacgctgtcagaggccctgctg
cagctgcagtttgatgatgaagacctgggggccttgcttggcaacagcacagacccagctgtgttcac
agacctggcatccgtcgacaactccgagtttcagcagctgctgaaccagggcatacctgtggcccccc
acacaactgagcccatgctgatggagtaccctgaggctataactcgcctagtgacaggggcccagagg
ccccccgacccagctcctgctccactgggggccccggggctccccaatggcctcctttcaggagatga
agacttctcctccattgcggacatggacttctcagccctgctgagtcagatcagctcctaa
SWITCH Amino Acid Sequence:
(SEQ ID NO: 40)
MDSQQPDLKLLSSIEQACDICRLKKLKCSKEKPKCAKCLKNNWECRYSPKTKRSPLTRAHLTEVESRL
ERLEQLFLLIFPREDLDMILKMDSLQDIKALLEFPGVDQKKFNKVRVVRALDAVALPQPVGVPNESQA
LSQRFTFSPGQDIQLIPPLINLLMSIEPDVIYAGHDNTKPDTSSSLLTSLNQLGERQLLSVVKWSKSL
PGFRNLHIDDQITLIQYSWMSLMVFGLGWRSYKHVSGQMLYFAPDLILNEQRMKESSFYSLCLIMWQI
PQEFVKLQVSQEEFLCMKVLLLLNTIPLEGLRSQTQFEEMRSSYIRELIKAIGLRQKGVVSSSQRFYQ
LTKLLDNLHDLVKQLHLYCLNTFIQSRALSVEFPEMMSEVIAGSTPMEFQYLPDTDDRHRIEEKRKRT
YETFKSIMKKSPFSGPTDPRPPPRRIAVPSRSSASVPKPAPQPYPFTSSLSTINYDEFPTMVFPSGQI
SQASALAPAPPQVLPQAPAPAPAPAMVSALAQAPAPVPVLAPGPPQAVAPPAPKPTQAGEGTLSEALL
QLQFDDEDLGALLGNSTDPAVFTDLASVDNSEFQQLLNQGIPVAPHTTEPMLMEYPEAITRLVTGAQR
PPDPAPAPLGAPGLPNGLLSGDEDFSSIADMDFSALLSQISS
hGH pA:
(SEQ ID NO: 41)
gggtggcatccctgtgacccctccccagtgcctctcctggccctggaagttgccactccagtgcccac
cagccttgtcctaataaaattaagttgcatcattttgtctgactaggtgtccttctataatattatgg
ggtggaggggggtggtatggagcaaggggcaagttgggaagacaacctgtagggcctgcggggtctat
tgggaaccaagctggagtgcagtggcacaatcttggctcactgcaatctccgcctcctgggttcaagc
gattctcctgcctcagcctcccgagttgttgggattccaggcatgcatgaccaggctcagctaatttt
tgtttttttggtagagacggggtttcaccatattggccaggctggtctccaactcctaatctcaggtg
atctacccaccttggcctcccaaattgctgggattacaggcgtgaaccactgctcccttccctgtcct
t
GAL4 UAS:
(SEQ ID NO: 42)
Cggagtactgtcctccgagcggagtactgtcctccgactcgagcggagtactgtcctccgatcggagt
actgtcctccgcgaattccggagtactgtcctccg
Minimal Ad Promoter (ADp):
(SEQ ID NO: 43)
Ggggggctataaaagggggtgggggcgttcgtcctcactct

### Example 3

This example outlines a DNA minicircle construct, delivered within a liposome that contains an inflammation responsive promoter, post-translational regulatory element, gene of interest (interleukin-1 receptor antagonist), nuclear targeting element, and a genetic regulatory element that enables the reduction of gene of interest expression following administration of a rapamycin.

### Genetic Construct Map (Excluding Plasmid Backbone):

Full-length Construct Sequence (excluding plasmid backbone):
(SEQ ID NO: 44)
ctgcgcgctcgctcgctcactgaggccgcccgggcaaagcccgggcgtcgggcgacctttggtcgccc
ggcctcagtgagcgagcgagcgcgcagagagggagtggccaactccatcactaggggttccttctaga
tggggactttccgctggggactttccgctggggactttccgctggggactttccgctggggactttcc
gccaactttgtatagaaaagttgGGGAATTTCCGGGGACTTTCCGGGAATTTCCGGGGACTTTCCGGG
AATTTCCAGATCTGGCCTCGGCGGCCAAGCTTAGACACTAGAGGGTATATAATGGAAGCTCGACTTCC
AGcaagtttgtacaaaaaagcaggctgtaagggtttaagggatggttggttggtggggtattaatgtt
taattacctggagcacctgcctgaaatcactttttttcaggttggacgcgtgtcgacgccaccgccac
catggaaatctgtagaggcctgaggtctcatctgattaccctgctgctgttcctgttccacagcgaaa
ccatctgcagacctagcggcagaaagagcagcaagatgcaagctttcagaatctgggatgtgaaccag
aagaccttctacctgcggaacaaccagctggtggccggctacctgcagggccctaatgtgaatctgga
agagaaaatcgacgtcgtgcccatcgagcctcacgccctgtttctgggcatccacggcggcaagatgt
gcctgagctgcgtgaagtctggagatgagacaagactgcagctcgaggccgtgaacatcacagacctg
tccgagaacagaaaacaggacaagcggttcgccttcatccggagcgacagcggaccaaccaccagctt
cgagtctgccgcttgtcccggctggttcctgtgcacagccatggaagccgaccagcctgtgtccctga
ccaacatgcctgatgaaggcgtgatggtgaccaagttctactttcaggaggacgagtaaacccagctt
tcttgtacaaagtgggaattcgagcatcttaccgccatttatacccatatttgttctgtttttcttga
tttgggtatacatttaaatgttaataaaacaaaatggtggggcaatcatttacatttttagggatatg
taattactagttcaggtgtattgccacaagacaaacatgttaagaaactttcccgttatttacgctct
gttcctgttaatcaacctctggattacaaaatttgtgaaagattgactgatattcttaactatgttgc
tccttttacgctgtgtggatatgctgctttatagcctctgtatctagctattgcttcccgtacggctt
tcgttttctcctccttgtataaatcctggttgctgtctcttttagaggagttgtggcccgttgtccgt
caacgtggcgtggtgtgctctgtgtttgctgacgcaacccccactggctggggcattgccaccacctg
tcaactcctttctgggactttcgctttccccctcccgatcgccacggcagaactcatcgccgcctgcc
ttgcccgctgctggacaggggctaggttgctgggcactgataattccgtggtgttgtcgaattcctag
agctcgctgatcagcctcgactgtgccttctagttgccagccatctgttgtttgcccctcccccgtgc
cttccttgaccctggaaggtgccactcccactgtcctttcctaataaaatgaggaaattgcatcgcat
tgtctgagtaggtgtcattctattctggggggtggggtggggcaggacagcaagggggaggattggga
agagaatagcaggcatgctggggagggccgccgatcgaacttgtttattgcagcttataatggttaca
aataaagcaatagcatcacaaatttcacaaataaagcatttttttcactgcattctagttgtggtttg
tccaaactcatcaatgtatcttatttgtttgtttgagcgcgtttcgtcctatttgggactcatcagct
ggatgtagtaggtggtcgtattcttcacctctccagatcgaaatctggtatgttttaccacgaatcca
ggacgcgcctttgtttgtttggactagaagcggccgcctattagctggcgctggtcttgaaaaacagc
ttctttctcaagaagttgaagcagccaggcatctgcttgtagatgcccttaacggacacggcattagc
cacccgcagcagcagagattgaaggtcctcgctgtgggcccactgctcaaagatatcgtccagtgtct
ccacgtaccaggagccgctcttagggtcccgccagctcacgaatcctggaaaggtgctatatgacacg
aagatatcgctaggtgtaggcaggctggagatggcatccagctggtcaaaagttctcaggccctcctg
aaaaggggtggcgtctggctcaggattagagcctggagactcgtcctcggggcttgtgctggccacct
cgaagccgtggtccttctgttcgccgccacaagcctggatgaagaacagcttgggcttgccgcccagg
ctggggcaggatgtgccgttgaagatgttcacgatcttttccacggacacagggcaaccgtcggtgcc
gtacactgctccagggaactgcaggtggctagcctgacagccatggctcaggatgaccaccacgcagc
agtccagggcgccgtggtcctgctgggccagttcgagcagggccagcaccatcttcttggcggtgaga
tctcctttcacctcgaccatgaagtgcagagagctgaatcttctccgcagcttctcgcagtcgatgtt
gctgccggttctggtgcgcagtccgctctctctgcagaagttcacgttgttgatgatcaggcagtggc
cacaaggctccatgctcagaatgtaagccagatcggcgttgcctctgaggctttccagggcgcccacg
tcgccgaagccgtccacgccggaaccgccgccgccgctgccaccgcctccagagccgccgcctccgct
ctccagcttcagcagctccacatcaaagaccagggtggcgtggggggggatgatgccagggtgtccgg
tagcgccgtaggcgtagtcaggagaaattgtcagtttggcccgctggcccacgctcatttgggccacg
ccttcctcccagcctctgatcacctcctgtttgcccagcatgaacttgaaaggcttgttccggtctct
gctgctgtcgaatttctttccatcttccagcattccggtgtagtgcaccacacaggtctgtcctcttt
tggggaatgttctgccatcgccaggagagatggtttccacctgaactccttccaggcttcctcctccg
ctgtattccagcttgctgatccgtctgaacacgtggtagtacaggtcccaggcttgcagcaggtcctt
cacattcccgcttttcatgtacttccggcaccactcctgggcttccatcagatcgcggccataggcct
ggttgaagcttgtttccttcagggtctgagggcctctttccatcatggcgtgcaggggttccaggacc
tcgaacatgcccttcacgttccgctcgccgaagtacagtctactagcctcttccaggccctcgtgcca
catctcatgccacaggattctagaggccatggtggcagcctgcttttttgtacaaacttggatctgac
ggttcactaaaccagctctgcttatatagacctcccaccgtacacgcctaccgcccatttgcgtcaat
ggggcggagttgttacgacattttggaaagtcccgttgattttggtgccaaaacaaactcccattgac
gtcaatggggtggagacttggaaatccccgtgagtcaaaccgctatccacgcccattgatgtactgcc
aaaaccgcatcaccatggtaatagcgatgactaatacgtagatgtactgccaagtaggaaagtcccat
aaggtcatgtactgggcataatgccaggcgggccatttaccgtcattgacgtcaatagggggcgtact
tggcatatgatacacttgatgtactgccaagtgggcagtttaccgtaaatactccacccattgacgtc
aatggaaagtccctattggcgttactatgggaacatacgtcattattgacgtcaatgggcgggggtcg
ttgggcggtcagccaggcgggccatttaccgtaagttatgtaacgcggaactccatatatgggctatg
aactaatgaccccgtaattgattactattaataactacaacttttctatacaaagttgtctagatggg
gactttccgctggggactttccgctggggactttccgctggggactttccgctggggactttccgcgg
taccaggaacccctagtgatggagttggccactccctctctgcgcgctcgctcgctcactgaggccgg
gcgaccaaaggtcgcccgacgcccgggctttgcccgggcggcctcagtgagcgagcgagcgcgcag
5xNFkB DTS:
(SEQ ID NO: 45)
tggggactttccgctggggactttccgctggggactttccgctggggactttccgctggggactttcc
gc
NFkB response element (RE):
(SEQ ID NO: 46)
gggaatttccggggactttccgggaatttccggggactttccgggaatttcc
Minimal promoter (minP):
(SEQ ID NO: 47)
Tagagggtatataatggaagctcgacttccag
MVM intron:
(SEQ ID NO: 48)
gtaagggtttaagggatggttggttggtggggtattaatgtttaattacctggagcacctgcctgaaa
tcactttttttcag
Interleukin-1 Receptor Antagonist (IL-1 RA) Sequence:
(SEQ ID NO: 49)
atggaaatctgtagaggcctgaggtctcatctgattaccctgctgctgttcctgttccac
agcgaaaccatctgcagacctagcggcagaaagagcagcaagatgcaagctttcagaatc
tgggatgtgaaccagaagaccttctacctgcggaacaaccagctggtggccggctacctg
cagggccctaatgtgaatctggaagagaaaatcgacgtcgtgcccatcgagcctcacgcc
ctgtttctgggcatccacggcggcaagatgtgcctgagctgcgtgaagtctggagatgag
acaagactgcagctcgaggccgtgaacatcacagacctgtccgagaacagaaaacaggac
aagcggttcgccttcatccggagcgacagcggaccaaccaccagcttcgagtctgccgct
tgtcccggctggttcctgtgcacagccatggaagccgaccagcctgtgtccctgaccaac
atgcctgatgaaggcgtgatggtgaccaagttctactttcaggaggacgagtaa
Interleukin-1 receptor antagonist (IL-1 RA) Amino Acid Sequence:
(SEQ ID NO: 50)
MEICRGLRSHLITLLLFLFHSETICRPSGRKSSKMQAFRIWDVNQKTFYLRNNQLVAGYLQGPNVNLE
EKIDVVPIEPHALFLGIHGGKMCLSCVKSGDETRLQLEAVNITDLSENRKQDKRFAFIRSDSGPTTSF
ESAACPGWFLCTAMEADQPVSLINMPDEGVMVTKFYFQEDE
rapaCas9 Sequence:
(SEQ ID NO: 51)
atggcctctagaatcctgtggcatgagatgtggcacgagggcctggaagaggctagtagactgtactt
cggcgagcggaacgtgaagggcatgttcgaggtcctggaacccctgcacgccatgatggaaagaggcc
ctcagaccctgaaggaaacaagcttcaaccaggcctatggccgcgatctgatggaagcccaggagtgg
tgccggaagtacatgaaaagcgggaatgtgaaggacctgctgcaagcctgggacctgtactaccacgt
gttcagacggatcagcaagctggaatacagcggaggaggaagcctggaaggagttcaggtggaaacca
tctctcctggcgatggcagaacattccccaaaagaggacagacctgtgtggtgcactacaccggaatg
ctggaagatggaaagaaattcgacagcagcagagaccggaacaagcctttcaagttcatgctgggcaa
acaggaggtgatcagaggctgggaggaaggcgtggcccaaatgagcgtgggccagcgggccaaactga
caatttctcctgactacgcctacggcgctaccggacaccctggcatcatccccccccacgccaccctg
gtctttgatgtggagctgctgaagctggagagcggaggcggcggctctggaggcggtggcagcggcgg
cggcggttccggcgtggacggcttcggcgacgtgggcgccctggaaagcctcagaggcaacgccgatc
tggcttacattctgagcatggagccttgtggccactgcctgatcatcaacaacgtgaacttctgcaga
gagagcggactgcgcaccagaaccggcagcaacatcgactgcgagaagctgcggagaagattcagctc
tctgcacttcatggtcgaggtgaaaggagatctcaccgccaagaagatggtgctggccctgctcgaac
tggcccagcaggaccacggcgccctggactgctgcgtggtggtcatcctgagccatggctgtcaggct
agccacctgcagttccctggagcagtgtacggcaccgacggttgccctgtgtccgtggaaaagatcgt
gaacatcttcaacggcacatcctgccccagcctgggcggcaagcccaagctgttcttcatccaggctt
gtggcggcgaacagaaggaccacggcttcgaggtggccagcacaagccccgaggacgagtctccaggc
tctaatcctgagccagacgccaccccttttcaggagggcctgagaacttttgaccagctggatgccat
ctccagcctgcctacacctagcgatatcttcgtgtcatatagcacctttccaggattcgtgagctggc
gggaccctaagagcggctcctggtacgtggagacactggacgatatctttgagcagtgggcccacagc
gaggaccttcaatctctgctgctgcgggtggctaatgccgtgtccgttaagggcatctacaagcagat
gcctggctgcttcaacttcttgagaaagaagctgtttttcaagaccagcgccagctaa
rapaCas9 Amino Acid Sequence:
(SEQ ID NO: 13)
MASRILWHEMWHEGLEEASRLYFGERNVKGMFEVLEPLHAMMERGPQTLKETSFNQAYGRDLMEAQEW
CRKYMKSGNVKDLLQAWDLYYHVFRRISKLEYSGGGSLEGVQVETISPGDGRTFPKRGQTCVVHYTGM
LEDGKKFDSSRDRNKPFKFMLGKQEVIRGWEEGVAQMSVGQRAKLTISPDYAYGATGHPGIIPPHATL
VFDVELLKLESGGGGSGGGGSGGGGSGVDGFGDVGALESLRGNADLAYILSMEPCGHCLIINNVNFCR
ESGLRTRTGSNIDCEKLRRRFSSLHFMVEVKGDLTAKKMVLALLELAQQDHGALDCCVVVILSHGCQA
SHLQFPGAVYGTDGCPVSVEKIVNIFNGTSCPSLGGKPKLFFIQACGGEQKDHGFEVASTSPEDESPG
SNPEPDATPFQEGLRTFDQLDAISSLPTPSDIFVSYSTFPGFVSWRDPKSGSWYVETLDDIFEQWAHS
EDLQSLLLRVANAVSVKGIYKQMPGCFNFLRKKLFFKTSAS

### Example 4

This example outlines a double-stranded DNA construct, delivered within a ligand-targeted polymeric particle that contains a constitutive promoter, post-translational regulatory element, gene of interest (hBDNF), nuclear targeting element, and a genetic regulatory element that enables the reduction of gene of interest expression following administration of a doxycycline.

### Genetic Construct Map:

Full-length Construct Sequence (excluding plasmid backbone):
(SEQ ID NO: 52)
ctgcgcgctcgctcgctcactgaggccgcccgggcaaagcccgggcgtcgggcgacctttggtcgccc
ggcctcagtgagcgagcgagcgcgcagagagggagtggccaactccatcactaggggttcctatcgat
caactttgtatagaaaagttgggtacattttgttctagaacaaaatgtaccggtacattttgttctgg
tacattttgttctcaactttgtatagaaaagttgggtaccgagctcgactttcacttttctctatcac
tgatagggagtggtaaactcgactttcacttttctctatcactgatagggagtggtaaactcgacttt
cacttttctctatcactgatagggagtggtaaactcgactttcacttttctctatcactgatagggag
tggtaaactcgactttcacttttctctatcactgatagggagtggtaaactcgactttcacttttctc
tatcactgatagggagtggtaaactcgactttcacttttctctatcactgatagggagtggtaaactc
gacctatataagcagagctcgtttagtgaaccgtcagatcgcctggagacgccatccacgctgttttg
acctccatagaagacaccgggaccgatccagcctccgcggccccgaattgcaagtttgtacaaaaaag
caggctgccaccatggcctctagaatcctgtggcatgagatgtggcacgagggcctggaagaggctag
tagactgtacttcggcgagcggaacgtgaagggcatgttcgaggtcctggaacccctgcacgccatga
tggaaagaggccctcagaccctgaaggaaacaagcttcaaccaggcctatggccgcgatctgatggaa
gcccaggagtggtgccggaagtacatgaaaagcgggaatgtgaaggacctgctgcaagcctgggacct
gtactaccacgtgttcagacggatcagcaagctggaatacagcggaggaggaagcctggaaggagttc
aggtggaaaccatctctcctggcgatggcagaacattccccaaaagaggacagacctgtgtggtgcac
tacaccggcatgctggaagatggaaagaaattcgacagcagcagagaccggaacaagcctttcaagtt
catgctgggcaaacaggaggtgatcagaggctgggaggaaggcgtggcccaaatgagcgtgggccagc
gggccaaactgacaatttctcctgactacgcctacggcgctaccggacaccctggcatcatccccccc
cacgccaccctggtctttgatgtggagctgctgaagctggagagcggaggcggcggctctggaggcgg
tggcagcggcggcggcggttccggcgtggacggcttcggcgacgtgggcgccctggaaagcctcagag
gcaacgccgatctggcttacattctgagcatggagccttgtggccactgcctgatcatcaacaacgtg
aacttctgcagagagagcggactgcgcaccagaaccggcagcaacatcgactgcgagaagctgcggag
aagattcagctctctgcacttcatggtcgaggtgaaaggagatctcaccgccaagaagatggtgctgg
ccctgctcgaactggcccagcaggaccacggcgccctggactgctgcgtggtggtcatcctgagccat
ggctgtcaggctagccacctgcagttccctggagcagtgtacggcaccgacggttgccctgtgtccgt
ggaaaagatcgtgaacatcttcaacggcacatcctgccccagcctgggcggcaagcccaagctgttct
tcatccaggcttgtggcggcgaacagaaggaccacggcttcgaggtggccagcacaagccccgaggac
gagtctccaggctctaatcctgagccagacgccaccccttttcaggagggcctgagaacttttgacca
gctggatgccatctccagcctgcctacacctagcgatatcttcgtgtcatatagcacctttccaggat
tcgtgagctggcgggaccctaagagcggctcctggtacgtggagacactggacgatatctttgagcag
tgggcccacagcgaggaccttcaatctctgctgctgcgggtggctaatgccgtgtccgttaagggcat
ctacaagcagatgcctggctgcttcaacttcttgagaaagaagctgtttttcaagaccagcgccagct
aaacccagctttcttgtacaaagtggtgatggccggccgcttcgagcagacatgataagatacattga
tgagtttggacaaaccacaactagaatgcagtgaaaaaaatgctttatttgtgaaatttgtgatgcta
ttgctttatttgtaaccattataagctgcaataaacaagttaacaacaacaattgcattcattttatg
tttcaggttcagggggaggtgtgggaggttttttaaagcaagtaaaacctctacaaatgtggtaatcg
atccatagagcccaccgcatccccagcatgcctgctattgtcttcccaatcctcccccttgctgtcct
gccccaccccaccccccagaatagaatgacacctactcagacaatgcgatgcaatttcctcattttat
taggaaaggacagtgggagtggcaccttccagggtcaaggaaggcacgggggaggggcaaacaacaga
tggctggcaactagaaggcacagttacccggggagcatgtcaaggtcaaaatcgtcaagagcgtcagc
aggcagcatatcaaggtcaaagtcgtcaagggcatcggctgggagcatgtctaagtcaaaatcgtcaa
gggcgtcggccggcccgccgctttcgcactttagctgtttctccaggccacatatgattagttccagg
ccgaaaaggaaggcaggttcggctccctgccggtcgaacagctcaattgcttgtctcagaagtggggg
catagaatcggtggtaggtgtctctctttcctcttttgctacttgatgctcctgttcctccaatacgc
agcccagtgtaaagtggcccacggcggacagagcgtacagtgcgttctccagggagaagccttgctga
cacaggaacgcgagctgattttccagggtttcgtactgtttctctgttgggcgggtgccgagatgcac
tttagccccgtcgcgatgtgagaggagagcacagcggaatgacttggcgttgttccgcagaaagtctt
gccatgactcgccttccagggggcagaagtgggtatgatgcctgtccagcatctcgattggcagggca
tcgagcagggcccgcttgttcttcacgtgccagtacagggtaggctgctcaactcccagcttttgagc
gagtttccttgtcgtcaggccttcgataccgactccattgagtaattccagagcgccgtttatgactt
tgctcttgtccagtctagacatggggccgggattttcctccacgtccccgcatgttagaagacttccc
ctgccctctccggagccccagggatcctctccttgctgcaacagggagatcacctttggtttggtaaa
caggaatcctgccatggaggccaggttgctgtaattctccagcatcacctcacggtacaggcttctct
gagacagatccagcttcttccactcgtcccgagtaaagagcacagccacatcttcaaatgtcactgac
actgctagctttctcttcttttttggtttattaccatcctcaatgggtgtatgctttttttcatctaa
ctgtgcagaaaatccaaatattaatgatttcagcccgaaatggaaggccatttcagcagaggttctcg
cctgaatattaaatgcctcttgtaataatggtggtgcagcgtctatcacatgattattttctatctgg
ttgtttgcttgctcctctaatactgcacccaacgtaaaatggctgatagattgcagaatgaaaagagc
ctcctcgaccgaaaaccctgcatcgcatagacagcgtagttgcgcctctgcttgttcaaactgggggg
gcgtaggagaggtccctatatgcaatcgggctccatcacgatggaccagtaatgctttacggaaactc
agagcattttcctggagaaactgctgccaactctcagtcggtaacggtgctgaacgggtgtgatgctt
cgccagtattgcctctgaaagcatgttcataagagtctgcttgttgcgcacgtgccaatacaatgtag
gctgctctacacctagcttctgggcgagtttacgggttgttaaaccttcgattccgacctcattaagc
agctctaatgcgctgttaatcactttacttttatctaatctagacatgaattcccaacctgaaaaaaa
gtgatttcaggcaggtgctccaggtaattaaacattaataccccaccaaccaaccatcccttaaaccc
ttacctcttgctcagctaattacagcccggaggagaagggccgtcccgcccgctcacctgtgggagta
acgcggtcagtcagagccggggcgggcggcgcgaggcggcggcggagcggggcacggggcgaaggcag
cgcgcagcgactcccgcccgccgcgcgcttcgctttttatagggccgccgccgccgccgcctcgccat
aaaaggaaactttcggagcgcgccgctctgattggctgccgccgcacctctccgcctcgccccgcccc
gcccctcgccccgccccgccccgcctggcgcgcgccccccccccccccccgcccccatcgctgcacaa
aataattaaaaaataaataaatacaaaattgggggtggggaggggggggagatggggagagtgaagca
gaacgtggggctcacctcgaccatggtaatagcgatgactaatacgtagatgtactgccaagtaggaa
agtcccataaggtcatgtactgggcacaatgccaggcgggccatttaccgtcattgacgtcaataggg
ggcgtacttggcatatgatacacttgatgtactgccaagtgggcagtttaccgtaaatactccaccca
ttgacgtcaatggaaagtccctattggcgttactattgacgtcaatgggcgggggtcgttgggcggtc
agccaggcgggccatttaccgtaagttatgtaacggggccgcgggccgccgatcgaacttgtttattg
cagcttataatggttacaaataaagcaatagcatcacaaatttcacaaataaagcatttttttcactg
cattctagttgtggtttgtccaaactcatcaatgtatcttatttgtttgtttgagcgcgtttcgtcct
atttgggactcatcagctggatgtagtaggtggtcgtattcttcacctctccagatcgaaatctggta
tgttttaccacgaatccaggacgcgcctttgtttgtttggactagaagcggccgcctaccgatgcggg
gaggcggcccaaagggagatccgactcgtctgagggcgaaggcgaagacgcggaagaggccgcagagc
cggcagcaggccgcgggaaggaaggtccgctggattgagggccgaagggacgtagcagaaggacgtcc
cgcgcagaatccaggtggcaacacaggcgagcagccatggaaaggacgtcagcttccccgacaacacc
acggaattgtcagtgcccaacagccgagcccctgtccagcagcgggcaaggcaggcggcgatgagttc
cgccgtggcaatagggagggggaaagcgaaagtcccggaaaggagctgacaggtggtggcaatgcccc
aaccagtgggggttgcgtcagcaaacacagtgcacaccacgccacgttgcctgacaacgggccacaac
tcctcataaagagacagcaaccaggatttatacaaggaggagaaaatgaaagccatacgggaagcaat
agcatgatacaaaggcattaaagcagcgtatccacatagcgtaaaaggagcaacatagttaagaatac
cagtcaatctttcacaaattttgtaatccagaggttgattatcggaattcccactttgtacaagaaag
ctgggtctattatcttcctctcttaatggtcagggtacacacgcagctggtgtcgattctgatgaacc
gccagccgatccgcttcttgctgtccattgtcagggcccgcacataagattgggtggttctgcactgg
ctgttccagtggcgcttgtcgatgcctctacagccttcctttgtgtagcccatggggttgcattttgt
ctcgtagaagtactgcttcagctggcccttggacacaggcaccttttccagcacggtcactgttccgc
cgctcatgtccacggcggttttcttatcagcggctgtgacccactcgctgatgctgtcgcacacgctc
agctcgccccttctggcaggatcagagtgggctttcatgcagccaaagtaggagatcaccatggtcag
gaacaggatggtcatggtggcagcctgcttttttgtacaaacttgtcacgacacctgaaatggaagaa
aaaaactttgaaccactgtctgaggcttgagaatgaaccaagatccaaactcaaaaagggcaaattcc
aaggagaattacatcaagtgccaagctggcctaacttcagtctccacccactcagtgtggggaaactc
catcgcataaaacccctccccccaacctaaagacgacgtactccaaaagctcgagaactaatcgaggt
gcctggacggcgcccggtactccgtggagtcacatgaagcgacggctgaggacggaaaggcccttttc
ctttgtgtgggtgactcacccgcccgctctcccgagcgccgcgtcctccattttgagctccctgcagc
agggccgggaagcggccatctttccgctcacgcaactggtgccgaccgggccagccttgccgcccagg
gcggggcgatacacggcggcgcgaggccaggcaccagagcaggccggccagcttgagactacccccgt
ccgattctcggtggccgcgctcgcaggccccgcctcgccgaacatgtgcgctgggacgcacgggcccc
gtcgccgcccgcggccccaaaaaccgaaataccagtgtgcagatcttggcccgcatttacaagactat
cttgccagaaaaaaagcgtcgcagcaggtcatcaaaaattttaaatggctagagacttatcgaaagca
gcgagacaggcgcgaaggtgccaccagattcgcacgcggcggccccagcgcccaggccaggcctcaac
tcaagcacgaggcgaaggggctccttaagcgcaaggcctcgaactctcccacccacttccaacccgaa
gctcgggatcaagaatcacgtactgcagccaggtggaagtaattcaaggcacgcaagggccataaccc
gtaaagaggccaggcccgcgggaaccacacacggcacttacctgtgttctggcggcaaacccgttgcg
aaaaagaacgttcacggcgactactgcacttatatacggttctcccccaccctcgggaaaaaggcgga
gccagtacacgacatcactttcccagtttaccccgcgccaccttctctaggcaccggttcaattgccg
acccctccccccaacttctcggggactgtgggcgatgtgcgctctgcccactgacgggcaccggagcc
caacttttctatacaaagttgtctagaggtacattttgttctagaacaaaatgtaccggtacattttg
ttctggtacattttgttctagatctaggaacccctagtgatggagttggccactccctctctgcgcgc
tcgctcgctcactgaggccgggcgaccaaaggtcgcccgacgcccgggctttgcccgggcggcctcag
tgagcgagcgagcgcgcag
WPRE:
(SEQ ID NO: 5)
Cgataatcaacctctggattacaaaatttgtgaaagattgactggtattcttaactatgttgctcctt
ttacgctatgtggatacgctgctttaatgcctttgtatcatgctattgcttcccgtatggctttcatt
ttctcctccttgtataaatcctggttgctgtctctttatgaggagttgtggcccgttgtcaggcaacg
tggcgtggtgtgcactgtgtttgctgacgcaacccccactggttggggcattgccaccacctgtcagc
tcctttccgggactttcgctttccccctccctattgccacggcggaactcatcgccgcctgccttgcc
cgctgctggacaggggctcggctgttgggcactgacaattccgtggtgttgtcggggaagctgacgtc
ctttccatggctgctcgcctgtgttgccacctggattctgcgcgggacgtccttctgctacgtccctt
cggccctcaatccagcggaccttccttcccgcggcctgctgccggctctgcggcctcttccgcgtctt
cgccttcgccctcagacgagtcggatctccctttgggccgcctccccgcatcgg
Mature BDNF (mBDNF)Sequence:
(SEQ ID NO: 53)
Atgaccatcctgttcctgaccatggtgatctcctactttggctgcatgaaagcccactctgatcctgc
cagaaggggcgagctgagcgtgtgcgacagcatcagcgagtgggtcacagccgctgataagaaaaccg
ccgtggacatgagcggcggaacagtgaccgtgctggaaaaggtgcctgtgtccaagggccagctgaag
cagtacttctacgagacaaaatgcaaccccatgggctacacaaaggaaggctgtagaggcatcgacaa
gcgccactggaacagccagtgcagaaccacccaatcttatgtgcgggccctgacaatggacagcaaga
agcggatcggctggcggttcatcagaatcgacaccagctgcgtgtgtaccctgaccattaagagagga
agataatag
mBDNF Amino Acid Sequence:
(SEQ ID NO: 54)
MTILFLTMVISYFGCMKAHSDPARRGELSVCDSISEWVTAADKKTAVDMSGGTVTVLEKVPVSKGQLK
QYFYETKCNPMGYTKEGCRGIDKRHWNSQCRTTQSYVRALTMDSKKRIGWRFIRIDTSCVCTLTIKRG
R
GRE DTS:
(SEQ ID NO: 55)
Ggtacattttgttctagaacaaaatgtaccggtacattttgttctggtacattttgttct

### Example 5

A therapeutic gene therapy formulation containing a candidate gene intended for the replacement of a human protein was produced by combining the following lipid components per ml of mixture.

TABLE 8

| Lipid | Concen- | | | | Required |
|---|---|---|---|---|---|
| Mixture | tration, | | Molar | Amount, | Volume, |
| Component | mg/ml | MW | ratio | mg | ul |
| SM-102 | 100 | 710.2 | 50 | 3.55 | 36 |
| 1,2-DSPC | 25 | 790.2 | 10 | 0.79 | 32 |
| Cholesterol | 5 | 386.7 | 38.5 | 1.48 | 296 |
| DMG- | 1 | 2,526 | 1.5 | 0.38 | 378 |
| PEG(2,000) | | | | | |
| EtOH | | | | | 258 |
| Total | | | | | 1000 |
| Volume | | | | | |
| Total | | | | 6.2 | |
| Amount | | | | | |

The formulations were combined with genetic constructs at N/P ratios of 4 to 10 using impingement jet mixing and evaluated for encapsulation efficiency.

TABLE 9

| | | | | Adjusted |
|---|---|---|---|---|
| | N/P | Concentration, | | Conc., |
| LNP ID | ratio | ug/ml | Encapsulation % | ug/ml |
| LNP102- | 4 | 64.75 | 39.9 | 25.84 |
| npRB2.6v7 | | | | |
| LNP102- | 5 | 54.19 | 57.3 | 31.05 |
| npRB2.6v7 | | | | |
| LNP102- | 6 | 57.49 | 63.1 | 36.29 |
| npRB2.6v7 | | | | |
| LNP102- | 7 | 52.00 | 81.1 | 42.19 |
| npRB2.6v7 | | | | |
| LNP102- | 10 | 31.78 | 91.6 | 29.10 |
| npRB2.6v7 | | | | |

TABLE 10

| | | | | Adjusted |
|---|---|---|---|---|
| | N/P | Concentration, | | Conc., |
| LNP ID | ratio | ug/ml | Encapsulation % | ug/ml |
| LNP102- | 7 | 108.29 | 94.54 | 102.38 |
| npRB2.6v3 | | | | |
| LNP102- | 8 | 96.66 | 94.65 | 91.49 |
| npRB2.6v3 | | | | |
| LNP102- | 9 | 96.68 | 96.35 | 93.15 |
| npRB2.6v3 | | | | |
| LNP102- | 10 | 77.22 | 95.30 | 73.59 |
| npRB2.6v3 | | | | |

The biophysical properties of LNP particles were characterized using a dynamic light scattering instrument as exemplified below.

TABLE 11

| | N/P | Size, | | Z-potential, | STD, |
|---|---|---|---|---|---|
| LNP ID | ratio | nm | PDI | mV | mV |
| LNP102-npRB2.6v7 | 7 | 83.37 | 0.04 | −10.9 | 0.9 |
| LNP102-npRB2.6v3 | 7 | 82.4 | 0.08 | −11.7 | 0.8 |

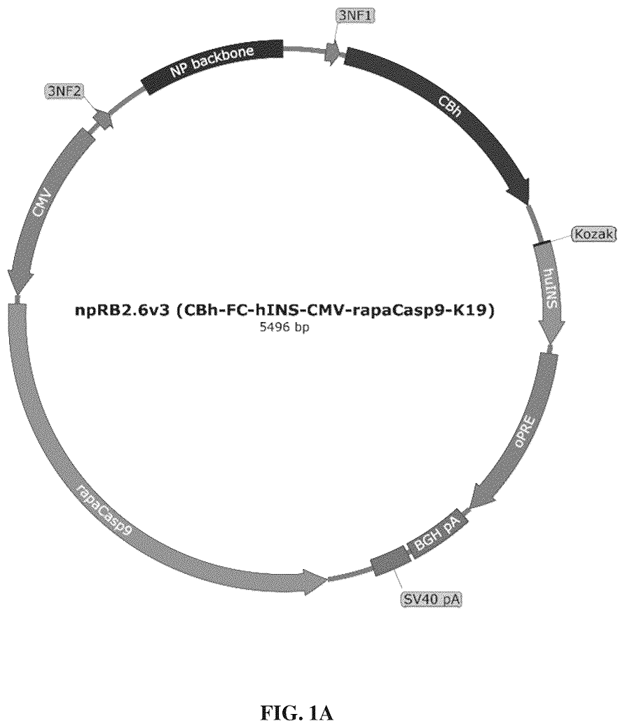

The genetic constructs shown in FIGS. 1A-1B were encapsulated in the formulations at N/P ratios of 4 to 10. The formulations were subsequently used to treat primary human adipocytes and pre-adipocytes at doses of 50-500 ng/well. Both constructs contain a suicide gene down-titration genetic element consisting of the rapaCasp9 gene fused to the K19 aptazyme that requires the presence of two regulators, Tetracycline and Rapamycin, for activation.

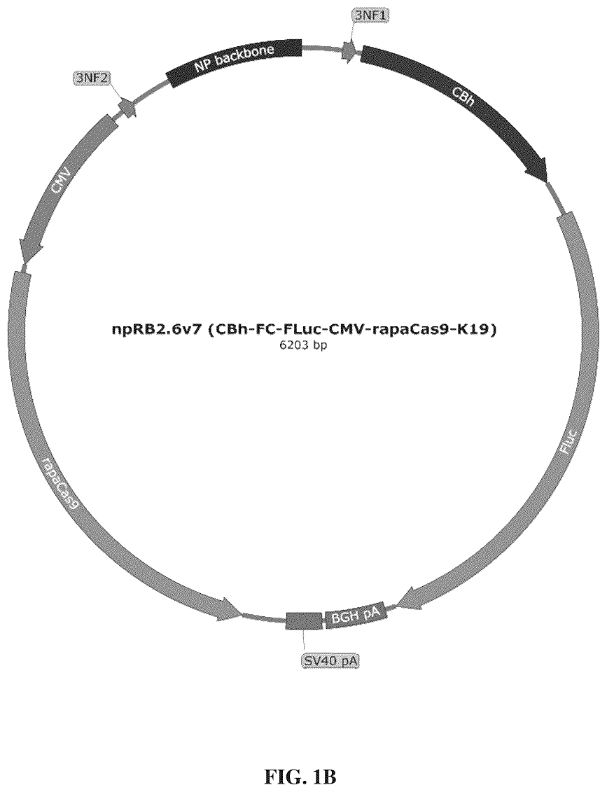

The firefly-luciferase reporter gene expressed from primary human adipocytes transduced with LNP102-npRB2.6v7 was used to quantify the effect of a single regulator, Rapamycin and the response of the Rapamycin inducible suicide gene down-titration element, while the effective dose of the other regulator, Tetracycline, was fixed at 0.1 mM. Specifically, a mock negative control was used to assess absence of luminescence (no cells expressing fLuc). A 0 nM Rapamycin positive control was used to assess baseline expression of fLuc without down titration. Rapamycin concentration was increased from 0 nM to 0.1 nM and a clear dose dependent reduction in transgene expression as observed using quantitative bioluminescence analysis. As such, the induction of the down-titration suicide gene element reduced expression of the reporter gene in a dose dependent manner, within 76 hours of inducer activation (FIG. 2).

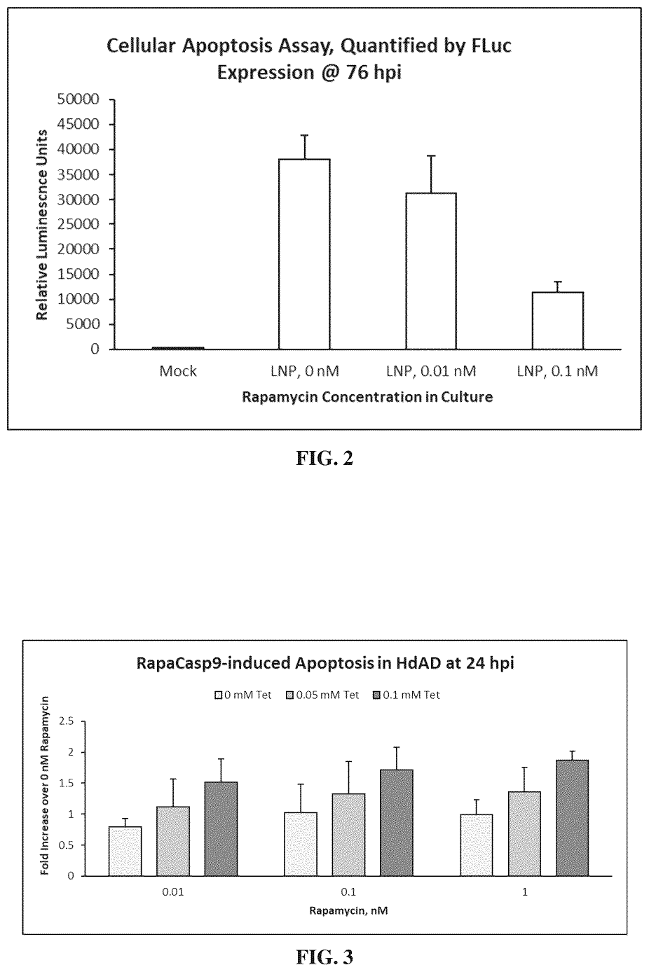

In parallel, the Annexin NanoBiT, 500× bioluminescence assay was used to measure functionality of construct down-titratability/downward adjustment in primary human adipocyte culture transduced with LNP102-npRB2.6v3. Rapamycin administration to cultured cells ranged from 0.01 to 1 nM, while the second regulator, Tetracycline was added at a concentration of 0 mM, 0.05 mM, or 0.1 mM. While no induction of apoptosis is observed in the absence of tetracycline indicating functionality of the aptazyme, a clear, dose dependent cellular apoptosis is observed following dosing of Tetracycline at concentrations of 0.05 and 0.1 mM for all Rapamycin concentrations. Following apoptosis, expression from the therapeutic transgene is effectively eliminated, enabling downward titration of the gene therapy (FIG. 3).

### Example 6

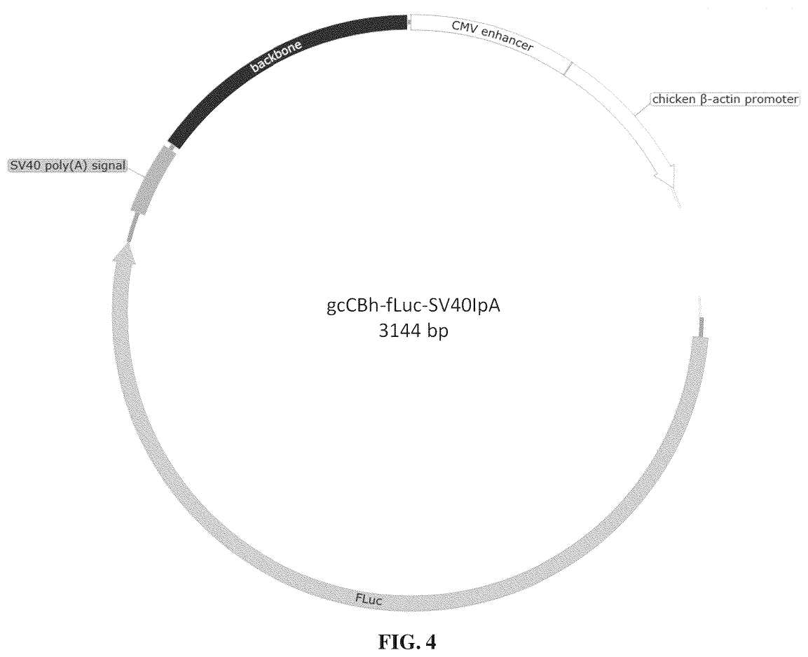

A lipid-based carrier formulation was prepared and mixed at an N/P ratio of 7 for subsequent administration to C57BL/6 mice or C6 Albino background mice. A total of 2-10 ug of formulation was injected into the mice as the “Initial Dose” or “Up-Titration Doses”, which were administered 2-4 weeks following the administration of the Initial Dose to allow for any potential formation of an immune response. Administered construct sizes varied between 6203 and 3144 bp and included the constructs shown in FIG. 1B and FIG. 4.

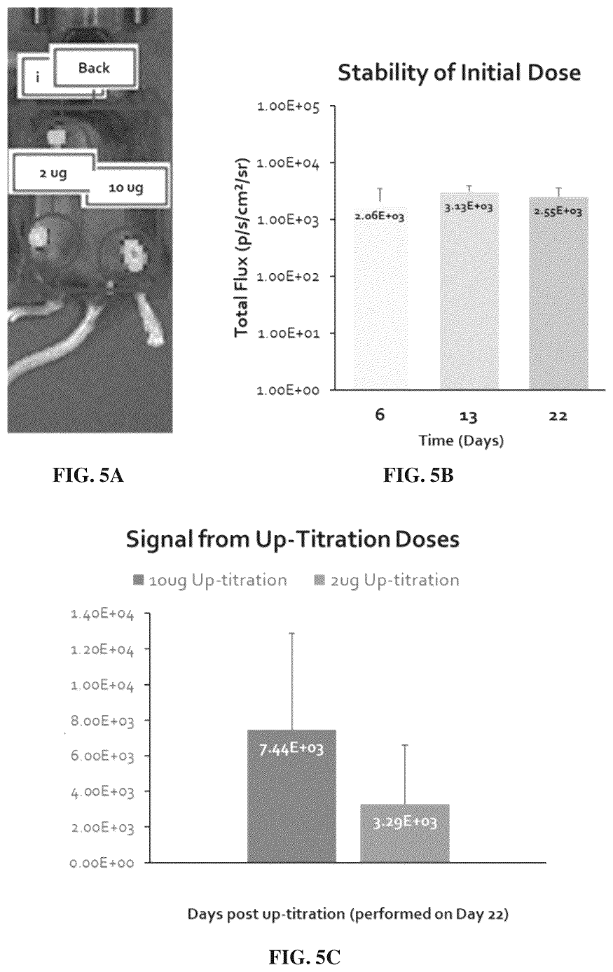

Following subcutaneous injection of the initial dose, the animals were monitored for serious adverse events, general safety, and durability of the signal (reporter transgene expression) using in vivo bioluminescence imaging (IVIS). IVIS confirmed the durability of the initial signal (transgene expression) through Day 22 (FIG. 5A and FIG. 5B), and the ability to up-titrate the initial dose in a predictable dose-dependent manner, allowing for not only re-dosing, but also controlled up-titratability of the initial treatment as shown in FIG. 5A and FIG. 5C.

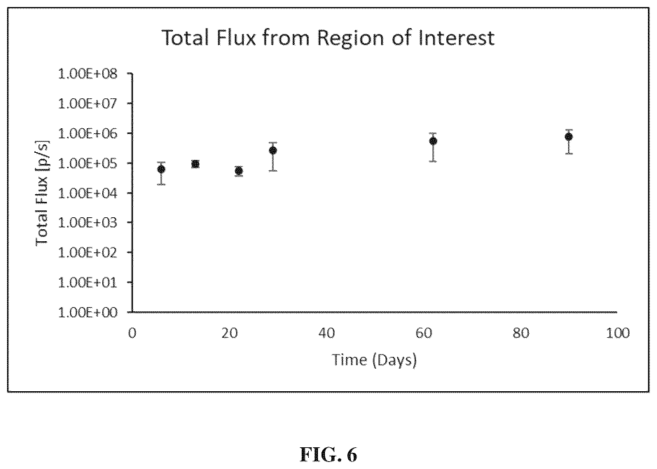

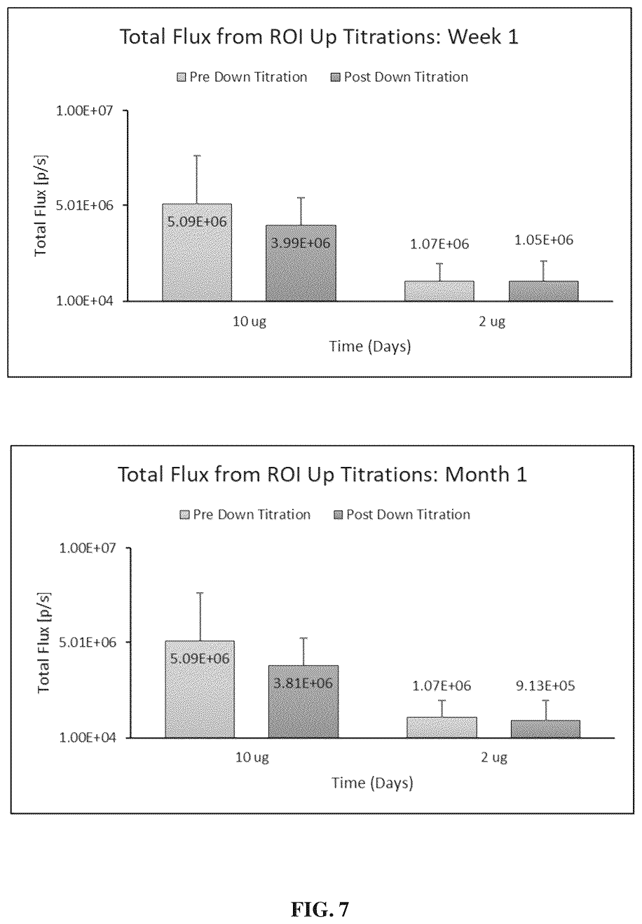

The combined treatments were subsequently followed to evaluate long-term durability of reporter transgene expression. Over a 90-day period, IVIS measurements were performed a total of 6 times. At all times the signal was clearly observed localized to the original injection site and at a level statistically exceeding background luminescence indicating a high stability and excellent long-term durability potential of the gene therapy (FIGS. 6-7).

The present example illustrates a minimum observable down-titration, which ranges between ˜2 and ˜15% and may be subsequently increased following additional stimuli administration.

### Example 7

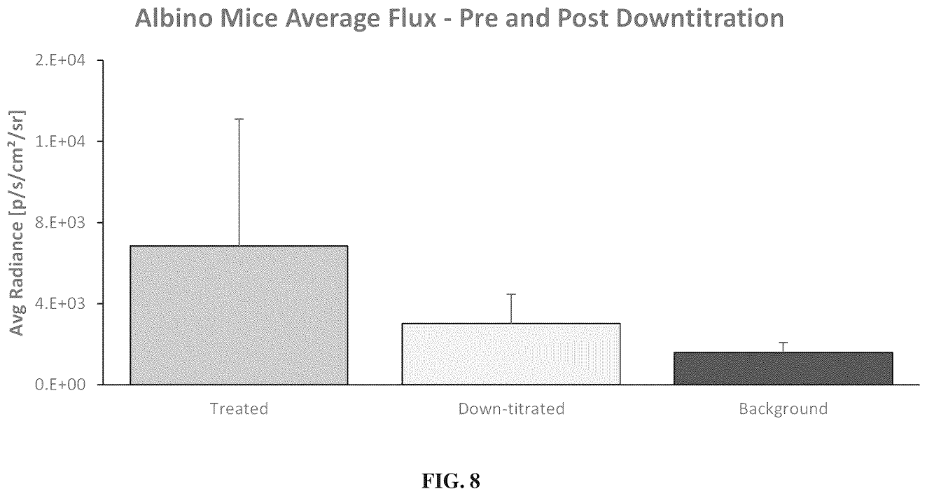

In the present example 3 ug of an LNP formulation N/P7 prepared as a 4 mL batch of 144 uL of 8-[(2-hydroxyethyl) [6-oxo-6-(undecyloxy) hexyl]amino]-octanoic acid, 1-octylnonyl ester, 128 uL of 1,2-Distearoylphosphatidylcholine, 1184 uL of cholesterol, 1512 uL of 1,2-dimyristoyl-rac-glycero-3-methoxy-Polyethylene glycol (2,000), 1032 uL of ethanol, containing a 5496nt genetic construct encoding a firefly Luciferase reporter gene under the regulation of a CBh promoter (hybrid cytomegalovirus enhancer/chicken β-actin) was delivered subcutaneously to C57BL/6 Albino mice. Following 3 months of imaging to ensure signal stability animals were subjected to down-titration of signal via cryolipolysis using a 1.5 cm\hat{ }2 diameter probe delivering a heat flux of 3 W/cm\hat{ }2 at 0-degrees Celsius (a flux previously confirmed to enable cooling of the subcutaneous tissues to approximately 6 degrees Celsius) (FIG. 8). A series of 3 bi-daily treatments were administered for a total cooling time of 10 minutes following equilibration of probe and skin. Glycerol was applied to the probe tip to prevent crystal formation and potential damage to the dermis. The fat cell reduction can be extrapolated to the change in therapeutic transgene expression following treatment. Additional treatment may be sequentially applied to further reduce the subcutaneous adipose cell count to completely shut off gene expression if required.

The reporter gene can be replaced by any therapeutically relevant transgene, which has been qualified or is in clinical evaluation assessing therapeutic efficacy via subcutaneous delivery as a protein or mRNA. The dose, formulation, concentration, number of injections, promoter, or other regulatory sequences can be adjusted to optimize therapeutic efficacy of the transgene. The flux, probe area, time, and cooling/heating profile as well as cooling auxiliaries can be adjusted to achieve cryolipolysis in non-rodent tissues if the treatment is administered to, for example, companion animals, humans, or horses.

### Example 8

The present example describes a method of down-titration using the Lox-Cre system:

Genetic Construct Sequence (excluding plasmid backbone):
(SEQ ID NO: 56)
ctggggactttccagcctggggactttccagctgggactttccaggGGCTCCGGTGCCCGTCAGTGGG
CAGAGCGCACATCGCCCACAGTCCCCGAGAAGITGGGGGGAGGGGTCGGCAATTGATCCGGTGCCTAG
AGAAGGTGGCGCGGGGTAAACTGGGAAAGTGATGTCGTGTACTGGCTCCGCCTTTTTCCCGAGGGTGG
GGGAGAACCGTATATAAGTGCAGTAGTCGCCGTGAACGTTCTTTTTCGCAACGGGTTTGCCGCCAGAA
CACAGGATAACTTCGTATAgcatacatTATACGAAGTTATgttggacgcgtcgctagcgccaccATGG
TCTTCACACTCGAAGATTTCGTTGGGGACTGGCGACAGACAGCCGGCTACAACCTGGACCAAGTCCTT
GAACAGGGAGGTGTGTCCAGTTTGTTTCAGAATCTCGGGGTGTCCGTAACTCCGATCCAAAGGATTGT
CCTGAGCGGTGAAAATGGGCTGAAGATCGACATCCATGTCATCATCCCGTATGAAGGTCTGAGCGGCG
ACCAAATGGGCCAGATCGAAAAAATTTTTAAGGTGGTGTACCCTGTGGATGATCATCACTTTAAGGTG
ATCCTGCACTATGGCACACTGGTAATCGACGGGGTTACGCCGAACATGATCGACTATTTCGGACGGCC
GTATGAAGGCATCGCCGTGTTCGACGGCAAAAAGATCACTGTAACAGGGACCCTGTGGAACGGCAACA
AAATTATCGACGAGCGCCTGATCAACCCCGACGGCTCCCTGCTGTTCCGAGTAACCATCAACGGAGTG
ACCGGCTGGCGGCTGTGCGAACGCATTCTGGCGTAAtgactgtgccttctagttgccagccatctgtt
gtttgcccctcccccgtgccttccttgaccctggaaggtgccactcccactgtcctttcctaataaaa
tgaggaaattgcatcgcattgtctgagtaggtgtcattctattctggggggtggggtggggcaggaca
gcaagggggaggattgggaagagaatagcaggcaggctggggagggccgccgatcgaacttgtttatt
gcagcttataatggttacaaataaagcaatagcatcacaaatttcacaaataaagcatttttttcact
gcattctagttgtggtttgtccaaactcatcaatgtatcttatttgtttgtttgagcgcgtttcgtcc
tatttgggactcatcagctggatgtagtaggtggtcgtattcttcacctctccagatcgaaatctggt
atgttttaccacgaatccaggacgcgcctttgtttgtttggactagaagcggccgcctaTCAAGCIGT
GGCAGGGAAACCCTCTGCCTCCCCCGTGATGTAATACTTTTGCAAGGAATGCGATGAAGTAGAGCCCG
CAGTGGCCAAGTGGCTTTGGTCCGTCTCCTCCACGGATGCCCCTCCACGGCTAGTGGGCGCATGTAGG
CGGTGGGCGTCCGCCGCCTCCAGCAGCAGGTCATAGAGGGGCACCACGTTCTTGCACTTCATGCTGTA
CAGATGCTCCATGCCTTTGTTACTCATGTGCCTGATGTGGGAGAGGATGAGGAGGAGCTGGGCCAGCC
GCTGGTGCTGCTGCTGCAGGGTCAGGCCTGCCTTGGCCATCAGGTGGATCAAAGTGTCTGTGATCTTG
TCCAGGACTCGGTGGATATGGTCCTTCTCTTCCAGAGACTTCAGGGTGCTGGACAGAAATGTGTACAC
TCCAGAATTAAGCAAAATAATAGATTTGAGGCACACAAACTCCTCTCCCTGCAGATTCATCATGCGGA
ACCGAGATGATGTAGCCAGCAGCATGTCGAAGATCTCCACCATGCCCTCTACACATTTTCCCTGGTTC
CTGTCCAAGAGCAAGTTAGGAGCAAACAGTAGCTTCACTGGGTGCTCCATGGAGCGCCAGACGAGACC
AATCATCAGGATCTCTAGCCAGGCACATTCTAGAAGGTGGACCTGATCATGGAGGGTCAAATCCACAA
AGCCTGGCACCCTCTTCGCCCAGTTGATCATGTGAACCAGCTCCCTGTCTGCCAGGTTGGTCAGTAAG
CCCATCATCGAAGCTTCACTGAAGGGTCTGGTAGGATCATACTCGGAATAGAGTATGGGGGGCTCAGC
ATCCAACAAGGCACTGACCATCTGGTCGGCCGTCAGGGACAAGGCCAGGCTGTTCTTCTTAGAGCGTT
TGATCATGAGCGGGCTTGGCCAAAGGTTGGCAGCTCTCATGTCTCCAGCAGATGGCTCGAGATCGCCA
TCTTCCAGCAGGCGCACCATTGCCCCTGTTTCACTATCCAGGTTACGGATATAGTTCATGACAATATT
TACATTGGTCCAGCCACCAGCTTGCATGATCTCCGGTATTGAAACTCCAGCGCGGGCCATATCTCGCG
CGGCTCCGACACGGGCACTGTGTCCAGACCAGGCCAGGTATCTCTGACCAGAGTCATCCTTAGCGCCG
TAAATCAATCGATGAGTTGCTTCAAAAATCCCTTCCAGGGCGCGAGTTGATAGCTGGCTGGTGGCAGA
TGGCGCGGCAACACCATTTTTTCTGACCCGGCAAAACAGGTAGITATTCGGATCATCAGCTACACCAG
AGACGGAAATCCATCGCTCGACCAGTTTAGTTACCCCCAGGCTAAGTGCCTTCTCTACACCTGCGGTG
CTAACCAGCGTTTTCGTTCTGCCAATATGGATTAACATTCTCCCACCGTCAGTACGTGAGATATCTTT
AACCCTGATCCTGGCAATTTCGGCTATACGTAACAGGGTGTTATAAGCAATCCCCAGAAATGCCAGAT
TACGTATATCCTGGCAGCGATCGCTATTTTCCATGAGTGAACGAACCTGGTCGAAATCAGTGCGTTCG
AACGCTAGAGCCTGTTTTGCACGTTCACCGGCATCAACGTTTTCTTTTCGGATCCGCCGCATAACCAG
TGAAACAGCATTGCTGTCACTTGGTCGTGGCAGCCCGGACCGACGATGAAGCATGTTTAGCTGGCCCA
AATGTTGCTGGATAGTTTTTACTGCCAGACCGCGCGCCTGAAGATATAGAAGATAATCGCGAACATCT
TCAGGTTCTGCGGGAAACCATTTCCGGTTATTCAACTTGCACCATGCCGCCCACGACCGGCAAACGGA
CAGAAGCATTTTCCAGGTATGCTCAGAAAACGCCTGGCGATCCCTGAACATGTCCATCAGGTTCTTGC
GAACCTCATCACTCGTTGCATCGACCGGTAATGCAGGCAAATTTTGGTGTACGGTCAGTAAATTGGAC
ATggtggcagcctgcttttttgtacaaacttgATAACTTCGTATAgcatacatTATACGAAGTTATCC
TGTGTTCTGGCGGCAAACCCGTTGCGAAAAAGAACGTTCACGGCGACTACTGCACTTATATACGGTTC
TCCCCCACCCTCGGGAAAAAGGCGGAGCCAGTACACGACATCACTTTCCCAGTTTACCCCGCGCCACC
TTCTCTAGGCACCGGATCAATTGCCGACCCCTCCCCCCAACTTCTCGGGGACTGTGGGCGATGTGCGC
TCTGCCCACTGACGGGCACCGGAGCCctggggactttccagctggggactttccagctgggactttcc
aggag
3NF1 DTS:
(SEQ ID NO: 7)
Ctggggactttccagcctggggactttccagctgggactttccagg
3NF2 DTS:
(SEQ ID NO: 8)
ctggggactttccagctggggactttccagctgggactttccaggag
EFS promoter:
(SEQ ID NO: 3)
GGCTCCGGTGCCCGTCAGTGGGCAGAGCGCACATCGCCCACAGTCCCCGAGAAGTTGGGGGGAGGGGT
CGGCAATTGATCCGGTGCCTAGAGAAGGTGGCGCGGGGTAAACTGGGAAAGTGATGTCGTGTACTGGC
TCCGCCTTTTTCCCGAGGGTGGGGGAGAACCGTATATAAGTGCAGTAGTCGCCGTGAACGTTCTTTTT
CGCAACGGGTTTGCCGCCAGAACACAGG
loxP Site:
(SEQ ID NO: 57)
ATAACTTCGTATAgcatacatTATACGAAGTTAT
NLuc Coding Sequence:
(SEQ ID NO: 58)
ATGGTCTTCACACTCGAAGATTTCGTTGGGGACTGGCGACAGACAGCCGGCTACAACCTGGACCAAGT
CCTTGAACAGGGAGGTGTGTCCAGTTTGTTTCAGAATCTCGGGGTGTCCGTAACTCCGATCCAAAGGA
TTGTCCTGAGCGGTGAAAATGGGCTGAAGATCGACATCCATGTCATCATCCCGTATGAAGGTCTGAGC
GGCGACCAAATGGGCCAGATCGAAAAAATTTTTAAGGTGGTGTACCCTGTGGATGATCATCACTTTAA
GGTGATCCTGCACTATGGCACACTGGTAATCGACGGGGTTACGCCGAACATGATCGACTATTTCGGAC
GGCCGTATGAAGGCATCGCCGTGTTCGACGGCAAAAAGATCACTGTAACAGGGACCCTGTGGAACGGC
AACAAAATTATCGACGAGCGCCTGATCAACCCCGACGGCTCCCTGCTGTTCCGAGTAACCATCAACGG
AGTGACCGGCTGGCGGCTGTGCGAACGCATTCTGGCGTAA
NLuc Amino Acid Sequence:
(SEQ ID NO: 59)
MVFTLEDFVGDWRQTAGYNLDQVLEQGGVSSLFQNLGVSVTPIQRIVLSGENGLKIDIHVIIPYEGLS
GDQMGQIEKIFKVVYPVDDHHFKVILHYGTLVIDGVTPNMIDYFGRPYEGIAVFDGKKITVTGTLWNG
NKIIDERLINPDGSLLFRVTINGVTGWRLCERILA
BGH poly A Signal:
(SEQ ID NO: 60)
Ctgtgccttctagttgccagccatctgttgtttgcccctcccccgtgccttccttgaccctggaaggt
gccactcccactgtcctttcctaataaaatgaggaaattgcatcgcattgtctgagtaggtgtcattc
tattctggggggtggggtggggcaggacagcaagggggaggattgggaagagaatagcaggcaggctg
ggga
Cre-ERT2 Coding Sequence:
(SEQ ID NO: 61)
ATGTCCAATTTACTGACCGTACACCAAAATTTGCCTGCATTACCGGTCGATGCAACGAGTGATGAGGT
TCGCAAGAACCTGATGGACATGTTCAGGGATCGCCAGGCGTTTTCTGAGCATACCTGGAAAATGCTTC
TGTCCGTTTGCCGGTCGTGGGCGGCATGGTGCAAGTTGAATAACCGGAAATGGTTTCCCGCAGAACCT
GAAGATGTTCGCGATTATCTTCTATATCTTCAGGCGCGCGGTCTGGCAGTAAAAACTATCCAGCAACA
TTTGGGCCAGCTAAACATGCTTCATCGTCGGTCCGGGCTGCCACGACCAAGTGACAGCAATGCTGTTT
CACTGGTTATGCGGCGGATCCGAAAAGAAAACGTTGATGCCGGTGAACGTGCAAAACAGGCTCTAGCG
TTCGAACGCACTGATTTCGACCAGGTTCGTTCACTCATGGAAAATAGCGATCGCTGCCAGGATATACG
TAATCTGGCATTTCTGGGGATTGCTTATAACACCCTGTTACGTATAGCCGAAATTGCCAGGATCAGGG
TTAAAGATATCTCACGTACTGACGGTGGGAGAATGTTAATCCATATTGGCAGAACGAAAACGCTGGTT
AGCACCGCAGGTGTAGAGAAGGCACTTAGCCTGGGGGTAACTAAACTGGTCGAGCGATGGATTTCCGT
CTCTGGTGTAGCTGATGATCCGAATAACTACCTGTTTTGCCGGGTCAGAAAAAATGGTGTTGCCGCGC
CATCTGCCACCAGCCAGCTATCAACTCGCGCCCTGGAAGGGATTTTTGAAGCAACTCATCGATTGATT
TACGGCGCTAAGGATGACTCTGGTCAGAGATACCTGGCCTGGTCTGGACACAGTGCCCGTGTCGGAGC
CGCGCGAGATATGGCCCGCGCTGGAGTTTCAATACCGGAGATCATGCAAGCTGGTGGCTGGACCAATG
TAAATATTGTCATGAACTATATCCGTAACCTGGATAGTGAAACAGGGGCAATGGTGCGCCTGCTGGAA
GATGGCGATCTCGAGCCATCTGCTGGAGACATGAGAGCTGCCAACCTTTGGCCAAGCCCGCTCATGAT
CAAACGCTCTAAGAAGAACAGCCTGGCCTTGTCCCTGACGGCCGACCAGATGGTCAGTGCCTTGTTGG
ATGCTGAGCCCCCCATACTCTATTCCGAGTATGATCCTACCAGACCCTTCAGTGAAGCTTCGATGATG
GGCITACTGACCAACCTGGCAGACAGGGAGCTGGTTCACATGATCAACTGGGCGAAGAGGGTGCCAGG
CTTTGTGGATTTGACCCTCCATGATCAGGTCCACCTTCTAGAATGTGCCTGGCTAGAGATCCTGATGA
TTGGTCTCGTCTGGCGCTCCATGGAGCACCCAGTGAAGCTACTGTTTGCTCCTAACTTGCTCTTGGAC
AGGAACCAGGGAAAATGTGTAGAGGGCATGGTGGAGATCTTCGACATGCTGCTGGCTACATCATCTCG
GTTCCGCATGATGAATCTGCAGGGAGAGGAGTTTGTGTGCCTCAAATCTATTATTTTGCTTAATTCTG
GAGTGTACACATTTCTGTCCAGCACCCTGAAGTCTCTGGAAGAGAAGGACCATATCCACCGAGTCCTG
GACAAGATCACAGACACTTTGATCCACCTGATGGCCAAGGCAGGCCTGACCCTGCAGCAGCAGCACCA
GCGGCTGGCCCAGCTCCTCCTCATCCTCTCCCACATCAGGCACATGAGTAACAAAGGCATGGAGCATC
TGTACAGCATGAAGTGCAAGAACGTGGTGCCCCTCTATGACCTGCTGCTGGAGGCGGCGGACGCCCAC
CGCCTACATGCGCCCACTAGCCGTGGAGGGGCATCCGTGGAGGAGACGGACCAAAGCCACTTGGCCAC
TGCGGGCTCTACTTCATCGCATTCCTTGCAAAAGTATTACATCACGGGGGAGGCAGAGGGTTTCCCTG
CCACAGCTTGA
Cre-ERT2 Amino Acid Sequence:
(SEQ ID NO: 11)
MSNLLTVHQNLPALPVDATSDEVRKNLMDMFRDRQAFSEHTWKMLLSVCRSWAAWCKLNNRKWFPAEP
EDVRDYLLYLQARGLAVKTIQQHLGQLNMLHRRSGLPRPSDSNAVSLVMRRIRKENVDAGERAKQALA
FERTDFDQVRSLMENSDRCQDIRNLAFLGIAYNTLLRIAEIARIRVKDISRIDGGRMLIHIGRTKTLV
STAGVEKALSLGVTKLVERWISVSGVADDPNNYLFCRVRKNGVAAPSATSQLSTRALEGIFEATHRLI
YGAKDDSGORYLAWSGHSARVGAARDMARAGVSIPEIMQAGGWTNVNIVMNYIRNLDSETGAMVRLLE
DGDLEPSAGDMRAANLWPSPLMIKRSKKNSLALSLTADQMVSALLDAEPPILYSEYDPTRPFSEASMM
GLLTNLADRELVHMINWAKRVPGFVDLTLHDQVHLLECAWLEILMIGLVWRSMEHPVKLLFAPNLLLD
RNQGKCVEGMVEIFDMLLATSSRFRMMNLQGEEFVCLKSIILLNSGVYTFLSSTLKSLEEKDHIHRVL
DKITDTLIHLMAKAGLILQQQHORLAQLLLILSHIRHMSNKGMEHLYSMKCKNVVPLYDLLLEAADAH
RLHAPTSRGGASVEETDQSHLATAGSTSSHSLQKYYITGEAEGFPATA
Insulator:
(SEQ ID NO: 31)
caaacaaacaaa
K19 aptazyme:
(SEQ ID NO: 32)
ggcgcgtcctggattcgtggtaaaacataccagatttcgatctggagaggtgaagaatacgaccacct
actacatccagctgatgagtcccaaataggacgaaacgcgct
SV40 pA:
(SEQ ID NO: 33)
Taagatacattgatgagtttggacaaaccacaactagaatgcagtgaaaaaaatgctttatttgtgaa
atttgtgatgctattgctttatttgtaaccattataagctgcaataaacaagtt

### Example 9

The present example describes a therapeutic gene therapy for down titration via the application of a temperature reducing device, ultrasound, or electromagnetic radiation, which can impact the gene expression or viability of adipocytes or pre-adipocytes expressing the gene therapy.

Genetic Construct Sequence (excluding plasmid backbone):
(SEQ ID NO: 62)
ctagctggcttgttgtccacaaccattaaaccttaaaagctttaaaagccttatatattctttttttt
cttataaaacttaaaaccttagaggctatttaagttgctgatttatattaattttattgttcaaacat
gagagcttagtacgtgaaacatgagagcttagtacattagccatgagagcttagtacattagccatga
gggtttagttcattaaacatgagagcttagtacattaaacatgagagcttagtacattaaacatgaga
gcttagtacatactatcaacaggttgaactgctgatctgtacagtagaattggtaaagagagttgtgt
aaaatattgagttcgcacatcttgttgtctgattattgatttttggcgaaaccatttgatcatatgac
aagatgtgtatctaccttaacttaatgattttgataaaaatcattaggtaccctggggactttccagc
ctggggactttccagctgggactttccaggcgttacataacttacggtaaatggcccgcctggctgac
cgcccaacgacccccgcccattgacgtcaatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattgtgcccagtacatgaccttatgggactttc
ctacttggcagtacatctacgtattagtcatcgctattaccatggtcgaggtgagccccacgttctgc
ttcactctccccatctcccccccctccccacccccaattttgtatttatttattttttaattattttg
tgcagcgatgggggcggggggggggggggggcgcgcgccaggcggggcggggcggggcgaggggcggg
gcggggcgaggcggagaggtgcggcggcagccaatcagagcggcgcgctccgaaagtttccttttatg
gcgaggcggcggcggcggcggccctataaaaagcgaagcgcgcggcgggcgggagtcgctgcgcgctg
ccttcgccccgtgccccgctccgccgccgcctcgcgccgcccgccccggctctgactgaccgcgttac
tcccacaggtgagcgggcgggacggcccttctcctccgggctgtaattagctgagcaagaggtaaggg
tttaagggatggttggttggtggggtattaatgtttaattacctggagcacctgcctgaaatcacttt
ttttcaggttgggttggacgcgtcgctagcgccaccatggtcttcacactcgaagatttcgttgggga
ctggcgacagacagccggctacaacctggaccaagtccttgaacagggaggtgtgtccagtttgtttc
agaatctcggggtgtccgtaactccgatccaaaggattgtcctgagcggtgaaaatgggctgaagatc
gacatccatgtcatcatcccgtatgaaggtctgagcggcgaccaaatgggccagatcgaaaaaatttt
taaggtggtgtaccctgtggatgatcatcactttaaggtgatcctgcactatggcacactggtaatcg
acggggttacgccgaacatgatcgactatttcggacggccgtatgaaggcatcgccgtgttcgacggc
aaaaagatcactgtaacagggaccctgtggaacggcaacaaaattatcgacgagcgcctgatcaaccc
cgacggctccctgctgttccgagtaaccatcaacggagtgaccggctggcggctgtgcgaacgcattc
tggcgtaatgactgtgccttctagttgccagccatctgttgtttgcccctcccccgtgccttccttga
ccctggaaggtgccactcccactgtcctttcctaataaaatgaggaaattgcatcgcattgtctgagt
aggtgtcattctattctggggggtggggtggggcaggacagcaagggggaggattgggaagagaatag
caggcaggctggggactggggactttccagctggggactttccagctgggactttccaggaga
3NF1 DTS:
(SEQ ID NO: 7)
Ctggggactttccagcctggggactttccagctgggactttccagg
3NF2 DTS:
(SEQ ID NO: 8)
ctggggactttccagctggggactttccagctgggactttccaggag
CBh Promoter:
(SEQ ID NO: 2)
Cgttacataacttacggtaaatggcccgcctggctgaccgcccaacgacccccgcccattgacgtcaa
tagtaacgccaatagggactttccattgacgtcaatgggtggagtatttacggtaaactgcccacttg
gcagtacatcaagtgtatcatatgccaagtacgccccctattgacgtcaatgacggtaaatggcccgc
ctggcattgtgcccagtacatgaccttatgggactttcctacttggcagtacatctacgtattagtca
tcgctattaccatggtcgaggtgagccccacgttctgcttcactctccccatctcccccccctcccca
cccccaattttgtatttatttattttttaattattttgtgcagcgatgggggcggggggggggggggg
gcgcgcgccaggcggggcggggcggggcgaggggcggggcggggcgaggcggagaggtgcggcggcag
ccaatcagagcggcgcgctccgaaagtttccttttatggcgaggcggcggcggcggcggccctataaa
aagcgaagcgcgcggcgggcgggagtcgctgcgcgctgccttcgccccgtgccccgctccgccgccgc
ctcgcgccgcccgccccggctctgactgaccgcgttactcccacaggtgagcgggcgggacggccctt
ctcctccgggctgtaattagctgagcaagaggtaagggtttaagggatggttggttggtggggtatta
atgtttaattacctggagcacctgcctgaaatcactttttttcaggttgg
NLuc Coding Sequence:
(SEQ ID NO: 58)
Atggtcttcacactcgaagatttcgttggggactggcgacagacagccggctacaacctggaccaagt
ccttgaacagggaggtgtgtccagtttgtttcagaatctcggggtgtccgtaactccgatccaaagga
ttgtcctgagcggtgaaaatgggctgaagatcgacatccatgtcatcatcccgtatgaaggtctgagc
ggcgaccaaatgggccagatcgaaaaaatttttaaggtggtgtaccctgtggatgatcatcactttaa
ggtgatcctgcactatggcacactggtaatcgacggggttacgccgaacatgatcgactatttcggac
ggccgtatgaaggcatcgccgtgttcgacggcaaaaagatcactgtaacagggaccctgtggaacggc
aacaaaattatcgacgagcgcctgatcaaccccgacggctccctgctgttccgagtaaccatcaacgg
agtgaccggctggcggctgtgcgaacgcattctggcgtaa
NLuc Amino Acid Sequence:
(SEQ ID NO: 59)
MVFTLEDFVGDWRQTAGYNLDQVLEQGGVSSLFQNLGVSVTPIQRIVLSGENGLKIDIHVIIPYEGLS
GDQMGQIEKIFKVVYPVDDHHFKVILHYGTLVIDGVTPNMIDYFGRPYEGIAVFDGKKITVTGTLWNG
NKIIDERLINPDGSLLFRVTINGVTGWRLCERILA
BGH poly A Signal:
(SEQ ID NO: 60)
ctgtgccttctagttgccagccatctgttgtttgcccctcccccgtgccttccttgaccctggaaggt
gccactcccactgtcctttcctaataaaatgaggaaattgcatcgcattgtctgagtaggtgtcattc
tattctggggggtggggtggggcaggacagcaagggggaggattgggaagagaatagcaggcaggctg
ggga

### Example 10

The present example describes a means of down-titration or downregulation of gene expression from the therapeutic construct via the embedding of targeting epigenetic silencers, the expression and/or activation of which may be induced by the administration of one or more exogenous stimuli.

Specifically, a human or other target species (as an example Mouse) DNA methyl transferase (or other epigenetic silencer) with an example sequence:

Human DNMT3A (CD):
(SEQ ID NO: 63)
NHDQEFDPPKVYPPVPAEKRKPIRVLSLFDGIATGLLVLKDLGIQVDRYIASEVCEDSITVGMVRHQG
KIMYVGDVRSVTQKHIQEWGPFDLVIGGSPCNDLSIVNPARKGLYEGTGRLFFEFYRLLHDARPKEGD
DRPFFWLFENVVAMGVSDKRDISRFLESNPVMIDAKEVSAAHRARYFWGNLPGMNRPLASTVNDKLEL
QECLEHGRIAKFSKVRTITTRSNSIKQGKDQHFPVFMNEKEDILWCTEMERVFGFPVHYTDVSNMSRL
ARQRLLGRSWSVPVIRHLFAPLKEYFACV
OR
Mouse DNMT3L (CD):
(SEQ ID NO: 64)
GPMEIYKTVSAWKRQPVRVLSLFRNIDKVLKSLGFLESGSGSGGGTLKYVEDVINVVRRDVEKWGPFD
LVYGSTQPLGSSCDRCPGWYMFQFHRILQYALPRQESQRPFFWIFMDNLLLTEDDQETTTRFLQTEAV
TLQDVRGRDYQNAMRVWSNIPGLKSKHAPLTPKEEEYLQAQVRSRSKLDAPKVDLLVKNCLLPLREYF
KYFSQNSLPL

Is coupled with a targeting element such as a zinc-finger motif or protein with an example sequence of:

ZN627_Hu:
(SEQ ID NO: 65)
DSVAFEDVAVNFTLEEWALLDPSQKNLYRDVMRETFRNLASVGKQWEDQ
NIEDPFKIPRRNISHIPERLCESKEGGQGEE

Which may optionally be combined with NLS sequences such as: MAPKKKRKV (SEQ ID NO: 66) and flexible spacer or linker sequences such as SGGGGSGGGGS (SEQ ID NO: 67) to produce the following fusion protein:

hD3A(CD)-mD3L(CD)-ZF(NLucTSS34)-ZN627:
(SEQ ID NO: 12)
MAPKKKRKVNHDQEFDPPKVYPPVPAEKRKPIRVLSLFDGIATGLLVLKDLGIQVDRYIASEVCEDSI
TVGMVRHQGKIMYVGDVRSVTQKHIQEWGPFDLVIGGSPCNDLSIVNPARKGLYEGTGRLFFEFYRLL
HDARPKEGDDRPFFWLFENVVAMGVSDKRDISRFLESNPVMIDAKEVSAAHRARYFWGNLPGMNRPLA
STVNDKLELQECLEHGRIAKFSKVRTITTRSNSIKQGKDQHFPVFMNEKEDILWCTEMERVFGFPVHY
TDVSNMSRLARQRLLGRSWSVPVIRHLFAPLKEYFACVSSGNSNANSRGPSFSSGLVPLSLRGSHMGP
MEIYKTVSAWKRQPVRVLSLFRNIDKVLKSLGFLESGSGSGGGTLKYVEDVINVVRRDVEKWGPFDLV
YGSTQPLGSSCDRCPGWYMFQFHRILQYALPRQESQRPFFWIEMDNLLLTEDDQETTTRFLQTEAVIL
QDVRGRDYQNAMRVWSNIPGLKSKHAPLTPKEEEYLQAQVRSRSKLDAPKVDLLVKNCLLPLREYFKY
FSQNSLPLSGGGGSGGGGSLEPGEKPYKCPECGKSFSDPGHLVRHQRTHTGEKPYKCPECGKSFSDCR
DLARHQRTHTGEKPYKCPECGKSFSSPADLTRHQRTHTGEKPYKCPECGKSFSRADNLTEHQRTHTGE
KPYKCPECGKSFSQSGHLTEHQRTHTGEKPYKCPECGKSFSRSDHLTTHQRTHTGKKTSSGGGGSGGG
GSDSVAFEDVAVNFTLEEWALLDPSQKNLYRDVMRETFRNLASVGKQWEDQNIEDPFKIPRRNISHIP
ERLCESKEGGQGEE

Which is designed to target the following nucleotide sequence:
Target site (pos 34 downstream TSS): TGGCGACAGACAGCCGGC (SEQ ID NO: 68) as part of the construct sequence:

ZF target region (400 nt total, 200 nt up- and 200 nt downstream TSS):
(SEQ ID NO: 69)
Ccgccccggctctgactgaccgcgttactcccacaggtgagcgggcgggacggcccttctcctccggg
ctgtaattagctgagcaagaggtaagggtttaagggatggttggttggtggggtattaatgtttaatt
acctggagcacctgcctgaaatcactttttttcaggttgggttggacgcgtcgctagcgccaccatgg
tcttcacactcgaagatttcgttggggactggcgacagacagccggctacaacctggaccaagtcctt
gaacagggaggtgtgtccagtttgtttcagaatctcggggtgtccgtaactccgatccaaaggattgt
cctgagcggtgaaaatgggctgaagatcgacatccatgtcatcatcccgtatgaaggtct
ZF:
(SEQ ID NO: 70)
LEPGEKPYKCPECGKSFSDPGHLVRHQRTHTGEKPYKCPECGKSFSDCRDLARHQRTHTGEKPYKCPE
CGKSFSSPADLTRHQRTHTGEKPYKCPECGKSFSRADNLTEHQRTHTGEKPYKCPECGKSFSQSGHLT
EHQRTHTGEKPYKCPECGKSFSRSDHLTTHORTHTGKKTS

Which may be expressed from non-codon optimized or codon optimized sequences such as:

(SEQ ID NO: 71)
ATGGCCCCTAAAAAAAAGAGAAAGGTCAACCACGACCAGGAGTTCGACCCACCTAAGGTGTACCCTCC
TGTGCCAGCCGAGAAGCGGAAGCCAATCCGGGTGCTGAGCCTCTTTGACGGCATCGCCACCGGCCTGC
TGGTGCTGAAGGACCTGGGCATCCAGGTGGACAGATACATCGCCAGTGAAGTGTGCGAAGATTCTATC
ACCGTGGGCATGGTGCGGCACCAAGGCAAGATCATGTACGTGGGCGACGTCCGGTCTGTGACCCAGAA
GCATATCCAGGAGTGGGGCCCCTTCGACCTGGTTATCGGCGGAAGCCCCTGCAACGACCTGAGCATCG
TGAACCCCGCTAGAAAGGGCCTGTACGAGGGCACAGGCAGACTGTTCTTCGAGTTCTACCGGCTGCTG
CACGATGCCCGTCCCAAGGAAGGCGATGACAGACCTTTTTTCTGGCTGTTCGAGAACGTGGTGGCCAT
GGGCGTGAGCGACAAACGGGACATCAGCAGATTCCTGGAGTCTAACCCTGTGATGATTGATGCTAAGG
AAGTGTCTGCCGCCCACCGGGCCCGGTACTTCTGGGGCAACCTGCCAGGCATGAACAGACCTCTCGCC
TCTACCGTGAATGACAAGCTGGAACTGCAGGAGTGTCTGGAACACGGCAGAATCGCCAAGTTCAGTAA
GGTGAGAACCATCACCACCAGGAGCAACAGCATCAAGCAGGGCAAAGATCAGCACTTCCCTGTGTTTA
TGAACGAGAAGGAGGACATCCTGTGGTGCACCGAGATGGAAAGAGTTTTTGGCTTCCCCGTGCACTAC
ACCGACGTTTCTAACATGAGCAGACTGGCCAGACAGCGGCTGCTGGGCAGATCCTGGAGCGTGCCTGT
GATCAGACACCTGTTCGCCCCCCTGAAGGAGTACTTTGCCTGCGTGAGCTCCGGAAATTCTAACGCCA
ACTCCCGCGGCCCCTCCTTCAGCAGCGGACTGGTGCCTCTCAGCCTGAGAGGCAGCCACATGGGACCT
ATGGAAATATACAAGACAGTGTCGGCTTGGAAGAGGCAGCCTGTGAGAGTGCTGAGCCTGTTCAGAAA
CATCGACAAGGTGCTGAAGAGCCTGGGATTTCTGGAATCTGGAAGCGGGTCTGGCGGCGGCACCCTGA
AGTACGTGGAAGATGTGACAAACGTGGIGCGGAGAGATGTCGAGAAATGGGGACCTTTCGATCTGGTG
TACGGCAGCACCCAGCCCCTGGGCAGCAGCTGCGACCGGTGCCCCGGCTGGTACATGTTTCAATTCCA
CCGTATCCTGCAGTACGCCCTGCCTCGGCAGGAAAGCCAGAGACCTTTCTTCTGGATCTTCATGGACA
ACCTGCTGCTGACCGAGGACGACCAGGAGACAACCACAAGATTCCTGCAGACCGAGGCCGTGACCCTG
CAGGATGTCCGGGGCCGCGACTACCAGAATGCCATGCGGGTGTGGAGCAATATCCCTGGCCTGAAGTC
CAAGCACGCCCCTCTGACACCTAAGGAAGAAGAGTACCTGCAAGCTCAGGTGAGAAGCAGATCCAAGC
TGGATGCCCCTAAGGTCGACCTGCTGGTGAAAAACTGCCTGCTGCCACTTAGAGAGTACTTCAAATAC
TTCTCCCAAAATAGCCTGCCCCTGAGCGGCGGAGGCGGGAGCGGCGGAGGCGGCAGCCTGGAGCCTGG
CGAGAAGCCCTACAAGTGCCCAGAGTGCGGCAAATCTTTTAGCGACCCTGGCCACCTGGTGCGGCACC
AGCGGACCCATACCGGTGAGAAACCCTACAAATGTCCTGAATGTGGCAAGAGCTTCTCCGATTGCAGA
GATCTGGCCCGGCACCAGAGAACCCATACAGGCGAAAAACCTTACAAGTGCCCTGAGTGCGGAAAATC
CTTCAGCTCTCCTGCCGACCTGACAAGACACCAGCGGACCCACACAGGCGAGAAACCTTATAAGTGTC
CCGAATGCGGCAAGAGCTTCTCCAGAGCCGATAACCTGACCGAGCACCAAAGGACCCACACCGGCGAA
AAGCCTTATAAGTGCCCAGAATGCGGCAAGAGCTTTTCCCAGAGCGGTCATCTGACAGAGCACCAGAG
AACACACACCGGAGAGAAGCCCTATAAGTGTCCTGAGTGTGGAAAAAGCTTTTCAAGAAGCGACCACC
TCACCACACACCAGCGCACACACACAGGCAAGAAGACCAGCAGCGGTGGCGGCGGCTCTGGCGGCGGA
GGAAGCGACAGCGTGGCCTTCGAGGACGTGGCTGTGAATTTCACTCTGGAAGAGTGGGCTCTGCTCGA
CCCTAGCCAAAAGAACCTGTACAGAGACGTGATGAGAGAAACCTTCAGAAACCTGGCTAGCGTCGGCA
AACAGTGGGAGGATCAGAACATCGAGGATCCTTTCAAGATCCCGAGACGGAACATCAGCCACATCCCA
GAGAGACTGTGCGAGTCCAAGGAGGGAGGCCAGGGCGAGGAATAA

Or alternative examples of DNMT3A-3L-ZF-KRAB fusion:

(SEQ ID NO: 72)
MAPKKKRKVMNHDQEFDPPKVYPPVPAEKRKPIRVLSLFDGIATGLLVLKDLGIQVDRYIASEVCEDS
ITVGMVRHQGKIMYVGDVRSVTQKHIQEWGPFDLVIGGSPCNDLSIVNPARKGLYEGTGRLFFEFYRL
LHDARPKEGDDRPFFWLFENVVAMGVSDKRDISRFLESNPVMIDAKEVSAAHRARYFWGNLPGMNRPL
ASTVNDKLELQECLEHGRIAKFSKVRTITTRSNSIKQGKDQHFPVEMNEKEDILWCTEMERVFGFPVH
YTDVSNMSRLARQRLLGRSWSVPVIRHLFAPLKEYFACVSSGNSNANSRGPSFSSGLVPLSLRGSHMG
PMEIYKTVSAWKRQPVRVLSLFRNIDKVLKSLGFLESGSGSGGGTLKYVEDVINVVRRDVEKWGPFDL
VYGSTQPLGSSCDRCPGWYMFQFHRILQYALPRQESQRPFFWIFMDNLLLTEDDQETTTRFLQTEAVT
LQDVRGRDYQNAMRVWSNIPGLKSKHAPLTPKEEEYLQAQVRSRSKLDAPKVDLLVKNCLLPLREYFK
YFSQNSLPLSGGGGSGGGGSVGIHGVPSRPGERPFQCRICMRNFSHKSSLTRHTRTHTGEKPFQCRIC
MRNFSRTEHLARHLRTHTGSQKPFQCRICMRNFSQSAHLKRHTRTHTGEKPFQCRICMRNFSRTEHLA
RHLRTHTGGGGSQKPFQCRICMRNFSHKSSLTRHTRTHTGEKPFQCRICMRNFSRPESLAPHLRTHLR
GSGGGSMDAKSLIAWSRTLVTFKDVFVDFTREEWKLLDTAQQIVYRNVMLENYKNLVSLGYQLIKPDV
ILRLEKGEEPWLVEREIHQETHPDSETAFEIKSSV
DNMT3A:
(SEQ ID NO: 73)
MNHDQEFDPPKVYPPVPAEKRKPIRVLSLEDGIATGLLVLKDLGIQVDRYIASEVCEDSITVGMVRHQ
GKIMYVGDVRSVTQKHIQEWGPFDLVIGGSPCNDLSIVNPARKGLYEGTGRLFFEFYRLLHDARPKEG
DDRPFFWLFENVVAMGVSDKRDISRFLESNPVMIDAKEVSAAHRARYFWGNLPGMNRPLASTVNDKLE
LQECLEHGRIAKFSKVRTITTRSNSIKQGKDQHFPVFMNEKEDILWCTEMERVFGFPVHYTDVSNMSR
LARORLLGRSWSVPVIRHLFAPLKEYFACV
DNMT3L:
(SEQ ID NO: 74)
SSGNSNANSRGPSFSSGLVPLSLRGSHMGPMEIYKTVSAWKRQPVRVLSLFRNIDKVLKSLGFLESGS
GSGGGTLKYVEDVTNVVRRDVEKWGPFDLVYGSTQPLGSSCDRCPGWYMFQFHRILQYALPRQESQRP
FFWIFMDNLLLTEDDQETTTRFLQTEAVTLQDVRGRDYQNAMRVWSNIPGLKSKHAPLTPKEEEYLQA
QVRSRSKLDAPKVDLLVKNCLLPLREYFKYFSQNSLPLSGGGGSGGGGSVGIHGVP
Linker
(GGGS (SEQ ID NO: 75)),
SV40 NLS
(PKKKRKV (SEQ ID NO: 76)),
ZF
(SRPGERPFQCRICMRNFSHKSSLTRHTRTHTGEKPFQCRICMRNFSRTEHLARHLRTHTGSQKPFQC
RICMRNFSQSAHLKRHTRTHTGEKPFQCRICMRNFSRTEHLARHLRTHTGGGGSQKPFQCRICMRNFS
HKSSLTRHTRTHTGEKPFQCRICMRNFSRPESLAPHLRTHLRGS (SEQ ID NO: 77))

For a combined sequence of D3A/L-dSpCas9-XTEN16-ZN627:

(SEQ ID NO: 78)
MNHDQEFDPPKVYPPVPAEKRKPIRVLSLFDGIATGLLVLKDLGIQVDRYIASEVCEDSITVGMVRHQ
GKIMYVGDVRSVTQKHIQEWGPFDLVIGGSPCNDLSIVNPARKGLYEGTGRLFFEFYRLLHDARPKEG
DDRPFFWLFENVVAMGVSDKRDISRFLESNPVMIDAKEVSAAHRARYFWGNLPGMNRPLASTVNDKLE
LQECLEHGRIAKFSKVRTITTRSNSIKQGKDQHFPVFMNEKEDILWCTEMERVFGFPVHYTDVSNMSR
LARQRLLGRSWSVPVIRHLFAPLKEYFACVSSGNSNANSRGPSFSSGLVPLSLRGSHMGPMEIYKTVS
AWKRQPVRVLSLFRNIDKVLKSLGFLESGSGSGGGILKYVEDVINVVRRDVEKWGPFDLVYGSTQPLG
SSCDRCPGWYMFQFHRILQYALPRQESQRPFFWIEMDNLLLTEDDQETTTRFLQTEAVTLQDVRGRDY
QNAMRVWSNIPGLKSKHAPLTPKEEEYLQAQVRSRSKLDAPKVDLLVKNCLLPLREYFKYFSQNSLPL
GGPSSGAPPPSGGSPAGSPTSTEEGTSESATPESGPGTSTEPSEGSAPGSPAGSPTSTEEGTSTEPSE
GSAPGTSTEPSEPKKKRKVYMDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSLGLIPNF
KSNFDLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILIFRIPYYVGPLA
RGNSRFAWMTRKSEETITPWNFEEVVDKGASAQSFIERMTNFDKNLPNEKVLPKHSLLYEYFTVYNEL
TKVKYVTEGMRKPAFLSGEQKKAIVDLLFKTNRKVTVKQLKEDYFKKIECFDSVEISGVEDRFNASLG
TYHDLLKIIKDKDFLDNEENEDILEDIVLTLILFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIEEGIKELGSQIL
KEHPVENTQLQNEKLYLYYLQNGRDMYVDQELDINRLSDYDVDAIVPQSFLKDDSIDNKVLIRSDKNR
GKSDNVPSEEVVKKMKNYWRQLLNAKLITQRKFDNLTKAERGGLSELDKAGFIKRQLVETRQITKHVA
QILDSRMNTKYDENDKLIREVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEITLANGEIRKRPLIETNGE
TGEIVWDKGRDFATVRKVLSMPQVNIVKKTEVQTGGFSKESILPKRNSDKLIARKKDWDPKKYGGFDS
PTVAYSVLVVAKVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLINLGAPAAFKYFDTTIDRKRYTSTK
EVLDATLIHQSITGLYETRIDLSQLGGDPKKKRKVSGSETPGTSESATPESTGDSVAFEDVAVNFTLE
EWALLDPSQKNLYRDVMRETFRNLASVGKQWEDQNIEDPFKIPRRNISHIPERLCESKEGGQGEE.

1. An adjustable therapeutic gene therapy system for the delivery to, for example, any one of the tissues or cells provided herein, such as to the subcutaneous space (e.g., to one or more cell types in the subcutaneous space), which system comprises:
a. a genetic construct encoding at least one therapeutic gene, under the regulation of at least one promoter element and, optionally, one or more regulatory elements,
b. a delivery carrier or vector encapsulating at least part of said genetic construct, optionally, which allows for the re-administration of a subsequent dose of the genetic construct (e.g., at an efficiency of at least 10% as compared to an initial dose of the genetic construct), and
c. a means of increasing or decreasing the level of therapeutic transgene expression from the genetic construct.

2. The adjustable therapeutic gene therapy system of claim 1, wherein the means of c) is a means for permanently reducing the level of therapeutic transgene expression (e.g., by at least 2%).

3. The adjustable therapeutic gene therapy system of claim 1, wherein the means for reducing the level of therapeutic transgene expression is via application of an external stimulus.

4. The adjustable therapeutic gene therapy system of claim 1, wherein the genetic construct encodes at least one functional part of a human peptide or protein (e.g., a full-length human protein).

5-17. (canceled)

18. The adjustable therapeutic gene therapy system of claim 1, wherein the subcutaneous space is the hypodermal layer.

19-20. (canceled)

21. The adjustable therapeutic gene therapy system of claim 1, wherein the genetic construct is comprised of one or more circular single-stranded DNA constructs.

22-26. (canceled)

27. The adjustable therapeutic gene therapy system of claim 1, wherein the external stimulus is comprised of chemical DNA-, RNA-, or DNA- and RNA-derivatives.

28. The adjustable therapeutic gene therapy system of claim 1, wherein the promoter element comprises a constitutive promoter.

29-35. (canceled)

36. The adjustable therapeutic gene therapy system of claim 1, wherein the regulatory element comprises a post translational regulatory element.

37-42. (canceled)

43. The adjustable therapeutic gene therapy system of claim 1, wherein the genetic construct comprises one or more functional genetic sequences.

44-64. (canceled)

65. The adjustable therapeutic gene therapy system of claim 1, wherein the regulatory element changes the expression from the genetic construct in response to temperature.

66-77. (canceled)

78. The adjustable therapeutic gene therapy system of claim 1, wherein the delivery carrier or vector comprises lipid nanoparticles (e.g., functionalized lipid nanoparticles, solid lipid nanoparticles, lipid-polymer hybrid nanoparticles).

79-107. (canceled)

108. The adjustable therapeutic gene therapy system of claim 1, wherein the external stimulus elicits cooling of cutaneous and sub-cutaneous tissues (e.g., to cause adipocyte cell death).

109-147. (canceled)

148. A method of therapeutic transgene expression comprising, administering the adjustable therapeutic gene therapy system of claim 1 at least once.

149. A method of therapeutic transgene expression comprising, administering a genetic construct encoding at least one therapeutic gene, under the regulation of at least one promoter element and, optionally, one or more regulatory elements, and, optionally, a delivery carrier or vector encapsulating at least part of said genetic construct, to any one of the tissues or cells provided herein, such as to the subcutaneous space (e.g., to one or more cell types in the subcutaneous space).

150-152. (canceled)

153. The method of claim 148, wherein the method further comprises killing, reducing, removing or causing senescence of cells in the subcutaneous space to which the genetic construct has been administered.

154. The method of claim 148, wherein the method further comprises decreasing transgene expression from the genetic construct.

155. The method of claim 148, wherein the method further comprises increasing transgene expression from the genetic construct.

156-159. (canceled)

160. The method of claim 148, wherein the method further comprises administering a second dose of the i) adjustable gene therapy system or ii) the genetic construct encoding at least one therapeutic gene, under the regulation of at least one promoter element and, optionally, one or more regulatory elements, and, optionally, a delivery carrier or vector encapsulating at least part of said genetic construct, to the subcutaneous space (e.g., to one or more cell types in the subcutaneous space).

161-191. (canceled)

192. The adjustable therapeutic gene therapy system or method of claim 1, further comprising, or comprising the administration thereof, one or more markers for subsequent localization, visualization, analysis, enablement of up-titration, enablement of down-titration, selection of subsequent dosing, or general visualization, or the means for the foregoing.

193-196. (canceled)


SEQUENCE LISTING (78 sequences)

SEQ ID NO: 1 - DNA, 589 bp, synthetic construct
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 2 - DNA, 798 bp, synthetic construct
  source 1..798: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 3 - DNA, 232 bp, synthetic construct
  source 1..232: mol_type = other DNA
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 4 - DNA, 581 bp, synthetic construct
  source 1..581: mol_type = other DNA
gagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 5 - DNA, 598 bp, synthetic construct
  source 1..598: mol_type = other DNA
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 6 - DNA, 72 bp, synthetic construct
  source 1..72: mol_type = other DNA
ggtgtggaaagtccccaggctccccagcaggcagaagtatgcaaagcatgcatctcaattagtcagcaacca

SEQ ID NO: 7 - DNA, 46 bp, synthetic construct
  source 1..46: mol_type = other DNA
ctggggactttccagcctggggactttccagctgggactttccagg

SEQ ID NO: 8 - DNA, 47 bp, synthetic construct
  source 1..47: mol_type = other DNA
ctggggactttccagctggggactttccagctgggactttccaggag

SEQ ID NO: 9 - DNA, 134 bp, synthetic construct
  source 1..134: mol_type = other DNA
caaacaaacaaaggcgcgtcctggattcgtggtaaaacataccagatttcgatctggagaggtgaagaatacgaccacctactacatccagctgatgagtcccaaataggacgaaacgcgctcaaacaaacaaa

SEQ ID NO: 10 - protein, 1474 aa, synthetic construct
YMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVSGSETPGTSESATPESTGDSVAFEDVAVNFTLEEWALLDPSQKNLYRDVMRETFRNLASVGKQWEDQNIEDPFKIPRRNISHIPERLCESKEGGQGEE

SEQ ID NO: 11 - protein, 660 aa, synthetic construct [sequence omitted]

SEQ ID NO: 12 - protein, 830 aa, synthetic construct
MAPKKKRKVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSGGGGSGGGGSLEPGEKPYKCPECGKSFSDPGHLVRHQRTHTGEKPYKCPECGKSFSDCRDLARHQRTHTGEKPYKCPECGKSFSSPADLTRHQRTHTGEKPYKCPECGKSFSRADNLTEHQRTHTGEKPYKCPECGKSFSQSGHLTEHQRTHTGEKPYKCPECGKSFSRSDHLTTHQRTHTGKKTSSGGGGSGGGGSDSVAFEDVAVNFTLEEWALLDPSQKNLYRDVMRETFRNLASVGKQWEDQNIEDPFKIPRRNISHIPERLCESKEGGQGEE

SEQ ID NO: 13 - protein, 517 aa, synthetic construct
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLEYSGGGSLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 14 - DNA, 6483 bp, synthetic construct
  source 1..6483: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatcaactttgtatagaaaagttgctggggactttccagcctggggactttccagctgggactttccaggcaactttgtatagaaaagttgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgcaagtttgtacaaaaaagcaggctgccaccatgcacggcgagggcacattcaccagcgacctgagcaagcagatggaagaggaagccgtgagactgttcatcgagtggctgaagaacggcggaccctccagcggcgcccctccttctaagaaaaagaaaaagaagtaaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggccggcccgccgctttcgcactttagctgtttctccaggccacatatgattagttccaggccgaaaaggaaggcaggttcggctccctgccggtcgaacagctcaattgcttgtctcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggaatgacttggcgttgttccgcagaaagtcttgccatgactcgccttccagggggcagaagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcgccgtttatgactttgctcttgtccagtctagacatggggccgggattttcctccacgtccccgcatgttagaagacttcccctgccctctccggagccccagggatcctctccttgctgcaacagggagatcacctttggtttggtaaacaggaatcctgccatggaggccaggttgctgtaattctccagcatcacctcacggtacaggcttctctgagacagatccagcttcttccactcgtcccgagtaaagagcacagccacatcttcaaatgtcactgacactgctagctttctcttcttttttggtttattaccatcctcaatgggtgtatgctttttttcatctaactgtgcagaaaatccaaatattaatgatttcagcccgaaatggaaggccatttcagcagaggttctcgcctgaatattaaatgcctcttgtaataatggtggtgcagcgtctatcacatgattattttctatctggtttgttgcttgctcctctaatactgcacccaacgtaaaatggctgatagattgcagaatgaaaagagcctcctcgaccgaaaaccctgcatcgcatagacagcgtagttgcgcctctgcttgttcaaactgggggggcgtaggagaggtccctatatgcaatcgggctccatcacgatggaccagtaatgctttacggaaactcagagcattttcctggagaaactgctgccaactctcagtcggtaacggtgctgaacgggtgtgatgcttcgccagtattgcctctgaaagcatgttcataagagtctgcttgttgcgcacgtgccaatacaatgtaggctgctctacacctagcttctgggcgagtttacgggttgttaaaccttcgattccgacctcattaagcagctctaatgcgctgttaatcactttacttttatctaatctagacatgaattcccaacctgaaaaaaagtgatttcaggcaggtgctccaggtaattaaacattaataccccaccaaccaaccatcccttaaacccttacctcttgctcagctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcacaatgccaggcgggccatttaccgtcattgacgtcaatagggggcgtacttggcatatgatacacttgatgtactgccaagtgggcagtttaccgtaaatactccacccattgacgtcaatggaaagtccctattggcgttactattgacgtcaatgggcgggggtcgttgggcggtcagccaggcgggccatttaccgtaagttatgtaacggggccgcgggccgccgatcgaacttgtttattgcagcttataatggttacaaataaagcaatagcatcacaaatttcacaaataaagcatttttttcactgcattctagttgtggtttgtccaaactcatcaatgtatcttatttgtttgtttgagcgcgtttcgtcctatttgggactcatcagctggatgtagtaggtggtcgtattcttcacctctccagatcgaaatctggtatgttttaccacgaatccaggacgcgcctttgtttgtttggactagaagcggccgcctattagttggcctctcccatttcccgggcgaatgttctggccaggtcgcagatggtagggatagatccaggtgtggtcacgtgggtctggatcatgccgcttgtcagttgcagcagtgcatctctacatccggcaggagactggtcgtagtccaggatgaacacgtgcataggcctcagtcttttagccagcacgtcgagggcccaggcaaacacgttgtacaggtcgccgttaggggccagcagctcaggggctctgaacagggtaaacagcgtatcgccgatgtggggtctagggccggcgttgctttgaggctcggcgccctgggggggcacggcggtcccgctcagctggccccagtcctctctccagcttccgccgccctgcaggtatctcacggtattggccagcaggccatacactctgcggatagcagccagcatggccagatccagtctctcgcctggccgctgccgcttggccagtctatcgatgtgtctatcttctggcagggcgcccagcacgatgtttgtgccaggcagggtgggggggatcagggccacgaaggccagcacggcctgaggtgtcatggagcccatcaggtatctagcagcggggtagcacagcagagcggcgatggggtgccggtcgaagataagggtcagagctggaggaggggcgtggctgctaccggcttcgccgccaatatgaggagccagcacggcgtcggtgacggcgtagggcatgcccatggtgatctgggcgctggtcatcaccacggcggcgtcgccagcgctgatctcgccctgatccagtctgtgctgagtggtgtagatattggcgatggtttcagaggctcccaggacccgccagtaggtcatgggctcaggcacgtacacaatgtcgtctctggagcccagggccacgagcagctgtgttgtggtggtcttgcccatgccgtgagggccgtcgatgtaaacccgcagcagtgtaggcatggtggcagcctgcttttttgtacaaacttgtcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcccaacttttctatacaaagttgtctagactggggactttccagctggggactttccagctgggactttccaggagagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 16 - DNA, 424 bp, synthetic construct
  source 1..424: mol_type = other DNA
ggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattg

SEQ ID NO: 17 - DNA, 10 bp, synthetic construct
  source 1..10: mol_type = other DNA
gccaccatgg

SEQ ID NO: 18 - DNA, 138 bp, synthetic construct
  source 1..138: mol_type = other DNA
atgcacggcgagggcacattcaccagcgacctgagcaagcagatggaagaggaagccgtgagactgttcatcgagtggctgaagaacggcggaccctccagcggcgcccctccttctaagaaaaagaaaaagaagtaa

SEQ ID NO: 19 - protein, 45 aa, synthetic construct
MHGEGTFTSDLSKQMEEEAVRLFIEWLKNGGPSSGAPPSKKKKKK

SEQ ID NO: 20 - DNA, 222 bp, synthetic construct
  source 1..222: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 21 - DNA, 846 bp, synthetic construct
  source 1..846: mol_type = other DNA
atgtctagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaaccaaaaaagaagagaaagctagcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctgg

SEQ ID NO: 22 - protein, 282 aa, synthetic construct
MSRLDKSKVINSALELLNEVGIEGLTTRKLAQKLGVEQPTLYWHVRNKQTLMNMLSEAILAKHHTRSAPLPTESWQQFLQENALSFRKALLVHRDGARLHIGTSPTPPQFEQAEAQLRCLCDAGFSVEEALFILQSISHFTLGAVLEEQATNQIENNHVIDAAPPLLQEAFNIQARTSAEMAFHFGLKSLIFGFSAQLDEKKHTPIEDGNKPKKKRKLAVSVTFEDVAVLFTRDEWKKLDLSQRSLYREVMLENYSNLASMAGFLFTKPKVISLLQQGEDPW

SEQ ID NO: 23 - DNA, 54 bp, synthetic construct
  source 1..54: mol_type = other DNA
gagggcaggggaagtcttctaacatgcggggacgtggaggaaaatcccggcccc

SEQ ID NO: 24 - protein, 18 aa, synthetic construct
EGRGSLLTCGDVEENPGP

SEQ ID NO: 25 - DNA, 747 bp, synthetic construct
  source 1..747: mol_type = other DNA
atgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 26 - protein, 248 aa, synthetic construct
MSRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 27 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 28 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 29 - DNA, 996 bp, synthetic construct
  source 1..996: mol_type = other DNA
atgcctacactgctgcgggtttacatcgacggccctcacggcatgggcaagaccaccacaacacagctgctcgtggccctgggctccagagacgacattgtgtacgtgcctgagcccatgacctactggcgggtcctgggagcctctgaaaccatcgccaatatctacaccactcagcacagactggatcagggcgagatcagcgctggcgacgccgccgtggtgatgaccagcgcccagatcaccatgggcatgccctacgccgtcaccgacgccgtgctggctcctcatattggcggcgaagccggtagcagccacgcccctcctccagctctgacccttatcttcgaccggcaccccatcgccgctctgctgtgctaccccgctgctagatacctgatgggctccatgacacctcaggccgtgctggccttcgtggccctgatcccccccaccctgcctggcacaaacatcgtgctgggcgccctgccagaagatagacacatcgatagactggccaagcggcagcggccaggcgagagactggatctggccatgctggctgctatccgcagagtgtatggcctgctggccaataccgtgagatacctgcagggcggcggaagctggagagaggactggggccagctgagcgggaccgccgtgcccccccagggcgccgagcctcaaagcaacgccggccctagaccccacatcggcgatacgctgtttaccctgttcagagcccctgagctgctggcccctaacggcgacctgtacaacgtgtttgcctgggccctcgacgtgctggctaaaagactgaggcctatgcacgtgttcatcctggactacgaccagtctcctgccggatgtagagatgcactgctgcaactgacaagcggcatgatccagacccacgtgaccacacctggatctatccctaccatctgcgacctggccagaacattcgcccgggaaatgggagaggccaactaa

SEQ ID NO: 30 - protein, 331 aa, synthetic construct
MPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMIQTHVTTPGSIPTICDLARTFAREMGEAN

SEQ ID NO: 31 - DNA, 12 bp, synthetic construct
  source 1..12: mol_type = other DNA
caaacaaacaaa

SEQ ID NO: 32 - DNA, 110 bp, synthetic construct
  source 1..110: mol_type = other DNA
ggcgcgtcctggattcgtggtaaaacataccagatttcgatctggagaggtgaagaatacgaccacctactacatccagctgatgagtcccaaataggacgaaacgcgct

SEQ ID NO: 33 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 34 - DNA, 8199 bp, synthetic construct
  source 1..8199: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagaggtgtggaaagtccccaggctccccagcaggcagaagtatgcaaagcatgcatctcaattagtcagcaaccacaactttgtatagaaaagttgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgttggacgcgtgtcgacgccaccgccaccatggccctgtggatgagactgctgcctctgctggccctcctggccctgtggggccccgaccccgccgccgcttttgtgaaccagcacctgtgcggaagcgacctggtcgaggccctgtacctggtgtgcggcgagcggggcttcttctacacccctagaaccaagcgggaagccgaggatctgcaggtgggccaggtggagctgggcggcggaccaggcgccggctccctgcaacctctggctctggaaggatctagacagaagagaggcatcgtggagcagtgttgtacaagcatctgcagcctgtatcagctggaaaactactgcaattaaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgccgatcgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatctcgcctggagacgccatccacgctgttttgacctccatagaagacaccgggaccgatccagcctccgcggccgggaacggtgcattggaacgcggattccccgtgttaattaacaggtaagtgtcttcctcctgtttccttcccctgctattctgctcaaccttcctatcagaaactgcagtatctgtatttttgctagcagtaatactaacggttctttttttctcttcacaggccaccaagctaccggtccaccatggactcccagcagccagatctgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttagaattcccgggtgtcgaccagaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgggtcgacgcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaaggatcctccggactagaaaagccgaattctggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttcaagtttgtacaaaaaagcaggctaacttgtttattgcagcttataatggttacaaataaagcaatagcatcacaaatttcacaaataaagcatttttttcactgcattctagttgtggtttgtccaaactcatcaatgtatcttatttgtttgtttgagcgcgtttcgtcctatttgggactcatcagctggatgtagtaggtggtcgtattcttcacctctccagatcgaaatctggtatgttttaccacgaatccaggacgcgcctttgtttgtttggactagaagcggccgcctattagttggcctctcccatttcccgggcgaatgttctggccaggtcgcagatggtagggatagatccaggtgtggtcacgtgggtctggatcatgccgcttgtcagttgcagcagtgcatctctacatccggcaggagactggtcgtagtccaggatgaacacgtgcataggcctcagtcttttagccagcacgtcgagggcccaggcaaacacgttgtacaggtcgccgttaggggccagcagctcaggggctctgaacagggtaaacagcgtatcgccgatgtggggtctagggccggcgttgctttgaggctcggcgccctgggggggcacggcggtcccgctcagctggccccagtcctctctccagcttccgccgccctgcaggtatctcacggtattggccagcaggccatacactctgcggatagcagccagcatggccagatccagtctctcgcctggccgctgccgcttggccagtctatcgatgtgtctatcttctggcagggcgcccagcacgatgtttgtgccaggcagggtgggggggatcagggccacgaaggccagcacggcctgaggtgtcatggagcccatcaggtatctagcagcggggtagcacagcagagcggcgatggggtgccggtcgaagataagggtcagagctggaggaggggcgtggctgctaccggcttcgccgccaatatgaggagccagcacggcgtcggtgacggcgtagggcatgcccatggtgatctgggcgctggtcatcaccacggcggcgtcgccagcgctgatctcgccctgatccagtctgtgctgagtggtgtagatattggcgatggtttcagaggctcccaggacccgccagtaggtcatgggctcaggcacgtacacaatgtcgtctctggagcccagggccacgagcagctgtgttgtggtggtcttgcccatgccgtgagggccgtcgatgtaaacccgcagcagtgtaggcatggtggcagcctgcttttttgtacaaacttgagagtgaggacgaacgcccccacccccttttatagccccccgctagcgtcttcggaggacagtactccggaattcgcggaggacagtactccgatcggaggacagtactccgctcgagtcggaggacagtactccgctcggaggacagtactccgcaacttttctatacaaagttgtctagaggtgtggaaagtccccaggctccccagcaggcagaagtatgcaaagcatgcatctcaattagtcagcaaccaggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 35 - DNA, 1713 bp, synthetic construct
  source 1..1713: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgc

SEQ ID NO: 36 - DNA, 333 bp, synthetic construct
  source 1..333: mol_type = other DNA
atggccctgtggatgagactgctgcctctgctggccctcctggccctgtggggccccgaccccgccgccgcttttgtgaaccagcacctgtgcggaagcgacctggtcgaggccctgtacctggtgtgcggcgagcggggcttcttctacacccctagaaccaagcgggaagccgaggatctgcaggtgggccaggtggagctgggcggcggaccaggcgccggctccctgcaacctctggctctggaaggatctagacagaagagaggcatcgtggagcagtgttgtacaagcatctgcagcctgtatcagctggaaaactactgcaattaa

SEQ ID NO: 37 - protein, 110 aa, synthetic construct
MALWMRLLPLLALLALWGPDPAAAFVNQHLCGSDLVEALYLVCGERGFFYTPRTKREAEDLQVGQVELGGGPGAGSLQPLALEGSRQKRGIVEQCCTSICSLYQLENYCN

SEQ ID NO: 38 - DNA, 118 bp, synthetic construct
  source 1..118: mol_type = other DNA
gtaagtgtcttcctcctgtttccttcccctgctattctgctcaaccttcctatcagaaactgcagtatctgtatttttgctagcagtaatactaacggttctttttttctcttcacag

SEQ ID NO: 39 - DNA, 1965 bp, synthetic construct
  source 1..1965: mol_type = other DNA
atggactcccagcagccagatctgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttagaattcccgggtgtcgaccagaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgggtcgacgcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaa

SEQ ID NO: 40 - protein, 654 aa, synthetic construct
MDSQQPDLKLLSSIEQACDICRLKKLKCSKEKPKCAKCLKNNWECRYSPKTKRSPLTRAHLTEVESRLERLEQLFLLIFPREDLDMILKMDSLQDIKALLEFPGVDQKKFNKVRVVRALDAVALPQPVGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAGSTPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 41 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 42 - DNA, 103 bp, synthetic construct
  source 1..103: mol_type = other DNA
cggagtactgtcctccgagcggagtactgtcctccgactcgagcggagtactgtcctccgatcggagtactgtcctccgcgaattccggagtactgtcctccg

SEQ ID NO: 43 - DNA, 41 bp, synthetic construct
  source 1..41: mol_type = other DNA
ggggggctataaaagggggtgggggcgttcgtcctcactct

SEQ ID NO: 44 - DNA, 4554 bp, synthetic construct
  source 1..4554: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagatggggactttccgctggggactttccgctggggactttccgctggggactttccgctggggactttccgccaactttgtatagaaaagttggggaatttccggggactttccgggaatttccggggactttccgggaatttccagatctggcctcggcggccaagcttagacactagagggtatataatggaagctcgacttccagcaagtttgtacaaaaaagcaggctgtaagggtttaagggatggttggttggtggggtattaatgtttaattacctggagcacctgcctgaaatcactttttttcaggttggacgcgtgtcgacgccaccgccaccatggaaatctgtagaggcctgaggtctcatctgattaccctgctgctgttcctgttccacagcgaaaccatctgcagacctagcggcagaaagagcagcaagatgcaagctttcagaatctgggatgtgaaccagaagaccttctacctgcggaacaaccagctggtggccggctacctgcagggccctaatgtgaatctggaagagaaaatcgacgtcgtgcccatcgagcctcacgccctgtttctgggcatccacggcggcaagatgtgcctgagctgcgtgaagtctggagatgagacaagactgcagctcgaggccgtgaacatcacagacctgtccgagaacagaaaacaggacaagcggttcgccttcatccggagcgacagcggaccaaccaccagcttcgagtctgccgcttgtcccggctggttcctgtgcacagccatggaagccgaccagcctgtgtccctgaccaacatgcctgatgaaggcgtgatggtgaccaagttctactttcaggaggacgagtaaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgccgatcgaacttgtttattgcagcttataatggttacaaataaagcaatagcatcacaaatttcacaaataaagcatttttttcactgcattctagttgtggtttgtccaaactcatcaatgtatcttatttgtttgtttgagcgcgtttcgtcctatttgggactcatcagctggatgtagtaggtggtcgtattcttcacctctccagatcgaaatctggtatgttttaccacgaatccaggacgcgcctttgtttgtttggactagaagcggccgcctattagctggcgctggtcttgaaaaacagcttctttctcaagaagttgaagcagccaggcatctgcttgtagatgcccttaacggacacggcattagccacccgcagcagcagagattgaaggtcctcgctgtgggcccactgctcaaagatatcgtccagtgtctccacgtaccaggagccgctcttagggtcccgccagctcacgaatcctggaaaggtgctatatgacacgaagatatcgctaggtgtaggcaggctggagatggcatccagctggtcaaaagttctcaggccctcctgaaaaggggtggcgtctggctcaggattagagcctggagactcgtcctcggggcttgtgctggccacctcgaagccgtggtccttctgttcgccgccacaagcctggatgaagaacagcttgggcttgccgcccaggctggggcaggatgtgccgttgaagatgttcacgatcttttccacggacacagggcaaccgtcggtgccgtacactgctccagggaactgcaggtggctagcctgacagccatggctcaggatgaccaccacgcagcagtccagggcgccgtggtcctgctgggccagttcgagcagggccagcaccatcttcttggcggtgagatctcctttcacctcgaccatgaagtgcagagagctgaatcttctccgcagcttctcgcagtcgatgttgctgccggttctggtgcgcagtccgctctctctgcagaagttcacgttgttgatgatcaggcagtggccacaaggctccatgctcagaatgtaagccagatcggcgttgcctctgaggctttccagggcgcccacgtcgccgaagccgtccacgccggaaccgccgccgccgctgccaccgcctccagagccgccgcctccgctctccagcttcagcagctccacatcaaagaccagggtggcgtggggggggatgatgccagggtgtccggtagcgccgtaggcgtagtcaggagaaattgtcagtttggcccgctggcccacgctcatttgggccacgccttcctcccagcctctgatcacctcctgtttgcccagcatgaacttgaaaggcttgttccggtctctgctgctgtcgaatttctttccatcttccagcattccggtgtagtgcaccacacaggtctgtcctcttttggggaatgttctgccatcgccaggagagatggtttccacctgaactccttccaggcttcctcctccgctgtattccagcttgctgatccgtctgaacacgtggtagtacaggtcccaggcttgcagcaggtccttcacattcccgcttttcatgtacttccggcaccactcctgggcttccatcagatcgcggccataggcctggttgaagcttgtttccttcagggtctgagggcctctttccatcatggcgtgcaggggttccaggacctcgaacatgcccttcacgttccgctcgccgaagtacagtctactagcctcttccaggccctcgtgccacatctcatgccacaggattctagaggccatggtggcagcctgcttttttgtacaaacttggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactacaacttttctatacaaagttgtctagatggggactttccgctggggactttccgctggggactttccgctggggactttccgctggggactttccgcggtaccaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 45 - DNA, 70 bp, synthetic construct
  source 1..70: mol_type = other DNA
tggggactttccgctggggactttccgctggggactttccgctggggactttccgctggggactttccgc

SEQ ID NO: 46 - DNA, 52 bp, synthetic construct
  source 1..52: mol_type = other DNA
gggaatttccggggactttccgggaatttccggggactttccgggaatttcc

SEQ ID NO: 47 - DNA, 32 bp, synthetic construct
  source 1..32: mol_type = other DNA
tagagggtatataatggaagctcgacttccag

SEQ ID NO: 48 - DNA, 82 bp, synthetic construct
  source 1..82: mol_type = other DNA
gtaagggtttaagggatggttggttggtggggtattaatgtttaattacctggagcacctgcctgaaatcactttttttcag

SEQ ID NO: 49 - DNA, 534 bp, synthetic construct
  source 1..534: mol_type = other DNA
atggaaatctgtagaggcctgaggtctcatctgattaccctgctgctgttcctgttccacagcgaaaccatctgcagacctagcggcagaaagagcagcaagatgcaagctttcagaatctgggatgtgaaccagaagaccttctacctgcggaacaaccagctggtggccggctacctgcagggccctaatgtgaatctggaagagaaaatcgacgtcgtgcccatcgagcctcacgccctgtttctgggcatccacggcggcaagatgtgcctgagctgcgtgaagtctggagatgagacaagactgcagctcgaggccgtgaacatcacagacctgtccgagaacagaaaacaggacaagcggttcgccttcatccggagcgacagcggaccaaccaccagcttcgagtctgccgcttgtcccggctggttcctgtgcacagccatggaagccgaccagcctgtgtccctgaccaacatgcctgatgaaggcgtgatggtgaccaagttctactttcaggaggacgagtaa

SEQ ID NO: 50 - protein, 177 aa, synthetic construct
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 51 - DNA, 1554 bp, synthetic construct
  source 1..1554: mol_type = other DNA
atggcctctagaatcctgtggcatgagatgtggcacgagggcctggaagaggctagtagactgtacttcggcgagcggaacgtgaagggcatgttcgaggtcctggaacccctgcacgccatgatggaaagaggccctcagaccctgaaggaaacaagcttcaaccaggcctatggccgcgatctgatggaagcccaggagtggtgccggaagtacatgaaaagcgggaatgtgaaggacctgctgcaagcctgggacctgtactaccacgtgttcagacggatcagcaagctggaatacagcggaggaggaagcctggaaggagttcaggtggaaaccatctctcctggcgatggcagaacattccccaaaagaggacagacctgtgtggtgcactacaccggaatgctggaagatggaaagaaattcgacagcagcagagaccggaacaagcctttcaagttcatgctgggcaaacaggaggtgatcagaggctgggaggaaggcgtggcccaaatgagcgtgggccagcgggccaaactgacaatttctcctgactacgcctacggcgctaccggacaccctggcatcatccccccccacgccaccctggtctttgatgtggagctgctgaagctggagagcggaggcggcggctctggaggcggtggcagcggcggcggcggttccggcgtggacggcttcggcgacgtgggcgccctggaaagcctcagaggcaacgccgatctggcttacattctgagcatggagccttgtggccactgcctgatcatcaacaacgtgaacttctgcagagagagcggactgcgcaccagaaccggcagcaacatcgactgcgagaagctgcggagaagattcagctctctgcacttcatggtcgaggtgaaaggagatctcaccgccaagaagatggtgctggccctgctcgaactggcccagcaggaccacggcgccctggactgctgcgtggtggtcatcctgagccatggctgtcaggctagccacctgcagttccctggagcagtgtacggcaccgacggttgccctgtgtccgtggaaaagatcgtgaacatcttcaacggcacatcctgccccagcctgggcggcaagcccaagctgttcttcatccaggcttgtggcggcgaacagaaggaccacggcttcgaggtggccagcacaagccccgaggacgagtctccaggctctaatcctgagccagacgccaccccttttcaggagggcctgagaacttttgaccagctggatgccatctccagcctgcctacacctagcgatatcttcgtgtcatatagcacctttccaggattcgtgagctggcgggaccctaagagcggctcctggtacgtggagacactggacgatatctttgagcagtgggcccacagcgaggaccttcaatctctgctgctgcgggtggctaatgccgtgtccgttaagggcatctacaagcagatgcctggctgcttcaacttcttgagaaagaagctgtttttcaagaccagcgccagctaa

SEQ ID NO: 52 - DNA, 7975 bp, synthetic construct
  source 1..7975: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatcaactttgtatagaaaagttgggtacattttgttctagaacaaaatgtaccggtacattttgttctggtacattttgttctcaactttgtatagaaaagttgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattgcaagtttgtacaaaaaagcaggctgccaccatggcctctagaatcctgtggcatgagatgtggcacgagggcctggaagaggctagtagactgtacttcggcgagcggaacgtgaagggcatgttcgaggtcctggaacccctgcacgccatgatggaaagaggccctcagaccctgaaggaaacaagcttcaaccaggcctatggccgcgatctgatggaagcccaggagtggtgccggaagtacatgaaaagcgggaatgtgaaggacctgctgcaagcctgggacctgtactaccacgtgttcagacggatcagcaagctggaatacagcggaggaggaagcctggaaggagttcaggtggaaaccatctctcctggcgatggcagaacattccccaaaagaggacagacctgtgtggtgcactacaccggcatgctggaagatggaaagaaattcgacagcagcagagaccggaacaagcctttcaagttcatgctgggcaaacaggaggtgatcagaggctgggaggaaggcgtggcccaaatgagcgtgggccagcgggccaaactgacaatttctcctgactacgcctacggcgctaccggacaccctggcatcatccccccccacgccaccctggtctttgatgtggagctgctgaagctggagagcggaggcggcggctctggaggcggtggcagcggcggcggcggttccggcgtggacggcttcggcgacgtgggcgccctggaaagcctcagaggcaacgccgatctggcttacattctgagcatggagccttgtggccactgcctgatcatcaacaacgtgaacttctgcagagagagcggactgcgcaccagaaccggcagcaacatcgactgcgagaagctgcggagaagattcagctctctgcacttcatggtcgaggtgaaaggagatctcaccgccaagaagatggtgctggccctgctcgaactggcccagcaggaccacggcgccctggactgctgcgtggtggtcatcctgagccatggctgtcaggctagccacctgcagttccctggagcagtgtacggcaccgacggttgccctgtgtccgtggaaaagatcgtgaacatcttcaacggcacatcctgccccagcctgggcggcaagcccaagctgttcttcatccaggcttgtggcggcgaacagaaggaccacggcttcgaggtggccagcacaagccccgaggacgagtctccaggctctaatcctgagccagacgccaccccttttcaggagggcctgagaacttttgaccagctggatgccatctccagcctgcctacacctagcgatatcttcgtgtcatatagcacctttccaggattcgtgagctggcgggaccctaagagcggctcctggtacgtggagacactggacgatatctttgagcagtgggcccacagcgaggaccttcaatctctgctgctgcgggtggctaatgccgtgtccgttaagggcatctacaagcagatgcctggctgcttcaacttcttgagaaagaagctgtttttcaagaccagcgccagctaaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggccggcccgccgctttcgcactttagctgtttctccaggccacatatgattagttccaggccgaaaaggaaggcaggttcggctccctgccggtcgaacagctcaattgcttgtctcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggaatgacttggcgttgttccgcagaaagtcttgccatgactcgccttccagggggcagaagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcgccgtttatgactttgctcttgtccagtctagacatggggccgggattttcctccacgtccccgcatgttagaagacttcccctgccctctccggagccccagggatcctctccttgctgcaacagggagatcacctttggtttggtaaacaggaatcctgccatggaggccaggttgctgtaattctccagcatcacctcacggtacaggcttctctgagacagatccagcttcttccactcgtcccgagtaaagagcacagccacatcttcaaatgtcactgacactgctagctttctcttcttttttggtttattaccatcctcaatgggtgtatgctttttttcatctaactgtgcagaaaatccaaatattaatgatttcagcccgaaatggaaggccatttcagcagaggttctcgcctgaatattaaatgcctcttgtaataatggtggtgcagcgtctatcacatgattattttctatctggtttgttgcttgctcctctaatactgcacccaacgtaaaatggctgatagattgcagaatgaaaagagcctcctcgaccgaaaaccctgcatcgcatagacagcgtagttgcgcctctgcttgttcaaactgggggggcgtaggagaggtccctatatgcaatcgggctccatcacgatggaccagtaatgctttacggaaactcagagcattttcctggagaaactgctgccaactctcagtcggtaacggtgctgaacgggtgtgatgcttcgccagtattgcctctgaaagcatgttcataagagtctgcttgttgcgcacgtgccaatacaatgtaggctgctctacacctagcttctgggcgagtttacgggttgttaaaccttcgattccgacctcattaagcagctctaatgcgctgttaatcactttacttttatctaatctagacatgaattcccaacctgaaaaaaagtgatttcaggcaggtgctccaggtaattaaacattaataccccaccaaccaaccatcccttaaacccttacctcttgctcagctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcacaatgccaggcgggccatttaccgtcattgacgtcaatagggggcgtacttggcatatgatacacttgatgtactgccaagtgggcagtttaccgtaaatactccacccattgacgtcaatggaaagtccctattggcgttactattgacgtcaatgggcgggggtcgttgggcggtcagccaggcgggccatttaccgtaagttatgtaacggggccgcgggccgccgatcgaacttgtttattgcagcttataatggttacaaataaagcaatagcatcacaaatttcacaaataaagcatttttttcactgcattctagttgtggtttgtccaaactcatcaatgtatcttatttgtttgtttgagcgcgtttcgtcctatttgggactcatcagctggatgtagtaggtggtcgtattcttcacctctccagatcgaaatctggtatgttttaccacgaatccaggacgcgcctttgtttgtttggactagaagcggccgcctaccgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccatggaaaggacgtcagcttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcggaattcccactttgtacaagaaagctgggtctattatcttcctctcttaatggtcagggtacacacgcagctggtgtcgattctgatgaaccgccagccgatccgcttcttgctgtccattgtcagggcccgcacataagattgggtggttctgcactggctgttccagtggcgcttgtcgatgcctctacagccttcctttgtgtagcccatggggttgcattttgtctcgtagaagtactgcttcagctggcccttggacacaggcaccttttccagcacggtcactgttccgccgctcatgtccacggcggttttcttatcagcggctgtgacccactcgctgatgctgtcgcacacgctcagctcgccccttctggcaggatcagagtgggctttcatgcagccaaagtaggagatcaccatggtcaggaacaggatggtcatggtggcagcctgcttttttgtacaaacttgtcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcccaacttttctatacaaagttgtctagaggtacattttgttctagaacaaaatgtaccggtacattttgttctggtacattttgttctagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 53 - DNA, 417 bp, synthetic construct
  source 1..417: mol_type = other DNA
atgaccatcctgttcctgaccatggtgatctcctactttggctgcatgaaagcccactctgatcctgccagaaggggcgagctgagcgtgtgcgacagcatcagcgagtgggtcacagccgctgataagaaaaccgccgtggacatgagcggcggaacagtgaccgtgctggaaaaggtgcctgtgtccaagggccagctgaagcagtacttctacgagacaaaatgcaaccccatgggctacacaaaggaaggctgtagaggcatcgacaagcgccactggaacagccagtgcagaaccacccaatcttatgtgcgggccctgacaatggacagcaagaagcggatcggctggcggttcatcagaatcgacaccagctgcgtgtgtaccctgaccattaagagaggaagataatag

SEQ ID NO: 54 - protein, 137 aa, synthetic construct
MTILFLTMVISYFGCMKAHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 55 - DNA, 60 bp, synthetic construct
  source 1..60: mol_type = other DNA
ggtacattttgttctagaacaaaatgtaccggtacattttgttctggtacattttgttct

SEQ ID NO: 56 - DNA, 3677 bp, synthetic construct
  source 1..3677: mol_type = other DNA
ctggggactttccagcctggggactttccagctgggactttccaggggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggataacttcgtatagcatacattatacgaagttatgttggacgcgtcgctagcgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggagggccgccgatcgaacttgtttattgcagcttataatggttacaaataaagcaatagcatcacaaatttcacaaataaagcatttttttcactgcattctagttgtggtttgtccaaactcatcaatgtatcttatttgtttgtttgagcgcgtttcgtcctatttgggactcatcagctggatgtagtaggtggtcgtattcttcacctctccagatcgaaatctggtatgttttaccacgaatccaggacgcgcctttgtttgtttggactagaagcggccgcctatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgcctgatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggcagcctgcttttttgtacaaacttgataacttcgtatagcatacattatacgaagttatcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggcgatgtgcgctctgcccactgacgggcaccggagccctggggactttccagctggggactttccagctgggactttccaggag

SEQ ID NO: 57 - DNA, 34 bp, synthetic construct
  source 1..34: mol_type = other DNA
ataacttcgtatagcatacattatacgaagttat

SEQ ID NO: 58 - DNA, 516 bp, synthetic construct
  source 1..516: mol_type = other DNA
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 59 - protein, 171 aa, synthetic construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 60 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctgggga

SEQ ID NO: 61 - DNA, 1983 bp, synthetic construct
  source 1..1983: mol_type = other DNA
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatctcgagccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttga

SEQ ID NO: 62 - DNA, 2103 bp, synthetic construct
  source 1..2103: mol_type = other DNA
ctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtaccctggggactttccagcctggggactttccagctgggactttccaggcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggttggacgcgtcgctagcgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcaggctggggactggggactttccagctggggactttccagctgggactttccaggaga

SEQ ID NO: 63 - protein, 301 aa, Homo sapiens
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 64 - protein, 214 aa, Mus musculus
GPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 65 - protein, 80 aa, synthetic construct
DSVAFEDVAVNFTLEEWALLDPSQKNLYRDVMRETFRNLASVGKQWEDQNIEDPFKIPRRNISHIPERLCESKEGGQGEE

SEQ ID NO: 66 - protein, 9 aa, synthetic construct
MAPKKKRKV

SEQ ID NO: 67 - protein, 11 aa, synthetic construct
SGGGGSGGGGS

SEQ ID NO: 68 - DNA, 18 bp, synthetic construct
  source 1..18: mol_type = other DNA
tggcgacagacagccggc

SEQ ID NO: 69 - DNA, 400 bp, synthetic construct
  source 1..400: mol_type = other DNA
ccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggttggacgcgtcgctagcgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtct

SEQ ID NO: 70 - protein, 176 aa, synthetic construct
LEPGEKPYKCPECGKSFSDPGHLVRHQRTHTGEKPYKCPECGKSFSDCRDLARHQRTHTGEKPYKCPECGKSFSSPADLTRHQRTHTGEKPYKCPECGKSFSRADNLTEHQRTHTGEKPYKCPECGKSFSQSGHLTEHQRTHTGEKPYKCPECGKSFSRSDHLTTHQRTHTGKKTS

SEQ ID NO: 71 - DNA, 2493 bp, synthetic construct
  source 1..2493: mol_type = other DNA
atggcccctaaaaaaaagagaaaggtcaaccacgaccaggagttcgacccacctaaggtgtaccctcctgtgccagccgagaagcggaagccaatccgggtgctgagcctctttgacggcatcgccaccggcctgctggtgctgaaggacctgggcatccaggtggacagatacatcgccagtgaagtgtgcgaagattctatcaccgtgggcatggtgcggcaccaaggcaagatcatgtacgtgggcgacgtccggtctgtgacccagaagcatatccaggagtggggccccttcgacctggttatcggcggaagcccctgcaacgacctgagcatcgtgaaccccgctagaaagggcctgtacgagggcacaggcagactgttcttcgagttctaccggctgctgcacgatgcccgtcccaaggaaggcgatgacagaccttttttctggctgttcgagaacgtggtggccatgggcgtgagcgacaaacgggacatcagcagattcctggagtctaaccctgtgatgattgatgctaaggaagtgtctgccgcccaccgggcccggtacttctggggcaacctgccaggcatgaacagacctctcgcctctaccgtgaatgacaagctggaactgcaggagtgtctggaacacggcagaatcgccaagttcagtaaggtgagaaccatcaccaccaggagcaacagcatcaagcagggcaaagatcagcacttccctgtgtttatgaacgagaaggaggacatcctgtggtgcaccgagatggaaagagtttttggcttccccgtgcactacaccgacgtttctaacatgagcagactggccagacagcggctgctgggcagatcctggagcgtgcctgtgatcagacacctgttcgcccccctgaaggagtactttgcctgcgtgagctccggaaattctaacgccaactcccgcggcccctccttcagcagcggactggtgcctctcagcctgagaggcagccacatgggacctatggaaatatacaagacagtgtcggcttggaagaggcagcctgtgagagtgctgagcctgttcagaaacatcgacaaggtgctgaagagcctgggatttctggaatctggaagcgggtctggcggcggcaccctgaagtacgtggaagatgtgacaaacgtggtgcggagagatgtcgagaaatggggacctttcgatctggtgtacggcagcacccagcccctgggcagcagctgcgaccggtgccccggctggtacatgtttcaattccaccgtatcctgcagtacgccctgcctcggcaggaaagccagagacctttcttctggatcttcatggacaacctgctgctgaccgaggacgaccaggagacaaccacaagattcctgcagaccgaggccgtgaccctgcaggatgtccggggccgcgactaccagaatgccatgcgggtgtggagcaatatccctggcctgaagtccaagcacgcccctctgacacctaaggaagaagagtacctgcaagctcaggtgagaagcagatccaagctggatgcccctaaggtcgacctgctggtgaaaaactgcctgctgccacttagagagtacttcaaatacttctcccaaaatagcctgcccctgagcggcggaggcgggagcggcggaggcggcagcctggagcctggcgagaagccctacaagtgcccagagtgcggcaaatcttttagcgaccctggccacctggtgcggcaccagcggacccataccggtgagaaaccctacaaatgtcctgaatgtggcaagagcttctccgattgcagagatctggcccggcaccagagaacccatacaggcgaaaaaccttacaagtgccctgagtgcggaaaatccttcagctctcctgccgacctgacaagacaccagcggacccacacaggcgagaaaccttataagtgtcccgaatgcggcaagagcttctccagagccgataacctgaccgagcaccaaaggacccacaccggcgaaaagccttataagtgcccagaatgcggcaagagcttttcccagagcggtcatctgacagagcaccagagaacacacaccggagagaagccctataagtgtcctgagtgtggaaaaagcttttcaagaagcgaccacctcaccacacaccagcgcacacacacaggcaagaagaccagcagcggtggcggcggctctggcggcggaggaagcgacagcgtggccttcgaggacgtggctgtgaatttcactctggaagagtgggctctgctcgaccctagccaaaagaacctgtacagagacgtgatgagagaaaccttcagaaacctggctagcgtcggcaaacagtgggaggatcagaacatcgaggatcctttcaagatcccgagacggaacatcagccacatcccagagagactgtgcgagtccaaggagggaggccagggcgaggaataa

SEQ ID NO: 72 - protein, 850 aa, synthetic construct
MAPKKKRKVMNHDOEFDPPKVYPPVPAEKRKPIRVLSLFDGIATGLLVLKDLGIOVDRYIASEVCEDSITVGMVRHOGKIMYVGDVRSVTOKHIOEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELOECLEHGRIAKFSKVRTITTRSNSIKOGKDOHFPVFMNEKEDILWCTEMERVFGFPVHYTDVSNMSRLARORLLGRSWSVPVIRHLFAPLKEYFACVSSGNSNANSRGPSFSSGLVPLSLRGSHMGPMEIYKTVSAWKROPVRVLSLFRNIDKVLKSLGFLESGSGSGGGTLKYVEDVTNVVRRDVEKWGPFDLVYGSTOPLGSSCDRCPGWYMFQFHRILOYALPRQESQRPFFWIFMDNLLLTEDDQETTTRFLOTEAVTLODVRGRDYNAMRVWSNIPGLKSKHAPLTPKEEEYLQAQVRSRSKLDAPKVDLLVKNCLLPLREYFKYFSQNSLPLSGGGGSGGGGSVGIHGVPSRPGERPFQCRICMRNFSHKSSLTRHTRTHTGEKPFQCRICMRNFSRTEHLARHLRTHTGSQKPFQCRICMRNFSQSAHLKRHTRTHTGEKPFQCRICMRNFSRTEHLARHLRTHTGGGGSQKPFQCRICMRNFSHKSSLTRHTRTHTGEKPFQCRICMRNFSRPESLAPHLRTHLRGSGGGSMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 73 - protein, 302 aa, synthetic construct
MNHDOEFDPPKVYPPVPAEKRKPIRVLSLFDGIATGLLVLKDLGIOVDRYIASEVCEDSITVGMVRHOGKIMYVGDVRSVTOKHIOEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELOECLEHGRIAKFSKVRTITTRSNSIKOGKDOHFPVFMNEKEDILWCTEMERVFGFPVHYTDVSNMSRLARORLLGRSWSVPVIRHLFAPLKEYFACV

SEQ ID NO: 74 - protein, 259 aa, synthetic construct
SSGNSNANSRGPSFSSGLVPLSLRGSHMGPMEIYKTVSAWKROPVRVLSLFRNIDKVLKSLGFLESGSGSGGGTLKYVEDVTNVVRRDVEKWGPFDLVYGSTOPLGSSCDRCPGWYMFQFHRILOYALPRQESQRPFFWIFMDNLLLTEDDQETTTRFLOTEAVTLODVRGRDYNAMRVWSNIPGLKSKHAPLTPKEEEYLQAQVRSRSKLDAPKVDLLVKNCLLPLREYFKYFSQNSLPLSGGGGSGGGGSVGIHGVP

SEQ ID NO: 75 - protein, 4 aa, synthetic construct
GGGS

SEQ ID NO: 76 - protein, 7 aa, synthetic construct
PKKKRKV

SEQ ID NO: 77 - protein, 179 aa, synthetic construct
SRPGERPFQCRICMRNFSHKSSLTRHTRTHTGEKPFQCRICMRNFSRTEHLARHLRTHTGSQKPFQCRICMRNFSQSAHLKRHTRTHTGEKPFQCRICMRNFSRTEHLARHLRTHTGGGGSQKPFQCRICMRNFSHKSSLTRHTRTHTGEKPFQCRICMRNFSRPESLAPHLRTHLRGS

SEQ ID NO: 78 - protein, 2105 aa, synthetic construct
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKROPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVYMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVSGSETPGTSESATPESTGDSVAFEDVAVNFTLEEWALLDPSQKNLYRDVMRETFRNLASVGKQWEDQNIEDPFKIPRRNISHIPERLCESKEGGQGEE